(19) United States
(12) Patent Application Publication
(10) Pub. No.: US 2015/0196002 A1
(43) Pub. Date: Jul. 16, 2015

(54) AUTOMATED HYBRID AQUAPONICS AND BIOREACTOR SYSTEM INCLUDING PRODUCT PROCESSING AND STORAGE FACILITIES WITH INTEGRATED ROBOTICS, CONTROL SYSTEM, AND RENEWABLE ENERGY SYSTEM CROSS-REFERENCE TO RELATED APPLICATIONS

(71) Applicant: Kevin Friesth, Fort Dodge, IA (US)

(72) Inventor: Kevin Friesth, Fort Dodge, IA (US)

(21) Appl. No.: 14/267,580

(22) Filed: May 1, 2014

Related U.S. Application Data
(60) Provisional application No. 61/926,372, filed on Jan. 12, 2014.

(51) Int. Cl. A01G 31/02; H05B 37/02; A01G 7/04

(52) U.S. Cl. CPC A01G 31/02; A01G 7/045; H05B 37/0245

(57) ABSTRACT

Provided is a consumer to industrial scale automated high-yield aquaponics system that amalgamates conventional farming techniques with hybrid integrated multi-trophic aquaculture with aeroponics. The present invention also includes a microalgae bioreactor and organism reactor production system. Further, the present invention utilizes adaptive metrics, biometrics, and thermal imaging analysis, monitoring, and control (using robotic automation) via an artificially intelligent control system. The control system of the present invention allows for a controlled, symbiotic environmental ecosystem. Additionally, the present invention incorporates integrated product processing, packaging, dry storage and cold storage facility and enhanced biosecurity. The present invention utilizes renewable green energy sources as the primary energy component input. Thus, the system of the present invention yields an environmental friendly, sealable, and sustainable aquaponics system with organically-derived and contaminate-free produce including, but not limited to fruits, vegetables, herbs, and flowers as well as a wide variety of microalgae, organisms, and aquaculture species.

### CROSS-REFERENCE TO RELATED APPLICATIONS

This application claims priority from U.S. Provisional Application Ser. No. 61/926,372 filed Jan. 12, 2014 and entitled AUTOMATED HYBRID AQUAPONICS AND BIOREACTOR SYSTEM INCLUDING PRODUCT PROCESSING AND STORAGE FACILITIES WITH INTEGRATED ROBOTICS, CONTROL SYSTEM, AND RENEWABLE ENERGY SYSTEM. The contents of U.S. Provisional Application Ser. No. 61/926,392 is hereby incorporated in its entirety by reference.

### FIELD OF THE INVENTION

The present invention relates to an automated hybrid aquaponics system encompassing an artificially intelligent controlled and stabilized environmental control system using adaptive biometrics and thermal imaging for active analysis, monitoring, and machine learning control. Said invention further comprising sustainable ecosystem elements encompassing a high pressure, symmetrical aeroponics and integrated multi-trophic aquaculture (IMTA) systems while also incorporating a microalgae bioreactor and organism reactor production system, digesters for waste management, raw food processing, post convenience food processing, packaging and integrated dry and cold storage facility all with robotic automation.

### BACKGROUND

The world trend is that there is less and less arable land available for agriculture which needs to feed more and more people, while still maintaining all the biological and other services that land and natural ecosystems provide. There is therefore an overwhelming imperative to produce quality food and biomaterial with high yields in the minimum possible space and feedstock material usage with the minimum possible ecological impact and near to the end use location.

There are multiple agricultural methods to process various plant species. Humans have been actively been processing plants since before recorded time. Aeroponics is one of those processes to grow plants in an air or mist type of environment without the use of soil or an aggregate medium which is also known as geoponics and commonly referred to as agriculture. The word “aeroponics” is derived from the Greek meanings to aero- (air) and ponos (labor). Aeroponics differs from conventional hydroponics, aquaponics, and in-vitro also known as plant tissue culturing growing methods. Unlike typical hydroponics, which uses a liquid nutrient solution as a growing medium that contains essential minerals to sustain plant growth, nutrients fed to the roots in a trough or liquid bed and generally immersed in a liquid based nutrient solution; or aquaponics which uses aeroponics or hydroponics and aquaculture as a symbiotic solution, aeroponics generally is orchestrated without a growing medium. Due to the fact that water is used in aeroponics to transfer plant nutrients; it is sometimes referred to as a type of hydroponics.

Aquaponics involves the symbiotic integration of the growth of aquatic species with growth of plants. The general concept of the aquaponics system is that the waste products from the aquatic species are used as nutrients for the plant species. In utilizing the nutrient-rich waste of the aquatic species, the plants somewhat cleanse the circulating water, making it suitable for the aquatic species to survive in.

The term horticulture refers to the processes associated with aeroponics plant species and plant products between the time plant species are germinated, grown and harvested, and the time the final product is delivered to the customer. The term in practice is extended to cover any aeroponics species harvested for commercial purposes, where the plant species are germinated, grown and harvested. There is an increasing demand for ready to eat aeroponics nutritional products such as pre-processed, pre-packaged organic salads, juices and smoothies. This would also entail products that don't need much preparation, commonly referred to as convenience foods for distributors, restaurants, retailers and end consumers.

The basic premise of aeroponics growing is to grow plants suspended in a closed and highly controlled environment by spraying the plant's roots and lower stem with an atomized or fine sprayed, nutrient-rich water solution. The leaves and crown, often referred to as the “canopy”, extend above the light limited root area. The roots of the plant are separated and isolated from potential light sources via the plant support structure. Prior art uses foam, rubber grommets and other media that would be compressed around the lower stem and inserted into an opening in the aeroponics support chamber, this decreases labor and expense; larger plants for example used trellising to suspend the weight of vegetation and fruit.

Aquaponics systems are being openly regarded and increasingly recognized as having the greatest potential to offer workable solutions to some of the world's greatest problems concerning modern agriculture and aquaculture systems. These problems include:
A) Unsustainable and increasing population growth, greatly effecting global potable water availability and usage in a world of quickly diminishing fresh water resources;
B) Land previously suitable for agriculture is becoming prohibitively expensive or unavailable due to urbanization and urban sprawl;
C) As a consequence of urbanization and globalization, the logistics of so called ‘food miles’ (‘food miles’ meaning the number of miles between food production, market, and the end consumer) is rapidly increasing, resulting in a range of ecological and human health problems—such as breakdown of nutrients, micro nutrient recycling, increased energy consumption and subsequent increased greenhouse gas emissions, loss of food freshness, loss of nutritional value, loss of visual appeal, etc.;
D) Waste effluents and chemical pollutants from commercial food production methods are causing ecological consequences and lasting human health problems, increased conservation land loss, loss of fertile soil from farmed soil runoff, nitrogen and phosphorous run-off, and water turbidity issues;
E) Net protein loss in conventional aquaculture from use of grain based crude proteins Which lack natural common food chain nutrients are increasingly used as aquaculture Feeds; and
F) Destruction of natural enzymes from over processing feedstock input
   Although aquaponics systems have the potential for realizing solutions to the these problems, prior art aquaponics systems have had very limited success actualizing, and more to the point, realizing overarching goals in this potential.

Presently, there is no shortage of food in the world, but it is the logistics of getting fresh, healthy food to the people effectively, economically, and without extended exposure to harmful bacterium and contaminants. Additionally, once a vegetable is detached from the plant and/or extracted for processing, it's no longer receiving nutrients after harvesting, as such it will immediately start to deteriorate, changing the color, taste, and texture of same while also allowing vitamin deterioration and mineral content depreciation. This greatly increases the need to produce high quality food locally and making same available to the market fresher, faster, and more safely.

Convenience food is commercially prepared for ease of consumption. Bread, cheese, salted food and other prepared foods have been sold for thousands of years. Other kinds have been developed and adapted in response to improvements in food technology. Types and availability of convenience foods can vary widely by country, geographic region and economics. Products designated as convenience food are often sold as hot, ready-to-eat dishes; as room-temperature, shelf-stable products; or as refrigerated or frozen food products that require minimal preparation such as typically just heating with a microwave, stovetop or oven.

Convenience foods have also been defined as foods that have been created to “make them more appealing to the consumer for quick, easy consumption, less messy cleanup. Convenience foods and fast goods are similar, because the development of both occurred to save time in the preparation of food. Both can cost less compared to the price of preparing the same foods from scratch when including bulk purchase prices.

In places such as the United States, increased food miles has resulted in the construction of tens of millions of square feet of both public and private cold storage facilities. These cold storage “super warehouses” will have huge freezers and coolers dependent on various refrigeration systems to control temperatures within the spaces to maintain product quality and freshness until shipped. With modern rack storage, it is not unusual to find product values of bulk product in excess of tens of millions stored at a single facility.

The preferred method for cold storage facilities may utilize multi-level, fully automated storage and retrieval systems using robotic labor force, automated inventory management and order processing fulfillment system, and allow for the smallest footprint (square feet) storing high volumes (cubic feet) of product for high efficiency standards. The primary concern of any facility is safety, security and preservation of its products stored within; although preservation of product through zones of required storage temperatures, especially when high values or highly susceptible to contamination are stored, requires rigid standard operating procedures to be carefully maintained.

From basic beginnings, ice houses helped develop the cornerstone of today's most modern refrigeration systems. They utilized design principles, materials of construction are controls that allow us to successfully store and distribute foodstuffs throughout the world marketplace. For extremely large cold storage facilities, preferred method of the current invention is the premiere choice because it produces the greatest cost effective net refrigerating effect (btu/lb), and the lowest brake horsepower per ton of refrigeration (BHP/TR) of any industrial refrigeration system.

In addition to the previously stated issues, temperature variations have altered ecosystems extensively. Although there was cooling experienced in particular seas and oceans, there is an overall warming trend throughout the world's fresh waterways, lakes, seas, and oceans—which, generally speaking, remains on a steady upward trend. The result of this trend is further global warming events in the oceans, seas, lakes, and waterways over the past few years as scientists suspect the changes are affecting plant and animal reproduction. The first six months of 2013 was characterized by new extremes in the physical and biological environment. Essential springtime blooms of plankton family organisms (which are a microscopic species that are the foundation of most aquatic ecosystems) are at the lowest global levels ever seen. This dramatic decline has also coincided with internationally recorded water surface temperatures that are the third-warmest on record, after an all-time high in 2012. Phytoplankton, the most basic form of plankton, are a massively important and a general necessity for the planet's overall ecosystems; they account for roughly half the organic matter produced on Earth, produce half the oxygen in the atmosphere, draw carbon dioxide out of the air, and serve as the foundational food source for most of the aquatic food webs.

The steep and rapid decline in the springtime plankton is also affecting the population levels of larger zooplankton, smaller aquatic invertebrate species that feed on the blooms. Scientists have discovered that rising ocean temperatures and increased acidic values has had a dramatic effect with altering the interaction and commutations of nutrients and organisms between different water columns of the various bodies and depths of water. As a result, fewer nutrients circulate from the lower water columns to serve as food for the phytoplankton in the upper water columns. Researches suspect this phenomenon was a major reason for a massive 40% decline that has been observed in global phytoplankton levels since 1950's. Is a known fact that roughly 90 percent of global warmings's total result goes into the heat trapping effect of warming the oceans.

Research additionally has demonstrated that the massive retreat and loss of arctic ice shelves are leading to earlier phytoplankton blooms in those regions of the ocean and beyond. The spring blooms are coming earlier, by as much as two months or more, than they were merely a decade ago. These changes pose serious risks for the collapse of larger food webs in the delicate ecological cycle, as the reproductive cycles of many aquatic and marine species are timed to the algae blooms.

Aquaponics is known and generally accepted as a controlled and isolated environment for cultivation of plants also known as aeroponics which is amalgamated with cultivation of aquatic species also known as integrated multi-trophic aquaculture (IMTA) (hereinafter referred to as “aquaculture”), which is species that live and grow in the water. Fish farming is a form of aquaculture where fish are raised in tanks and/or ponds for commercial food production purposes. Hydroponics is another form of aquaponics where plants are grown with its roots in flow channels or beds of liquids with mineral and vitamin nutrient enriched solution rather than soil.

Microalgae bioreactor and microorganism reactor production system is the primary method that is known and generally accepted as the most effective closed and isolated environment for production and culturing of microalgae and organisms. Prior art makes use of ocean, sea and freshwater based bioreactors and other various prior art microalgae and organism reactor growth systems, these aquatic based systems generally use a fresh water and/or brine and/or sea water pumped from surrounding water sources for water input, transfer and fluid circulation.

Prior art includes use of nutrients and crude proteins provided via pellets manufactured from processed grains for good and nutrients for aquaculture input, this food cycle is however greater in unhealthy excessive omega-6 to omega-3 ratio and higher percentage of low grade amino acid content yet deficient in several core vitamins, minerals, omega-3 and other natural enzymes that can be commonly found only in the natural food cycle. About half of this omega-3 fat is provided in the form of EPA (eicosapentaenoic acid) and a slightly lower amount if provided in the form of DHA (docosahexaenoic acid). The amounts of EPA and DHA contained in salmon are unusual among commonly-eaten foods. In addition to this high concentration of omega-3 fats, are the relatively small amount of omega-6 fats in salmon; thus its outstandingly healthy ratio of omega-3 to omega-6.

For example, four ounces of salmon typically will contain less than ½ gram of omega-6 fat, for an omega-3 to omega-6 ratio of approximately 5 to 1, this is a healthy ratio. The average U.S. diet, the ratio has repeatedly been shown to be lopsided in a highly unhealthy direction, with at least 4-5 times as much omega-6 fat as omega-3 fat, and in some studies, ratios of omega-6 to omega-3 is up to 10-20 times or more which systematically increases affliction with autoimmune disease, inflammation related issues and illnesses, bowel syndrome disease, and a host of other diseases affecting critical organs such as the heart, kidney, liver, and brain. In our World's Healthiest Foods rating system for food, only two other foods provide more omega-3s per standard serving than salmon. Those two foods are walnuts and flaxseeds. Both of these plant foods are outstanding sources of omega-3s. They are however unable to be compared on an equal basis to salmon because their omega-3 fats come in the form of alpha-linolentic acid (ALA) which cannot be readily digested when in concert with excessive omega-6 intake unlike direct intake of EPA or DHA.

Other prior art of aquaculture and aquaponics systems rely on commercially produced fish meal pellets as their feed source. Fish are typically harvested from the sea, processed into fishmeal as dry pellets and then fed to aquaculture or as input to aquaponics farms. Current contaminations levels of harmful toxins and heavy metals from aquatic species harvested in the wild and from pen raised species have cased warnings about unhealthy consumption limits, issued from many health agencies world wide due to the dangers of harmful contaminant consumption risk involving many dangerous heavy metals such as mercury and POPs (including dioxins; dioxin-like compounds, or DLCs; and polychlorinated biphenyls, or PCBs). An additional unfortunate consequence of harvesting fish meal from the wild results in less fish harvested as an end product than was originally extracted from the ocean to product the feed pellets, i.e. a net loss of protein and cause for imbalance and great harm to the oceans, seas, lakes, and waterway ecosystems. From a sustainability and environment viewpoint, this is ecologically inefficient as well as expensive and unsustainable.

Often overlooked, omega-3 content of harvested fish is essential for balanced non-ruminant diets. The present invention generally yields very high quality aquaculture products that are naturally high in omega-3 content and lower with omega-6 content. The majority of commercially prepared fish feeds are made primarily from cheaper protein sources such as corn, soybeans and other nitrogen rich protein sources. Prior art using grain feed base versus fish meal methods, yields aquaculture products much higher in an unhealthy balance of omega-6 to omega-3 fatty acids ratio.

One solution to mitigate the problems of variable climatic effects is to use a greenhouse For plant life cycle and enclosed aquaculture system for aquatic species life cycle to enhance and extend the grow periods and to protect the aquaponics system from the environmental and climatic driven events. However, large scale commercial aquaponics systems are exceptionally operational expensive from its energy usage and very capital intensive in terms of the cost of the aeroponics/hydroponics system, aquaculture system, purchasing land, land preparation, climate and environmental control, pest control, water storage, water treatment, irrigation systems, nutrient mixing systems, harvesting and standby energy systems.

A majority of usable space is generally wasted in a typical large greenhouse, mainly due to lighting considerations and lack of plant to building area ratio and plant density. Therefore a lot of energy may be needed to control the climate of unused and area that has little or no benefit within or of the greenhouse. The use of artificial lighting may be an option, but requires high energy consumption resulting in high energy bills leading to higher production costs. Plant production and harvesting in greenhouses can also be very labor intensive.

Typically, agricultural produce is cultivated in rural areas with its sample availability of fertile soil and planting surface area while the majority of consumers are concentrated in urban areas. Thus, there is a high cost of logistics in transporting such agricultural produce to the market, potential of wasting and product deterioration awaiting transport and during transport—all of which can take several days. Aeroponics is a technology for growing plants in a nutrient solution with or without the use of artificial medium to provide mechanical support. Aeroponics systems in temperate regions of the world are enclosed in greenhouse type structures to provide temperature and humidity control, reduce evaporative water loss, and to reduce and potentially eliminate disease and pest infestations.

Energy input is required for heating and cooling of thermal applications to maintain the proper temperature of the aquaculture and aeroponics systems, energy is needed to maintain many other critical environmental aspects of the aquaponics system. Energy is also required to continuously pump and circulate water from the aquaculture facility to the aeroponics system and back to the aquaculture facility. Energy is required for operation of the atmospheric air ventilation fans for environmental control, compressors and pressure swing absorption units for aeration to maintain required dissolved oxygen levels to promote aquaculture species health quality growth. Energy in the form of electricity, heating and cooling is a very major input requirement; this has been found to be one of largest primary expenses which are expended to run a conventional aquaponics system.

In response to energy costs there is a general need to reduce the power consumption of light sources while maintaining their ability to stimulate the desired species plant growth. Further advantage is achieved by targeting the lowest energy consumption device while maintaining optimized photosynthetically active radiation (PAR) at wavelengths that are specific to each individually specific plant species through the use of adaptive biometric and thermal imaging analysis, monitoring and control. Further advantage is achieved by targeting a light source able to withstand the humidity and aerosol water droplets commonly found in aeroponics environments. Further advantage is achieved by targeting a light source with needs little or no maintenance for extended operational depreciation and lifetimes.

Other sources of light, for example light emitting diodes (LEDs), are known to be capable of producing useful PAR with relatively small power consumption, virtually no heat, and very long life. Therefore, these other sources of light, for example LEDs, can be adapted as grown lamps to offer a solution to the high power consumption of high intensity lamps.

Another disadvantage of current high intensity discharge lamps (HID) is that they produce light by electrically arcing open current between an anode and cathode for the purpose of heating of high pressure gasses to a state of excited black body emission. This is essentially the same primitive principle resulting in the orange glow from an electric range element, except in that case the electricity stays safely within the heating element. Related issue is that much of the power released in an arc lamp is emitted as photons which move indiscriminately of direction. Furthermore, the energy released falls largely in bands of the light wave spectrum that are not useful for the stimulation of specific plant species growth. There is evidence that the light energy emitted by such systems may be detrimental to various stages of plant development which are not directly involved in perennial harvest cycle.

Contrarily, LEDs are advantageous in the lighting's ability to simulate these environmental signals for existing natural plants species, also useful for sending specifically calculated and programmed signals based on an adaptive learned responses to specific species needs through adaptive biometric and thermal imaging analysis, monitoring and control. Further advantage is achieved by LEDs ability to simulate these environmental signals by continuous control that is able to vary the amplitude of the power outputs of multiple bands of select phototrophic radiation specific to individual species requirements. The greatest available light source spectrum and intensity is placed into those bands which feed photosynthesis. For example light source spectrums of approximately 450 nm-470 nm and approximately 640 nm-670 nm. There is however, other special light source spectrums bands have been required for such environmental signals as diurnal cycles (approximately @730 nm), seasonal cycles (approximately @600 nm), and competitive cycle (approximately @525 nm). There are other special light source spectrum bands in the ultraviolet range which is the ultraviolet related frequencies of environmental signals between approximately 360 nm-410 nm. These light source spectrums may trigger existential quantifiers in a specific plant species life.

It is well known and generally accepted that proper illumination is the key ingredient along with proper nutrient balance in promoting and maintaining robust and healthy plant growth. The present invention through the use of artificial intelligence and machine learning with the inclusion of adaptive biometric and thermal imaging analysis, monitoring and control the illumination of optimized spectral light sources outputs can be achieved to meet the specific needs of various plants during their highly independent and very specific species dependent growth phases. Known light sources are extremely energy intensive and as most are adapted for delivering a very high lumen output. Wattages of these high intensity arc-tube lamps range from 250 W to 1250 W. Generally, in commercial aeroponics and horticultural applications many of These lamps may be required for operation of a facility. It can be readily observed that the aggregated power consumption of these types of light sources in a commercial operation setting is a very large part of operational expense. A great degree of electrical energy consumption of a high intensity discharge lamp (HID) is lost in the form of wasted heat.

Another disadvantage of current high intensity discharge lamps (HID) is that they produce light by electrically arcing open current between an anode and cathode for the purpose of heating of high pressure gasses to a state of excited black body emission. This is essentially the same primitive principle resulting in the orange glow from an electric range element, except in that case the electricity stays safely within the heating element. Related issue is that much of the power released in an arc lamp is emitted as photons which are directionally indiscriminately. Furthermore, the energy released falls largely in bands of the light wave spectrum that are not useful for the stimulation of specific plant species growth. There is evidence that the light energy emitted by such systems may be detrimental to various stages of plant development which are not directly involved in perennial harvest cycle.

Ideally, the closed environment is to keep the system free of pests and disease so that the plants may grow healthier and more quickly than plants that grown in a medium. However, most prior art aeroponics environments are not entirely perfectly closed off to the atmosphere, thus pests and disease may still cause a threat. Controlled environments advance plant development, health, growth, flowering and fruiting for any given plant species and cultivars.

Due to high sensitivity of root systems, aeroponics is combined with a high pressure backup aeroponics spray system, which is available for use as an emergency “crop saver” for backup nutrition and water supply in case the primary aeroponics spray system failure. Commercial aeroponics systems incorporate hardware features that accommodate the crop's expanding root systems. Water and nutrient hydro-atomization commonly used in high pressure aeroponics equipment involves the use of sprayers, misters, foggers, or other devices to create a fine mist of solution to deliver nutrients to plant roots. Aeroponics systems are normally closed-looped systems providing macro and micro-environments suitable to sustain a reliable, constant air culture. Numerous inventions have been developed to facilitate aeroponics high pressure spraying misting. The key to healthy root development in a high pressure aeroponics environment is the size of the water droplet. In commercial applications, a hydro-atomizing spray is employed to cover large areas of roots utilizing high pressure misting. A variation of the mist technique employs the use of ultrasonic foggers to mist nutrient solution in low-pressure aeroponics devices.

Modern aeroponics allows high density companion planting of many food and horticultural crops without the use of pesticides. Aeroponics is an improvement in artificial life support for non-damaging plant support, seed germination, environmental control and rapid unrestricted growth when compared with hydroponics and drip irrigation techniques that have been used for decades by traditional agriculturalist.

Larger aeroponics processing companies often operate their own aeroponics production, harvesting and distribution warehouse operations. The products of the aeroponics industry are generally sold to grocery chains and/or to distribution intermediaries. Aeroponics species are highly perishable. The central concern of aquaculture processing is also just as important for aeroponics to prevent aeroponics products from deteriorating, and this remains an underlying concern during other processing operations.

Aeroponics plant processing can be subdivided into aeroponics product handling (which is the preliminary processing of raw, freshly harvested products), packaging of aeroponics products, and, finally, into convenience food processing of products such as salads, juices, smoothies and herbal mixes product lines or a host of additional types and flavors of finishing processing. Vegetable, herb, and flower processing can be incompatible with each other because of ethylene gas, which causes ripening. Fruits give off this gas, while vegetables are extremely sensitive to it. Isolating product lines is extremely important to avoid untimely deterioration and demise of the sensitive vegetables and other aeroponics products. The following are a few examples of some ethylene-producing fruits: cantaloupe and tomatoes. The following are a few examples of a few ethylene-sensitive veggies: asparagus, broccoli, cabbage, carrots, cucumbers, green beans, and lettuce.

Vegetables that do particularly well in the freezer include asparagus, broccoli, peppers, spinach, sweet corn and squash, while vegetables that contain a lot of water, like cucumbers and lettuce, will store very poorly in the extreme cold. Root vegetables can last for months when stored properly, making them a wonderful fresh option to plant and store in bulk to offset when other products are needed to be grown.

Aeroponics plants are greatly affected by exposure temperatures. Refrigeration is normally set between 36 and 38 degrees Fahrenheit (2.2 and 3.3 degrees Celsius) to keep food fresh but not frozen, which can damage and potentially destroy the product. Some aeroponics products such as potatoes, onions, squash and garlic need cool temperatures but also require protection from light and are stored in dark yet cool settings.

Another natural subdivision is the primary processing involved in the peeling, separating and freezing and drying of fresh aeroponics products for onward distribution to fresh product retailers and specialized catering outlets, and the secondary processing that produces chilled, frozen, boxed, sealed and/or enclosed plastic packaging and canned products for the retail and specialized catering trades.

When aeroponics products are harvested for commercial purposes, they initially need some pre-processing to be readied safely for delivery to the next part of the product process chain in a fresh and undeteriorated condition. Typical handling and processes are transferring the aeroponics from grow out beds to the transfer systems to holding areas in the processing area. Further processing and handling may commence such as sorting and grading, peeling, separating, freezing and chilling, storing the chilled aeroponics species. The number and order in which these operations that are undertaken differ with the various aquaculture species and the type of processing needed for the finished product.

Aeroponics product preservation techniques are required to prevent aeroponics product spoilage, reducing waste from product handling and lengthen shelf life. There are processes designed to inhibit the activity of spoilage bacteria and/or enzymes and the metabolic changes that result in the loss of aeroponics product quality. Spoilage bacteria are the specific bacteria that produce the unpleasant odors and flavors associated with spoiled aeroponics product. Aeroponics normally host a variety of bacteria that are not spoilage type of bacteria, and most of the bacteria present on spoiled aeroponics product played no basis in the spoilage. For, a bacterium to initiate and flourish, it requires the right temperature, sufficient humidity and oxygen, and surroundings that are pH balanced but not too acidic. Various preservation techniques work by interrupting one or more of these requirements.

Accordingly, it would be advantageous of the present invention aquaponics system is devised in which may at least partially address the problems above and to provide the public with a useful commercial alternative. The principal advantages of the preferred method of aeroponics is incorporation of a STRict Environment Aeroponics Management (STREAM) monitor, analysis and control system that culminates in ultra-high density production techniques to maximize yields.

The primary advantage of aeroponics (STREAM) when compared to filed grown produce is the isolation of a crop from the soil, eliminating soil related contact contaminations, a virtual indifference to ambient temperature and seasonality, highly efficient use of water and nutrients, minimized use of land area, and suitability for mechanization and robotics, enhanced disease and pest control. A crop produced in soil also suffers from potential diseases, pests, salinity, poor structure and drainage. However, traditionally hydroponics and/or systems required high initial capital costs and introduced bacterial issues from light exposure to the fluid bed channels and increased risks such as root rot.

In prior art the following designs and configurations are well known for providing energy generation using various types of fuel, chemical, and thermal sources: aquaponics, aeroponics, aquaculture, bioreactors, cold storage, dry storage, fast freeze systems, wind turbines, solar generators, thermal solar, photovoltaic solar, chemical and thermal energy storage, stirling applications and processes, chillers, refrigeration, heating and air conditioning, water heating, distillation, water purification and desalination systems, as well as electrical regeneration systems. It is envisioned that the electrical wiring, liquid, semi liquid, and solid material transfer conduits may consist of conduits, ducts, pipes, hoses, pneumatic tubing conveyer belts, or any means of connecting loops and circuits, conveying solid and/or semi-solid matter. Specifically, U.S. Pat. No. 2,732,663 to Dewey covers A SYSTEM FOR PHOTOSYNTHESIS and describes a system for conducting photosynthesis in which conduits are utilized for liquid and gas. Wherein said conduits comprise of long, thin-walled, flexible, translucent tubes.

However, prior art of the above systems and devices, particularly when said referenced inventions are physically deployed, they are generally not planned, established or orchestrated to benefit from higher efficiency as integral components as elements in an integrated multi-level control system environment by forming a complete and essential logical cycle or in otherwise would be referred to as an energy ecosystem, generally systems are planned for a deployment with an efficiency basis as an independent device with subpar system design performance.

Deployment of prior art had required higher part count, increased manufacturing costs, increased assembly cost, increased transportation cost, increased subpart count and more costly parts with larger custom parts inventory required, overlapping and duplicated subsystems, frequent problematic maintenance and repair costs, rising levelized cost of energy and products production, causing higher operating expenses, grid energy connection and transfer line losses.

Wind energy technology is typically used to convert kinetic energy from wind into mechanical energy and/or electricity. To extract wind power, a wind turbine may include a rotor with a set of blades and a rotor shaft connected to the blades. Wind passing over the rotor connected blades may cause the blades to turn and the rotor shaft to rotate. In addition, the rotating rotor shaft may be coupled to a mechanical system that performs a mechanic task such as pumping water, atmosphere gas separation compressors, providing rotational energy to generate electricity.

Alternatively, the rotor shaft may be connected to an electric generator that converts the rotational energy into electricity, which may subsequently be used to power a consumer, commercial or industrial device, and/or electrical grid.

Solar energy technology is typically used to convert radiated light energy from the sun into thermal energy and/or photovoltaic electricity. to extract solar power, a collection surface and/or reflector as is the case with thermal solar technologies to concentrate the solar energies on the aforementioned solar collector surface. Solar energy striking the collection surface is converted into photovoltaic generated electrical energy or as thermal generated heat for direct use, transfer and/or storage.

However, the variable nature of wind and availability of solar energy may interfere with base-load and/or on-demand generation of electricity, generated products and by products from wind and solar energy. For example energy storage using chemical and thermal techniques may be required to offset fluctuations in electricity, products and byproducts generated from wind and solar power and/or maintain reliable electric and or thermal energy provisioning service and/or in a private and public electrical grid.

Aquaculture, also known as aquatic farming or aquafarming, is the farming of aquatic organisms such as fish, crustaceans, mollusks and other aquatic species. Aquaculture may involve cultivating freshwater and saltwater populations under highly controlled conditions, and can be contrasted with commercial fishing, which is the harvesting of wild aquaculture and farmed aquaculture. Farming implies some from of intervention in the rearing process to enhance production, such as regular stocking, feeding, protection from predators, etc. Farming also implies individual or corporate ownership of the stock being cultivated.

Particular kinds of aquaculture include fish farming, shrimp farming, oyster farming, and the cultivation of ornamental fish. Particular methods include aquaponics and integrated multi-trophic aquaculture, both of which integrate aquaculture farming, plant farming and biomass species enhanced environments.

Integrated multi-trophic aquaculture (IMTA) provides the by-products, including waste, from one aquatic species as inputs (fertilizers, food) for input to another species cycle. Farmers combine fed aquaculture such as fish, shrimp and crawfish with inorganic extractive such as microalgae and micro-organisms and organic extractive such as mollusks and filter feeder aquaculture to create a highly balanced systems for environmental remediation via bio-mitigation, economic stability (improved output, lower cost, high efficiency, diverse product offering diversification, risk reduction and mediation) and global social acceptability using the best environment management practices.

Selecting appropriate species and adequate sizing of the various species populations to provide necessary ecosystem balance will allow the biological and chemical processes involved to achieve a sustainable balance, while mutually benefiting the organisms and improving overall ecosystem health. In an ideal world, amalgamated cultured species which each may yield valuable commercial “crops” products and byproducts would exist. IMTA can synergistically increase efficiency of total output while reducing input requirements.

The aquaculture farming of aquatic species in tanks is the preferred and commercially accepted method. Some of the most important and greatest amount of aquaculture raised worldwide, some of the most important aquaculture species used in aquaculture farming are shrimp, prawn, crawfish, carp, salmon, trout, bass and tilapia, oyster, mussel and clam, mollusks and catfish.

Aquaculture is highly perishable foodstuff which needs proper handling and preservation to have a long shelf life and retain a desirable quality and nutritional value. The central concern of aquaculture processing is to prevent aquaculture from deteriorating which leads to excessive waste removal and product loss. The most obvious method for preserving the quality of fish is to keep them alive until they are ready for cooking and eating. For thousands of years, China achieved this through the aquaculture of carp.

Humans have been processing fish since early Neolithic times. The term aquatic processing refers to the processes associated with aquatic species and aquatic products between the time aquatic species are caught of harvested, and the time the final product is delivered to the customer. The term in practice it is extended to cover any aquatic organisms harvested for commercial purposes, whether caught in wild fisheries or harvested from aquaculture of aquatic species farming. There is an increasing demand for ready to eat aquaculture products, or products that don't need much preparation, commonly referred to as convenience foods for distributors, restaurants, retailers and end consumers.

Larger aquatic processing companies often operate their own aquatic production, harvesting and distribution warehouse operations. The products of the aquatic industry are generally sold to grocery chains and/or to distribution intermediaries. Aquatic species are highly perishable. A central concern of aquaculture processing is to prevent aquaculture from deteriorating, and this remains an underlying concern during other processing operations.

Aquaculture processing can be subdivided into aquaculture handling. Aquaculture handling can include the preliminary processing of raw aquaculture, manufacture of aquaculture products, as well as processing into convenience food. Aquaculture processing can include products such as garlic prepared shrimp and/or salmon, Cajun catfish, breaded product lines, and/or a host of additional types and flavors of finishing processing. Further processing and handling may include sorting and grading, peeling, deveining, deheading, skinning, bleeding, gutting and washing, chilling, storing the chilled aquaculture species. The number and order in which these operations that are undertaken differ with the various aquaculture species and the type of processing needed for the finished product.

Control of temperature with the use of ice preserves fish and extends shelf life by lowering the temperature. As the temperature is decreased, the metabolic activity in the fish from microbial or autolytic processes can be reduced or eliminated. This is achieved by refrigeration where the temperature is dropped to about 0° C. or freezing where the temperature is dropped below −18° C.

An effective method of preserving the freshness of aquaculture is to chill with ice by distributing ice uniformly around the aquaculture, preferably in slurry consisting of ice and water. It is a safe and highly benign method of cooling that keeps the aquaculture suspending in moisture and in an easily stored forms suitable for transport. It has become widely used since the development of absorption and mechanical refrigeration, which makes ice easy and cheap to produce. Ice is produced in various shapes; crushed ice and ice flakes, plates, tubes and blocks are commonly used to cool aquaculture.

Particularly effective is when ice is used in a slurry, made from micro crystals such as those made with injection of aeration to initiate the formation of crystals of ice formed and suspended within a solution of water and freezing point depressant, such as the addition of salt. New methods include pumpable ice technology. Pumpable ice flows like water, and because it is homogeneous, it cools the aquaculture faster than fresh water solid ice methods and eliminates freeze burns. It complies with various protocols such as HACCP and ISO food safety and public health standards, and uses less energy than conventional fresh water solid ice technologies.

Targeted species preservation techniques are required to prevent product spoilage, reducing waste from product trimming and lengthen shelf life. There are processes designed to inhibit the activity of spoilage bacteria and metabolic changes that result in the loss of product quality. Spoilage bacteria are the specific bacteria that produce the unpleasant odors and flavors associated with spoiled product. Targeted species will normally host a variety of bacteria that are not spoilage type of bacteria, and most of the bacteria present of spoiled product played no basis in the spoilage. For, a bacterium to initiate and flourish, it requires the right temperature, sufficient humidity and oxygen, and surrounding that are pH balanced but not too acidic.

Conventional aquaculture farm production systems are of four main types, namely a pond based system, a cage system, a raceway production system and a tank based system. In pond production fish are stocked in growing ponds. There are three primary methods: (a) fish nutrients are based on plant and aquatic life available in the pond (typical yields of 200 kg/hectare); (b) the ponds are fertilized (typical yields of 1-2 ton/hectare); or (c) ponds are fertilized and the fish are also fed high grade food (typical yields of 2-12 ton/hectare)

This type of fish farming is a batch process. Eventually the pond water becomes unsuitable for fish production over time and can be contaminated via airborne contaminates, run off and droppings from birds. Once contaminated it has to be replaced, either naturally (rain, snow, etc.) or by mechanical pump methods. Generally the best yields achievable are 1 kg of fish per ton of water.

In cage fish farming, fish are held in cages floating in a large body of water (lake or sea) and the fish are fed nutrient complete diets. Fish waste uses gravity to drop through the mesh bottoms of the cages. This technique relies on the large body of water mass to dilute the water in the cages to maintain suitable nontoxic growing conditions. Yields based on cage area can be quite substantial, 100 tons per hectare.

In raceway systems aquaculture are grown out in raceways and are fed nutrient complete diets. Fresh water is continuously pumped to pass through the raceway to remove waste and to maintain suitable nontoxic growing conditions. Yields are based on the area of raceway, can be up to 400 tons per hectare, however, this is based on water requirements for example (265 cubic meters of water per hour, per ton of a certain fish species.

In an aerobic system, such as composting, the microorganisms access free, gaseous oxygen directly from the surrounding atmosphere. The byproducts of an aerobic process are primarily carbon dioxide and water which are the stable, oxidized forms of carbon and hydrogen. In an aerobic system the majority of the energy in the staring material is released as heat by their Oxidization into carbon dioxide and water.

Digester unit systems typically include organisms such as bacteria and fungi that are able to break down lignin and celluloses to a greater extent than aerobic bacteria. Due to this fact it is possible, following anaerobic digestion to engage composting using aerobic digesters allowing further volume reduction and stabilization.

With some raceway systems, waste water is treated and then recirculated for reuse. Treatment of the waste involves passing the waste through aerobic digesters (sometimes called “active filters”) and oxygenation. This treatment is sufficient to reduce ammonia and nitrites to less toxic nitrates, but eventually the water becomes unsuitable and unhealthy for aquaculture growth which then has to be replaced.

Biomass can also build up in the digestion units. In the last few years there has been an increasing interest in using plants as a means of water treatment (applied for use in the fields of aquaponics). In this technique, the plants, e.g. tomatoes, lettuce, etc. are grown hydroponically with wastewater from the fish being used as the hydroponic solution. The treated water from the aeroponics bed is then recycled back to the aquaculture tanks. Additionally, some of the aeroponics plant produce can also use as a food supplement as nutrient input for aquaculture. Aquaponics is in its infancy as a science and is yet to be widely applied on a global basis.

Fulfilling the global demand and commercial interest in aquatic organisms in general and in marine invertebrates with its particular ability of many of these organisms to produce natural compounds that may be used also used in the pharmaceutical and cosmetic industry. These compounds are usually secondary metabolites, which are produced in low amounts by the organism. Development of these natural compounds for input to aquaculture, pharmaceuticals and cosmetics is often hampered by a supply problem: there is not enough biological material available to complete the development research. Many aquatic organisms are rare and difficult to collect on a large scale. In addition, many aquatic organisms are difficult to culture efficiently on a large scale. This is in particular the case for filter feeding invertebrate animals. These are animals that feed on suspended particulate organic matter and on dissolved organic matter that is filtered out of the surrounding water.

Past prior art used various methods, most existing methods culture these animals in sea-based aquaculture: growing the animals in their natural environment. Due to unpredictable and uncontrollable circumstances such as contamination, rising acidic pH levels of the seas and other issues that prevail outdoors in open atmosphere aquaculture is a technique and/or method with extremely high risk factors. It is also fairly difficult to find a location that is suitable for setting up a viable aquaculture system without subjecting cultures to uncontrollable factors such as diseases, variable temperatures, losses due to damage, etc.

Aquaculture of filter feeding invertebrates can be greatly improved by growing such cultures nearby sources and locations that are enriched with nutrients, such as areas around and near closed cycle environmentally controlled aquaculture systems.

A large variety of land-based systems have been utilized to culture fish, shrimp and several of other marine animals. Some of the prior art devices and methods described in literature consist of two or more separate enclosures for pre-treatment of the water that is used in the system and/or biofiltration (cleaning) of the water in the system.

Land-based systems also have the advantage that extra nutrients can be efficiently provided to the animals in the culture. Common aquaculture feeds are usually solid, visible particles, for instance dried foods or cultured mesozooplankton. This food is suitable for animals with eyes and locomotive capabilities such as fish and shrimp, which can actively ingest these larger food particles. It is not suitable to most filter feeders, which are sessile and feed upon microscopic particles.

Some systems use the natural food chain to feed the animals in the culture. In these so-called “Greenwater systems” for growth of algae (both macroalgae and microalgae) is augmented by providing nutrients such as phosphate and nitrate to the water in the system. These systems are not suitable for the high-density culture of filter feeders.

### SUMMARY

This Summary is provided to introduce a selection of concepts in a simplified form that are further described below. This Summary is not intended to identify key features or essential features of the claimed subject matter, nor is it intended to be used to limit the scope of the claimed subject matter. Additionally, the claimed subject matter should not be limited to embodiments that solve and disadvantages noted in any part of this disclosure.

Disclosed herein is an aquaponics systems comprising at least one of each of the following: (a) atmospherically sealed aeroponics unit containing at least one plant species; (b) atmospherically sealed aquaculture unit; (c) atmospherically sealed microalgae bioreactor unit; (d) atmospherically sealed microorganism reactor unit; (e) atmospherically sealed filter feeder unit; (f) atmospherically sealed filter unit; (g) atmospherically sealed digester unit; (h) a biometric camera unit; (i) a thermal camera unit; and (j) a control unit. The aforementioned control unit comprises a computer having a first processor and a first machine readable medium storing instructions providing at least one of the following: (a) artificial intelligence; (b) machine learning system; (c) computer interfaced adaptive metrics; and (d) biometrics and thermal analysis system. In addition, the control unit provides facility control instructions and is connected through a network to execute commands automatically from said artificial intelligence or through manual intervention by a user.

Furthermore, provided by the aquaponics system of the current invention is a method for operating an aquaponics water supply. The method comprises providing water to at least one aquaculture unit from one or more water storage units. The water provided to the aquaculture unit is then provided to one or more aeroponics units wherein said water passes through at least one biofilter, filter feeder, and digester before it reaches the one ore more aeroponics units. The system also allows for the recapture of atmospheric water from plant transpiration in the one or more aeroponics units and returning the recaptured water to at least one water storage unit.

The aquaponics system of the present invention may additionally comprise of at least one of each of the following: (a) an atmospherically sealed processing unit; (b) atmospherically sealed packaging unit; (c) an atmospherically sealed dry storage unit; (d) and an atmospherically sealed cold storage unit. Additionally, the aforementioned one or more digester units can comprise of an anaerobic and/or aerobic type digester. The aquaponics system may further comprise of a renewable power unit wherein said power unit provides heating, cooling refrigeration for cold storage, and electricity to operate the aquaponics system of the current invention. Moreover, the one or more aeroponics units may further comprise at least one of each of the following: (a) germination area; (b) grown area; (c) water and nutrient storage area; and (d) finishing area. The previously mentioned grow area may comprise of one or more levels in which plants may be placed, for example in an A-frame ladder structure, to increase the efficiency of the space within the grow area. The one or more aquaculture units may further comprise at least one of each of the following: (a) pisciculture area; (b) grow out area; (c) pre-processing area; (d) processing area; (e) preparation area; (f) chilling area; and (g) storage area. The one or more filter units may further comprise of a least one of each of the following: (a) mechanical filter; (b) biological filter; (c) anaerobic digester; and (d) aerobic digester.

Additional benefits are achieved by allowing the aquaponics system of the current invention to control one or more of illumination, heating, cooling, humidity, nutrient mix, gas composition, and nutrient supply via the control unit. Further efficiency is achieved by collecting water from at least one of heating, cooling humidity control, and one or more digester and utilizing said water for the previously-referenced nutrient supply. Furthermore, the control unit analyzes, identifies, monitors, tracks, and records all species, including but not limited to plants, animals, microorganisms, and insects, through one or more camera, biometric camera, and thermal camera units. By monitoring all species within the system, the control unit is further able to customize and control nutrient supplies to each species within said system as necessary. Further, the control unit can act as a security system by alerting administrators of unauthorized access and/or problem areas within the system. The control unit may also interface with one or more robotic apparatus that allow the control system to transfer species from one sub-unit area to another, i.e. transfer of seeds from a germination area to a grow area after proper germination occurs, as well as with harvesting, processing and other general automation duties of the system.

The aquaponics system of the current invention, in its preferred embodiment, may also comprise of the ability to treat water through nitrification by way of a nitrification entity which comprises of at least one tank separated from the plant growing apparatus. They aquaponics system may also further comprise an insect larvae production module wherein said module comprises a reversibly sealable container for housing organic waste and an insect larvae outlet pipe. Additionally, the above-reference bioreactor may comprise of one or more enclosures to accept enriched water. The one or more enclosures allow enriched water to be aged for pre-determined time periods for use within the system.

Also provided is an apparatus for metabolism manipulation utilizing at least one illumination array and at least one programmable microcontroller. The microcontroller is connected to said illumination array and is also be connected to the above-mentioned control unit and allows for manual and automatic local and remote control of said illumination array. The illumination array comprises of one or more plurality of light sources that have one or more respective light spectrum emissions. The aforementioned apparatus allows for the determination of specific photosynthetic properties of one or more plants species, utilizing one or more plurality of light sources with light emissions that are compatible and/or complimentary to said specific plant species and placing said plant species under said one or more plurality of compatible light sources wherein said light sources are controlled by at least one connected microcontroller. Additionally, the at least one microcontroller connected to the illumination array allows one or more plurality of light sources to simulate a pre-dawn and/or after-sunset glow as well as allowing the one or more plurality of light sources to flash and/or light for specific intervals, times, periods, and illumination intensities.

The combination of the above systems increases efficiency and the above-identified control unit reduces or eliminates the need for constant human intervention and monitoring while also improving the genetics of all species within the system. The improved genetic characteristics of species within the system can lead to improved disease resistance, as well as reproduction and growth rates of said species.

Therefore, objectives of the present invention include, but are not limited to:

One object of the present invention is to greatly enhance the localized food and biomaterial production by utilizing localized renewable energy generation and localized energy storage for on demand availability; thereby lowering expensive commercial grid energy metered use.

A second object of the present invention is to provide a production facility that is based on symbiotic relationships with optimized emulation of the natural food cycles.

A third object of the present invention is to decrease transportation requirements for food production while increasing per unit efficiency of said food production capabilities for local producers.

In addition to the above objectives, the following are also objectives that include improvements based on U.S. patent application Ser. No. 14/081,271, filed Nov. 15, 2013 entitled HYBRID TRIGENERATION SYSTEM BASED MICROGRID COMBINED COOLING, HEAT, AND POWER PROVIDING HEATING, COOLING, ELECTRICAL GENERATION AND ENERGY STORAGE; and paten Cooperation Treaty Application No. PCT/US13/70313, filed Nov. 15, 2013 entitled HYBRID TRIGENERATION SYSTEM BASED MICROGRID COMBINED COOLING, HEAT, AND POWER PROVIDING HEATING, COOLING, ELECTRICAL GENERATION AND ENERGY STORAGE, the entire disclosure of each of the above application is hereby incorporated by reference.

A fourth object of the present invention is to provide a device wherein multiple components may be associated and interconnected with applications to one another to enhance efficiency and power production capabilities. This is effectuated by combining element processes to reduce losses by combining device element cycles and applications of material usage, thermal, and electrical energy electrical demands.

A fifth object of the present invention is to reduce system component non-beneficial and redundant manufacturing and construction material requirements.

A sixth object of the present invention is to reduce system components count and area use requirements and greatly increases the ratio of production generated; with consideration to system component install costs further than previously possible, due to the improvement of hybrid integration and generation.

A seventh object is to enable high efficiency by enabling thermal storage for heat and cold storage, providing for on demand availability versus prior art usage of inefficient usage by increased startup and shutdown energy requirements of generation on demand of individual component applications and processes.

An eight object is the inclusion of energy generation, storage, component, and area cooling and/or heating requirements into a single system solution; recycling thermal energy from other processes waste heat to enhance efficiency and reduce system energy input requirements.

A ninth object is to recycle generated waste heat energy to use stored water supplies in closed loop coolant system to reduce subsystem requirements and maintenance.

A tenth object is to recycle generated waste heat for ground water and waste water reclamation and purification while reducing input energy requirements.

An eleventh object is to recycle generated waste heat for potential use in desalination while reducing input energy requirements.

A twelfth object is to recycle regenerated waste heat for use in distillation while reducing input energy requirements.

A thirteenth object is to recycle regenerated waste heat for use as replacement for thermal processing of water, for heating water for usage, and storage for on demand availability while reducing input energy requirements.

A fourteenth object is to provide potable water from localized, unprocessed water sources or contaminated public water provisioning.

A fifteenth object is to store thermal energy to enable scalable commercial mass energy storage.

A sixteenth object is to use locally generated biomaterial as localized input for higher level product production.

A seventeenth object is to use stored thermal energy for conversion into localized thermal application use for on demand availability and usage.

An eighteenth object is to use stored chemical energy for conversion to electrical and/or thermal energy.

A nineteenth object is to reduce the carbon footprint for electrical and thermal generation.

A twentieth object is to reduce the carbon footprint for localized energy consumption.

A twenty-first object is to enable a localized renewable energy ecosystem for generation, storage, and regeneration.

In addition, other objectives will be apparent from the figures and description herein below.

### BRIEF DESCRIPTION OF THE DRAWINGS

FIG. 1 is a schematic block diagram of the Aquaponics System of the present invention illustrating the primary facility of the preferred embodiment of the invention and highlighting particular interconnected elements thereof.

FIG. 2 is a schematic block diagram of the bioreactor, reactor, filter feeder, fish meal, and biomaterial process modules in accordance with the preferred embodiment of the present invention.

FIG. 3 is a schematic block diagram of the Aquaponics System of the present invention illustrating the preferred embodiment of the Aquaculture Process and corresponding modules including processing, packaging, and storage processes.

FIG. 4 is a flowchart illustrating the Aeroponics Process in accordance with the preferred embodiment of the present invention.

FIG. 5 is a flowchart illustrating the Energy Processes in accordance with the preferred embodiment of the present invention.

FIG. 6 is a flowchart illustrating the ULTRAGRID™ Process and integration with the Aquaponics System in accordance with the preferred embodiment of the present invention.

FIG. 7 is a flowchart illustrating the Master Control Process in accordance with the preferred embodiment of the present invention.

FIG. 8 is a diagram of the Nitrification Entity in accordance with the preferred embodiment of the present invention.

FIG. 9 is a schematic block diagram of an alternative embodiment of the Aquaponics System of the present invention illustrating the primary facility of the invention and highlighting particular interconnected elements thereof.

FIG. 10 is a flowchart illustrating the multi-level Aeroponics Process in accordance with an alternative embodiment of the present invention.

FIG. 11 is a diagram of the Bioreactor Enclosures in accordance with the preferred embodiment of the present invention.

FIG. 12 is a diagram of the Bioreactor Enclosures in accordance with an alternative embodiment of the present invention.

FIG. 13 is a schematic block diagram illustrating the Illumination Array in accordance with the preferred embodiment of the present invention.

FIG. 14 is a flowchart of the preferred embodiment of a method for operating the water supply of the present invention.

FIG. 15 is a flowchart of the preferred embodiment of the present invention of a method for manipulation of plant metabolism using spectral output.

FIG. 16 is a flowchart of a first alternative embodiment of the present invention of a method for manipulation of plant metabolism using spectral output.

FIG. 17 is a flowchart of a second alternative embodiment of the present invention of a method for manipulation of plant metabolism using spectral output.

FIG. 18 is a flowchart of a third alternative embodiment of the present invention of a method for manipulation of plan metabolism using spectral output.

### DETAILED DESCRIPTION

The following included description makes references to the accompanying drawings, which are provided for illustration of the preferred embodiment. However, such embodiment does not represent the full scope of the invention. The subject matter which the inventor does regard his invention is particularly pointed out and distinctly claimed in the claims of this specification.

The present invention advantage over prior art allows the important ability to produce food close to where it is consumed to reduce transportation and product losses and associated costs and ‘greenhouse gas’ emissions due to consumption of fossil fuels used in transport. Most of the world's population lives in and around urban areas it is essential for food production systems to be viable on land close to where the majority of the population lives. The current invention has the benefit of combining vertical stacking, ability to use non-toxic pest management, allowing the system to be cost-effective, minimal or no effluents, socially acceptable, and ecologically sustainable in urban and other niches not previously suitable for aquaponics 100, microalgae and organism production.

The present invention additionally consists of integration with product packaging 504 and onsite convenience food processing 502, dry storage 264 and cold storage 262 facility. The demand for local healthy product and convenience food, or tertiary processed food, is commercially prepared food designed for ease of consumption. Although restaurant prepared meals meet this definition, the term is seldom applied to them. Convenience foods include prepared foods such as read-to-eat foods such as packaged salads, frozen foods for example Garlic Salmon or Cajun Catfish TV dinners, shelf-stable products and prepared mixes such as the many various dehydrated products and mixes.

This invention relates to an aquaponics growing unit for growing plants and aquatic species in an enclosed and controlled space. Generally, standard soil based farming practice is dictated by such things as incompatible soils, diurnal seasonal changes, available solar hours for photosynthesis and climate conditions or a combinations of factors which can prove insufficient or unsuitable environments to support plant growth. This invention avoids soil contact related contaminants such as the bacterium E. coli is removed from occurrence by the preferred inventions lack of soil use and preferred methods eliminating external airborne atmosphere contamination exposure, potential soil and dirt contact. This invention eliminates the general or explicit need for application of fertilizers, herbicides and pesticides which cases environmental problems and health concerns and for humans themselves as consumers of the produce. This invention elimination of weeds, other unwanted plants and GMO genetically modified plant contamination that can reduce the value of completely ruin a crop and removing the plants from the risk from animals, insects and host of other pests including bacteria, pathogens and viruses.

Aquaponics 100 is a symbiosis fusion of an aquaculture system 106 with an aeroponics system 104 within a single symbiotic controlled environment. Water is circulated, or recycled, between the aquaculture system 106 and the aeroponics greenhouse 104. Aquaculture effluents such as aquaculture waste (which is rich in plant nutrients such as nitrogen and ammonia, but would be toxic to the aquaculture if not extracted in the aquaculture tanks) is transferred in the waste water out of the aquaculture tanks of the general aquaculture system 106 and into a microalgae bioreactor 130 and organism reactor production system 132 with which in turn may be fed into a filter feeder type of aquaculture 136 and then back to the aeroponics greenhouse 104 where the plants grow pursuant to their intake of the nutrient enriched aquaculture effluent and supplemental nutrient loading. Due to a microalgae bioreactor 130 and organism reactor production system 132 in conjunction of aeroponics has an intake of nutrient rich effluent from the water, which would be normally be toxic to the aquaculture unaltered, digestion handled waste cycle separating solid waste, liquid fertilizer and recycled water which is then transformed via biofiltration and filter feeders 136 is now potable and can then be transferred back to the aquaculture tanks for the ecosystem cycle to begin anew.

While each of the advantages offered by the present invention on their own provide significant advances over the traditional methods and systems, when considered together as a sustainable ecosystem that offers and addresses to fulfill the spirit of the invention as a complete solution with even greater advantages free of contamination, solutions that extend over an entire range of individual global problems comprising food and biomaterial availability, quality, quantity and toxicity.

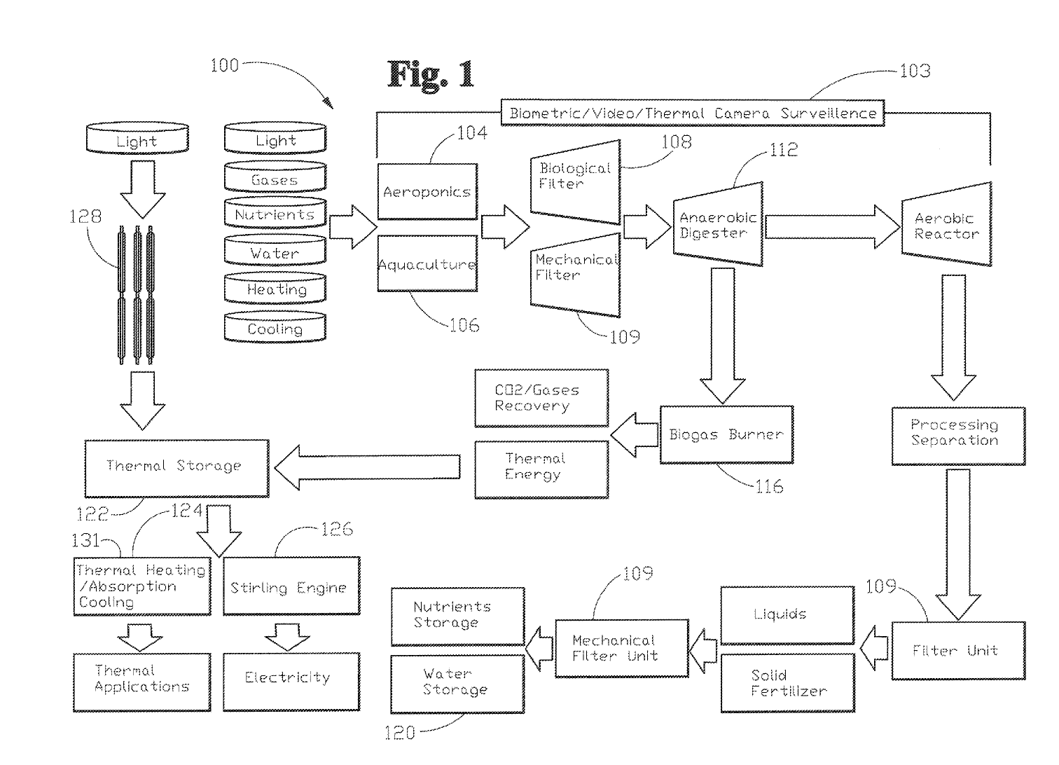

Referring to FIG. 1, a schematic block diagram of a system 100 of the present invention is shown. According to a first aspect of the present invention there is provided an aquaponics system 100 which includes the following: (a) a tank for housing at least one aquatic animal species 106; (b) a plant growing apparatus 104 for housing one or more plant species growing in an aqueous environment; and (c) a biofilter module 108 for receiving a waste stream comprising solid waste and water from the aquatic animal tank 106. The biofilter module 108 further comprises; a solids removal means 109; and biological waste digestion unit 112 for digesting solids from the solids removal means 109 to produce plant nutrients, which biological waste digestion unit 112 comprises a biological species that at least partially digests solid waste from said solids removal means 109 to plant nutrients. Whereby, in use, said plant nutrients are transferred to the plant growing apparatus 104 and at least a portion of the water is returned to the tank of the aquaculture unit 106. Further, it should be understood that inputs for the above described system as well as its various sub-systems comprise of at least one of the following: light, gases, nutrients, water, heating and cooling.

According to the present invention there is a provided plant growing unit comprising a germination chamber 404, grow out chamber 408, within which is provided a plurality of plant holding means supporting a plurality of plants and supplying a fluid nutrient mix to such plants; said growing chamber being provided with using adaptive biometrics, thermal imaging sensory and additional sensors means, illuminations means, temperature control means, and means for supplying said fluid nutrient mix to said plurality of plant holding means. In a preferred embodiment said chamber comprises an enclosed area enabling environmental inputs controls such as temperature, humidity, pressure, O2, CO2 and other gas mixture controls.

Prior art methods can be enhanced using the present invention's system and method of sustainable aquaponics 100 that vertically integrates unique aquaponics system 100 designs with alternative aquaculture feed sources, hatchery, fingerling production methods, alternative aquaculture/farmed fish/algae/organism grow out models 204, and renewable green energy sources that yields sustainable naturally organic produce output.

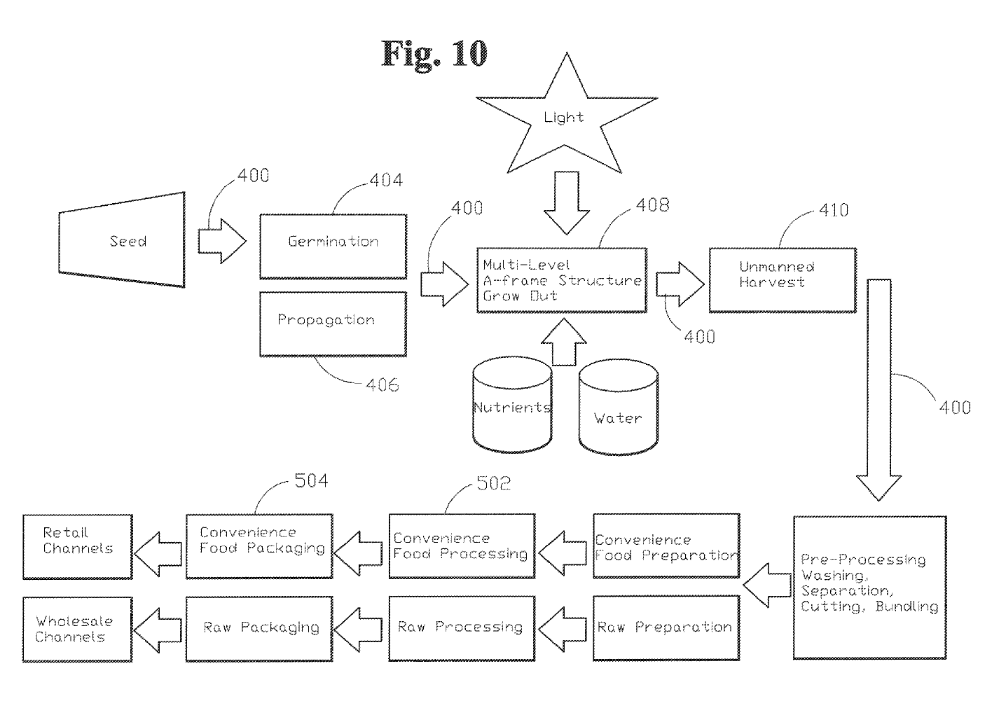

Present invention, as illustrate in FIG. 10 discloses a multi-level aeroponics system 104, which has further advantages over prior art that may be realized through the use of vertical stacking, efficient yield density and subsequent highly reduced footprint requirements. This can be an advantage in any situation where land for food production is expensive or there are other reasons for minimizing the footprint. Furthermore, the current invention may also aid in the reduction of problems associated with the high level of effluents in such a region due to its closed nature.

The present invention has addressed the above identified limitations by providing the waste treatment components of the system independent of the aeroponics 104 and aquaponics 100 growing areas. This has allowed the use of very little, or no, plant media within the plant beds. Surprisingly, this has allowed the applicant to provided a system which may provide higher production per square meter than existing traditional aquaponics systems 100 in the virtually complete absence of waste effluents.

Similarly on land that has not only high monetary value, but cultural, recreational, biological value etc. the current system is practical. Present invention would be attractive for example in areas such as in environmentally sensitive areas, limited land availability areas etc. Further advantages of the present invention is the ability to produce the local food and biological Material needs in the minimum possible land area near population manufacturing centers.

Another advantage of vertical stacking, as described in FIG. 10, and subsequent reduced footprint is that facility infrastructure, operational costs, as well as land acquisition costs can be minimized. Additionally, the use of more compact facilities allows for more cost effective application of Disease and Pest Management System (DPMS).

### Bioreactor

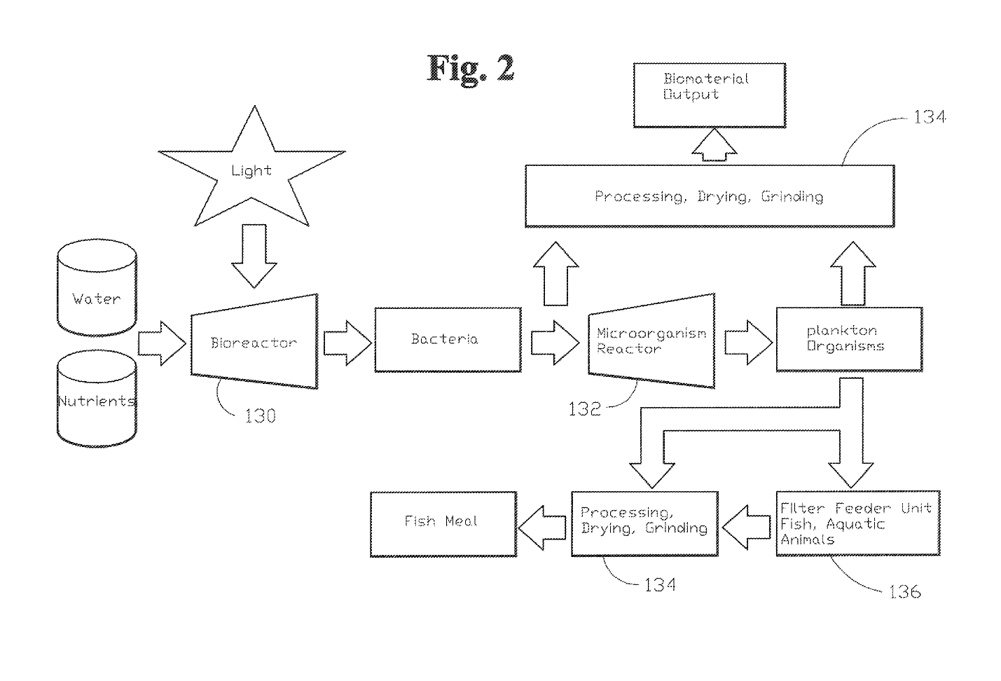

Referring now to FIG. 2, shown is a schematic block diagram of the bioreactor 130, reactor, filter feeder 136, fish meal, and biomaterial process in accordance with the system 100 of the present invention. The bioreactor system 130 of the present invention is an enclosed cycle bioreactor system 130 for culturing aquatic organisms. The bioreactor 130 uses adaptive biometrics, thermal imaging sensory and additional sensors for detection of product contaminations, product quality assurance tracking 616 of all methods, applications and product. Thus products can be quickly and easily identified and analyzed to provide additional information for the control system for enhanced production and high yields of microalgae and microorganism health. In particular it relates to a bioreactor system 130 that is a partitioned from the enclosed aquaculture system 106.

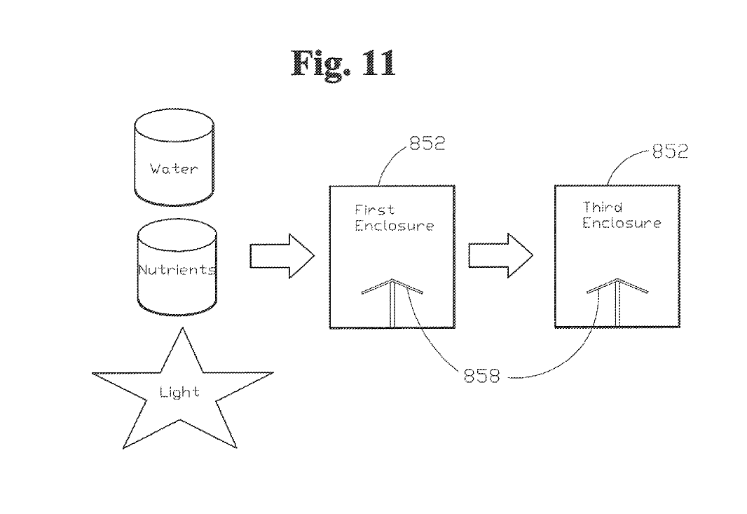

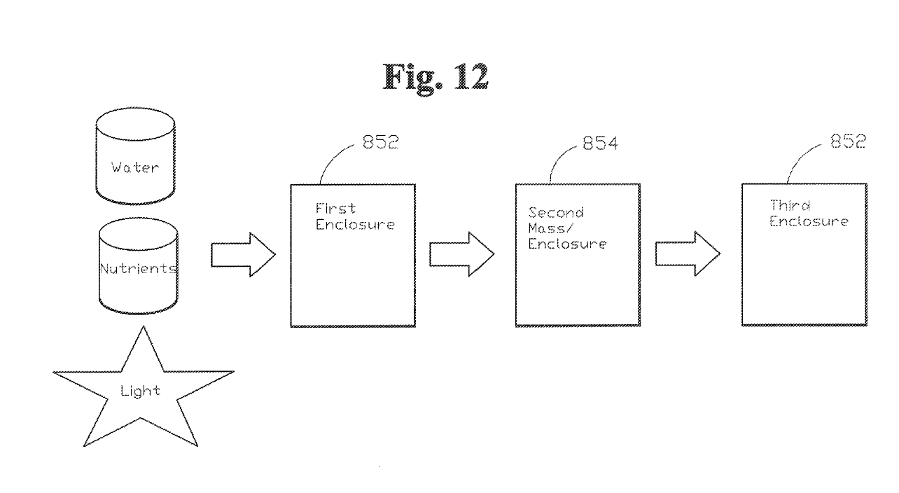

As illustrated in FIGS. 11 and 12, the present invention relates to a bioreactor system 130 for the cultivation of aquatic organisms using enriched water, wherein the system comprises separate enclosures for the enrichment of the water, utilizing one or more enrichment enclosures 852 and/or water masses 854, and the cultivation of the organisms. The advantage of the novel microalgae bioreactor 130 and microorganism reactor system 132 described herein is that it provides a solution to the supply problem of many natural and organic aquaculture products 106 by enabling a controlled closed cycle production of biomass of the organisms that produce these compounds using active interfacing of adaptive biometrics, thermal imaging sensory and additional sensors for detection product contaminations, product quality assurance tracking 616 of all methods, applications and product, can be quickly and easily be identified and analyzed to provide additional information for the control system. This is achieved by applying a novel feeding strategy that is specifically suitable for filter feeders. In this context, the term “filter feeders” refers to organisms that selectively feed upon suspended small organic particles such as microalgae, bacteria, detritus (dead particulate organic matter) and on dissolved organic compounds. The microalgae bioreactor 130 and microorganism reactor system 132 is able to supply a mixture of these organic nutrient components in adequate amounts to the organisms in culture.

In a preferred embodiment the filter feeders are aquatic animals, preferably invertebrate aquatic species. Food production and cultivation of the species is done in separate and/or isolated enclosures with self-contained environments. This makes these microalgae bioreactor 130 and microorganism reactor systems 132 different from prior art systems, such as Greenwater systems, in which enrichment of the water takes place within the enclosure with the cultured animals. Separated food production enables two features that are needed to establish an efficient filter feeder culture while enhancing isolated environmental cycles to avoid or at the very least minimize contamination or cross contaminations exposure. The first feature enables control of the concentration of suspended nutrients in the enclosure with the target species. Filter feeding species, only function well when constrained within a rather narrow range of food concentrations in the water fed to the targeted species.

Nutrients levels must be held high enough to sustain the nutritional demands of the targeted species (lower minimum threshold value). However, when the nutrient levels become too high (upper maximum threshold value), filter feeders naturally reduce their feeding activities. This reduced feeding activity will lead to a further increase of the suspended nutrient level, thus further reducing the feeding activity, so that the system becomes out of sync with an unhealthy environmental balance. The systems described according to the invention can be controlled in such a way that the suspended nutrient level will never exceed the upper threshold value. This can be accomplished using active interfacing of adaptive biometrics, thermal imaging sensory and additional sensors for detection product contaminations, product quality assurance tracking 616 of all methods, applications and product, can be quickly and easily be identified and analyzed to provide additional information for the control system Additionally, the preferred method of the present invention using adaptive biometrics, thermal imaging sensory and additional sensors for detection product contaminations, product quality assurance tracking 616 of all methods, applications and product, can be quickly and easily be identified and proactively providing additional information for the control system. This is achieved by continuous active monitoring of the suspended nutrient concentration, for instance by online measurement of Total Organic Carbon (TOC), which can be done by spectrometry or by wet oxidation analysis. When the carbon concentration reaches the upper threshold value, the nutrient supply is discontinued by switching off the transfer pump that initiates the flow. When the lower threshold level is reached, the nutrient supply transfer can be resumed.

The second feature enables a high-density culture. With targeted cultures, a large ratio between species volume and water volume is preferred from an economic viability standpoint. The circumstances of a high load of species, the total filtering activity of the species is too high to allow growth of microorganisms in the water surrounding the species. Mixed culturing of microorganisms with filter feeding species can only be done while the ratio between species volume and water volume is low. High-density culture of filter feeders therefore can be best done in systems in which nutrient production is separated from the enclosure containing the targeted species.

Another embodiment of the invention advantage over prior art is that the enrichment used can be nonselective or selective on a case by case basis. By enriching a mixed population of microorganisms, a natural diet and optimized environmental characteristics for the cultured species is mimicked and brought up to the level to promote the most optimal growth of the target species using active interfacing of adaptive biometrics, thermal imaging sensory and additional sensors for detection product contaminations, product quality assurance tracking 616 of all methods, applications and product, can be quickly and easily be identified and analyzed to provide additional information for the control system.

The enclosures may be make of an material that is non-toxic to invertebrates. Suitable examples include but are not limited to glass, plexiglass, PVC, polypropylene, polyesters, stainless steel and other similar materials. Within the enclosure, nutrients for the cultured species may be produced by enrichment. Thereto, the enclosure inoculated with water that may contain a mixed population of micro, nano and picoplankton. The inoculum may be supplemented with defined cultures of either phototrophic or heterotrophic microorganisms. Alternatively, an enclosure is inoculated only with one or more defined cultures of either Phototrophic or heterotrophic microorganisms.

The term “parallel” herein means that all enclosures that are similarly connected to other enclosures. The term “enrichment” refers to the stimulation and optimized environmental characteristics for the maximized growth of plankton. This may suitably be done by stimulating the growth of phototrophic plankton, or by stimulating the growth of heterotrophic plankton or by stimulating both phototrophic and heterotrophic growth simultaneously.

Extreme importance for overall health and control of plankton growth cycles is highly dependent on temperature based environmental control means. Said means may comprise a refrigeration unit and/or cold storage system 127 and/or heat pump and/or thermal heat storage unit and/or heating system provided to alter environmental conditions of the enclosed area. The refrigeration unit or heat pump of the unit may be powered by a thermal solar, photovoltaic solar system and/or energy generational input. Preferably said chamber includes climate control means. Preferably said adaptive climate control means is adapted to control one of more of the temperature, humidity and gas composition elements with the aquaponics system 100.

More than one enclosure can be run in parallel using different strategies for inoculation and enrichment in each enclosure. This method has several advantages. For instance, the heterogeneity of the diet may be optimized. Additionally, extra enclosures serve as a backup in case that one of the enclosures is not functioning properly and has to be restarted or becomes contaminated and needs to be cleansed and reinitialized.

Phototrophic plankton is stimulated by adding concentrated solutions of medium components into an enclosure and providing light to the enclosure to initiate organism growth. a person skilled in the art will understand that, in this case, in closed systems, medium components that are not toxic to the targeted species when supplied in the concentrations levels needed for maximize microorganisms growth yields, such as silicate and iron, can be added with the inflowing stream transfers between enclosures. Heterotrophic plankton may be stimulated through the addition of organic carbon source which will promote growth of heterotrophs, this species is non-toxic to invertebrates, amalgamated with other medium components that are required for heterotrophic growth. Suitable examples include a symbiotic relationship of glucose and glutamine, yeast extract and aquaculture species extracts. Within the enclosure is a mixing device, for instance airlift mixing, magnetic or mechanical stirring, in order to keep the nutrient particles in suspension.

One storage enclosure is selected to be used to maintain the cultured animals. Any type of tank may be used for this purpose, for example a raceway tank, a stirred tank, or an airlift reactor. The preferred enclosure preferably has optimized for environmental characteristics for maximized yield production and health using active interfacing of adaptive biometrics, thermal imaging sensory and additional sensors for detection product contaminations, product quality assurance tracking 616 of all methods, applications and product, can be quickly and easily be identified and analyzed to provide additional information of the control system.

The storage enclosure must be equipped with a water quality control system 601 in order to control the pH, the salinity and the levels of contamination such as nitrate and organic waste products in the water. It is essential hereby to apply a system that is not using mechanical filtration (i.e. no external loops with filters that remove particulate organic matter from the water should be applied), because such systems will rapidly remove a substantial part of the food for filter feeding organisms (particulate organic matter) from the water. Naturally produced organic matter, such as the material produced during operations usually consists of a rapidly degradable fraction, which can be consumed by aerobic, heterotrophic microorganisms within mere few days, and a resistant fraction that can be retained in aerobic waters for months and possibly years.

Organic matter sometimes forms larger aggregates, which are not suitable as food for filter feeders and which can clog parts of the system. To remove these particles, the bioreactor system 130 according to the invention may also comprise one or more devices to reduce the size of aggregates that are formed in any one of the enclosures. In another embodiment, size reduction is achieved by using filters 109, preferably stirred filters 858. In the case of closed systems, the filters are implemented in the connections between enclosures pens.

In another aspect, the present invention relates to a method for the cultivation of aquatic organisms comprising cultivating the organisms in a bioreactor system 130 according to the invention. Preferably, the aquatic organisms are filter feedings organisms, especially filter feeding species. Most preferred are invertebrate filter feeding animals, in particular the any aquatic species, which include animals such as bryozoans, bivalves, ascidians and filter feeding crustaceans, such as barnacles. In one embodiment, the invention is used for the cultivation of mollusks, clams and oysters but should not be seen as limiting to these species. Preferably, it is used for the cultivation of clams. In one embodiment, the system is used for the cultivation of the aquaculture in a closed bioreactor system 130.

During this period, part of the total amount of the cultured organisms may be harvested, for example for investigation or extraction of natural product. The bioreactor system 130 according to the invention allows for enhances animal growth compared to existing systems. In one optimized environmental embodiment, animals grew at least about 5% per week (increase in mollusks biomass). Preferably, animal growth is enhanced at least about 5%, 10% or 15% per week.

A person skilled in the art will understand that the system 100 of the present invention may also be used to product products which are normally not produced by the aquatic organisms, but which the organisms product upon genetic modification. The metabolite may be used in industry, preferably in the food, feed, paper and pulp or textile and/or bulk chemical industry, but more preferably in the pharmaceutical industry. The aquatic organism is preferably a filter feeding organism, more preferably a filter feeding animal, most preferably to mollusks and clams, in particular a tiger mollusks, such as the ones mentioned before. In yet another aspect, the present invention relates to the use of aged organic material to feed filter feeding aquatic organisms and species.

Setup and Operation of the System

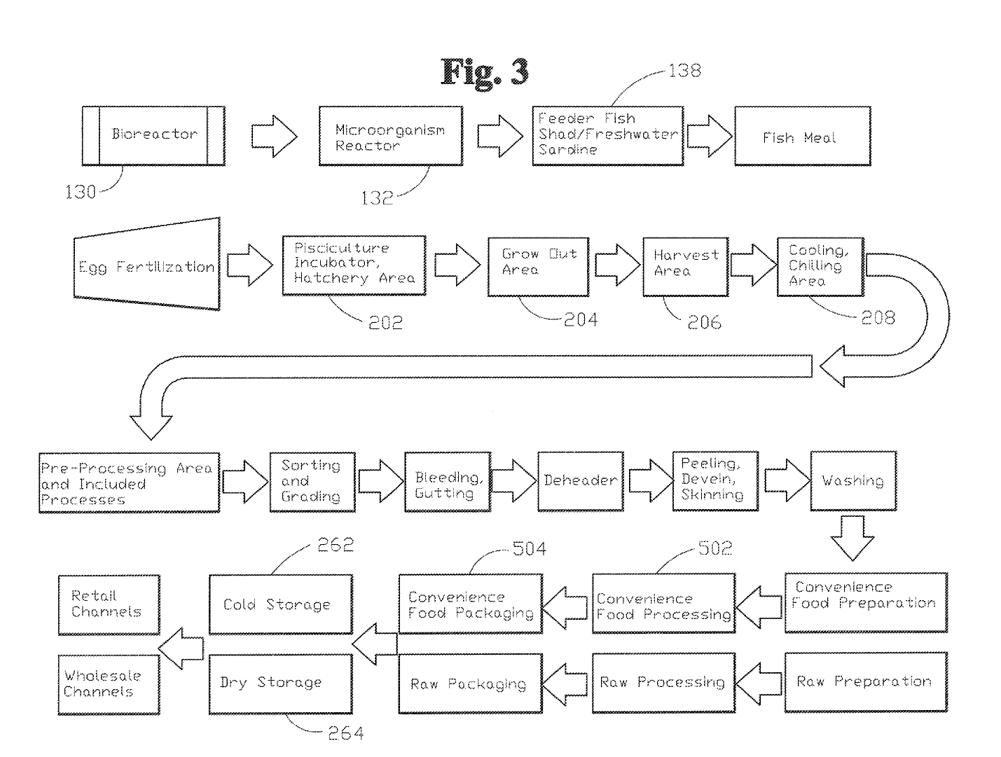

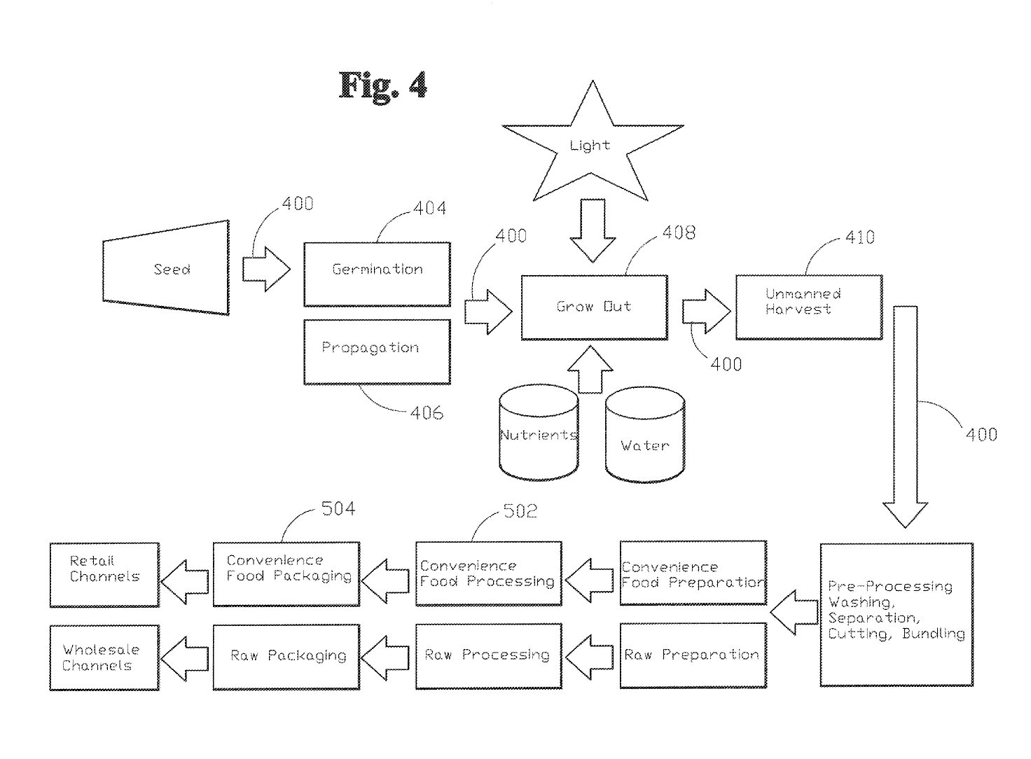

Following the outline, description and sample layout may be used to construct an aquaculture facility 106, as depicted in FIG. 3 in accordance with the present invention, preferably in combination with the aeroponics facility 104 as an outline diagrammatically described in FIG. 4, and description of facility start-up and operation of the integrated systems. Typically it's not critical that either the aquaculture 106 or the aeroponics 104 be the first to be constructed or to be set up first.

Referring specifically to FIG. 3, the aquaculture facility 106 of the system 100 contains the species of the aquaculture to be grown and can be separated per the selected tank. As indicated previously, virtually any species of aquatic life may be grown in any particular aquaculture tank. Prior to introducing any aquaculture species into any of the aquaculture tanks, a tank must be selected and prepped for use. Verification that the tank is clean with no harmful contaminants present and with such preparation as bacterium testing, checking control valves and input connections and tank test for leaks. With checklist items verified that selected tank is then filled to the proper level and when complete the last stage of testing commences for the selected species pH and temperature requirements, upon successful completion and verification the next steps are executed.

Next, air bubbler supply system input is activated to begin aeration and agitation in the selected tank. The air input valve is opened and the flow rates to the tank's airlines bubblers in the aquaculture tank are adjusted for the appropriate species requirement. The flow rate to each of the airlines bubblers in the aquaculture tank, if constructed as described herein, preferably will be set within safe margins for species needs. This may require active monitoring and analysis of the species in the selected tank and occasional adjustment to meet proper health requirements in relationship to maintain optimum fulfillment of individual species needs.

Prior to introducing the aquaculture species into the aquaculture tank, air and/or oxygen supply system is activated to begin aeration and agitation inside the enclosed mixing system in line with the water input circulation system, enriched dissolved oxygen in water should be kept between a minimum of 80 percent and maximum of 110 percent gas concentrations levels, which then introduces the water that should be at the predetermined temperature and pH balance needed for the specific species to the selected tank being readied for use.

Thereafter, the biofilters 108 that are used to filter the water coming from each aquaculture tank and holding tank are populated with microorganisms capable of converting ammonia to nitrite and nitrite to nitrate. This will occur naturally, where pond water is used, but it may require as long as several weeks for a sufficient population to accumulate. Prepared cultures of microorganisms for seeding the biofilters 108 are commercially available and will significantly reduce the time required to adequately populate the biofilters 108, and possible require only a few days. The biofilters 108 are adequately populated when the ammonia concentration decreases and the nitrate level increases.

The air flow to the fish tank should be adjusted so that a sufficient upward lift is generated to produce a current on each side of the tank. This current preferably extends substantially across the width and depth of each section of the tank. most preferably, the current will extend from top to bottom and across the entire width of the tank. The flow should be sufficient to circulate the volume of water on each side of the tank about every two minutes. Thus, it will be understood that the width and depth of the tank, the dimensions and position of the column of air bubbles and the upward current produced thereby must be relatively sized to produce a rolling current of the desired dimensions.

The preferred embodiment utilizes the advantages of integrating an artificial intelligence and machine learning control system 624 with active interfacing of adaptive biometrics, thermal imaging sensory and additional sensors for detection product contaminations, product quality assurance tracking 616 of all methods, applications and furthermore, products can be quickly and easily identified and analyzed to provide additional information for the control system for active control.

A common air source simultaneously serves three important functions. The air released at the bottom of the tank and rising up through the channeled bubblers provides an ample source of needed oxygen to the aquaculture held in each tank. this rising air column sets in motion a continuous churning or rolling current which agitates the water throughout the tank. The air bubbles carried with the current are dispersed throughout the tank water thus maintaining a high dissolved oxygen content in the water throughout the tank.

Aeration and agitation of the holding tank may be adjusted as in the fish tank assembly. Because the preferred holding tank is much smaller than the aquaculture tank and has only a one-side arrangement, less air will be required to generate a sufficiently sized current. an acceptable flow rate for the holding tank described herein is about 40 CFM, but this may vary according to the dimensions of the tank.

Having activated the aeration and agitation system and prepared the dissolved oxygen of the tank, the aquaculture tank now is ready to receive specimens. Preferably, a population of fingerlings and/or hatchlings is introduced into the smallest transfer compartments. The maximum number of aquaculture species that can be put in a compartment will vary according to the size of the compartment or, more specifically, the volume of water in the compartment. This can be determined by known principles. Generally, about 3 to about 10 pounds of aquaculture species per cubic foot of water will be appropriate, each species has its specific needs and requirements.

An optimum growth period, which is the optimum length of time a population of aquaculture species should remain in a compartment, should be determined for the selected species using monitoring provided by inclusion of an artificial intelligence and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system. This is based on known principles and will vary with the species of aquaculture selected. For example, 28 days is a suitable growth period for channel catfish.

The present invention also preferably comprises an oxygen system generally consisting of a pressure swing absorption system as a mixture system to supplement a common atmospheric air supply system. It will be understood that pure oxygen is a non-toxic gas comprising a pure or nearly pure concentration of oxygen could be used instead of air. However, atmospheric air is the most abundant and least expensive source of oxygen presently available, and as such, is the preferred primary oxygen source. As used herein, “air” or “air supply” refers to any oxygen gas mixture containing sufficient directly usable amounts of oxygen.

After the first growth period, the divider is removed and the batch of fish is herded into the next compartment. After the dividers are replaced, a new batch of fingerlings and/or hatchlings is introduced into the smallest transfer compartment. In like manner, at the end of each growth period, the batch of fish in each compartment is advanced into the next largest compartment and a new batch of fingerlingers and/or hatchlings is introduced into the first transfer compartment. The aquaculture in the aquaculture tank preferably is fed a prepared diet. The diet should be given according to the selected species requirements.

As further outlined in FIG. 3, in each grow out tank 204, a batch of fully grown aquaculture will be ready for harvesting 206 from the tank in relationship to size and weight required is reached. At this time, the aquaculture batch to be harvested is transferred to the holding tank while awaiting final processing. The harvested aquaculture 206 are maintained without feeding in the holding tank until their alimentary systems are emptied or purged in relationship to the species being processed. This eliminates undesirable flavor and odor from the aquaculture species. After this period, the aquaculture is ready for final processing, packaging, labeling for market or frozen for storage as outlined in FIG. 2. Thus, the total time in the aquaculture tank for each batch of aquaculture is dependent on the species and the requirements set before the species is ready to be harvested. For instance a fresh batch of fully grown catfish is potentially produced once a month in a production-line method and fashion.

The temperature of the aquaculture water should be maintained at an optimum level for the specific species of aquaculture involved. The temperature of the air in the aquaculture facility should be maintained at a selected level as this has a large effect on the water temperature. The optimum room temperature for the aquaculture facility 106 will be dependent on the species of aquaculture involved. The temperature of the air used to aerate the aquaculture water also has a direct effect on the water temperature. Accordingly, the temperature of the air supply to the airlines may be adjusted on an as needed basis. For example, when the air supply is too hot or too cold, input from cold or warm sources and/or storage may be used. The metabolic processes of the aquaculture give off a certain amount of heat as does the natural oxidation of the solid waste products produced by the aquaculture. Thus monitoring and analyzing temperatures in the air, water and airlines needs to be maintained for optimized environmental characteristics for proper species health.

As the population of aquaculture in the aquaculture tank is increased, monitoring water quality, temperature and dissolved oxygen likewise is also gradually systematically increased. Preferably, active monitoring and adjusted levels of optimized environmental characteristics for the selected species are maintained. For proper health at varying growth stages this can be achieved by inclusion of an artificial intelligence and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system.

The flow of incoming water to the aeroponics facility 104 should be regulated in such a way so that a unit of water is circulated through the system about every 48 hours. In a preferable embodiment, an adequate flow rate is set and adjusted in relationship of the facility and the selected aquaculture species. The flow rate in most instances will be regulated by adjusting the flow to match the selected species requirements. In the preferred embodiment, as previously described, the circulation rate is a function that is set when aquaculture species is selected. Thus, the air flow and water circulation valves in relationship to the selected aquaculture species is used to regulate the air flow and water circulation of aquaculture water as a basis of water input to the aeroponics facility 104. The composition of the circulating water should be monitored regularly. Adjustments should be made as necessary to ensure that the water contains the necessary nutrients for the selected aeroponics plant type and that the nitrogenous wastes is being maintained at or below a non-toxic level for the aquaculture at optimum levels for aquaculture health.

The water should be tested for plant-required nutrients, such as phosphorus, calcium, potassium and others. The water may be enriched or diluted as necessary, but only with substances that are compatible with the tolerances and requirements of the aquaculture, plants, feeder filter aquaculture and the organisms in the biofilter 108. Preferably, relatively constant levels of these substances will be maintained for optimized environmental characteristics to assure even and predictable plant health and growth. This can be achieved by integrating this with an artificial intelligence and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system.

In this regard it should be noted to retain organic labeling it may be required that there would be no pesticides of any kind used on the plants in the aeroponics system 104. These substances would likely be transmitted by the roots into the effluent water and could prove toxic and potentially fatal to the aquaculture and other useful bacterium in the system. Thus, the plants grown in accordance with the present invention are completely organic, pesticide and contamination free.

The ammonia concentration levels should be monitored constantly. It should be maintained at levels specified according to species and system needs. The nitrate and nitrite concentrations levels should also be monitored constantly to maintain proper balance within the system. The concentration of dissolved oxygen in the aquaculture water should be tested constantly, preferably at minute intervals. Concentration levels may be maintained by enriching the air supply with pure oxygen to increase the concentration of oxygen in the air supplied to the aquaculture tank airline supply in relationship to aquaculture species requirements. The pH of the fish water should be monitored constantly and adjusted if necessary to maintain a healthy pH balance in relationship to aquaculture species requirements.

The solid aquaculture waste may accumulate in sediment traps, pipes and beds of the aquaculture tanks. This may be cleaned and/or removed from the system on an as needed basis or periodically. The total volume of water in the system should be monitored, preferably by observing the water levels in the aquaculture facility 106. However, it will be appreciated that, because of the symbiotic relationship between the aeroponics network 104 and the aquaculture network 100, the system operates on a substantially constant body of water from one facility to the other. There is no need to replace portions of the aquaculture water in order to maintain nitrogenous wastes at nontoxic levels. When the described systems are operating efficiently, the only additional water required will be the relatively small amount needed to replace water lost by evaporation and plant transpiration.

As depicted in FIG. 3, the aquaculture farm 106 of the present invention is housed in an enclosure, such as the aquaculture facility 106. A prebuilt portable building or prefabricated structure may suit this purpose. Preferably, the fabricated enclosure is adapted or may be adapted for excluding sunlight and maintaining a relatively constant environment such as temperature, humidity, gas management for the aquaculture facility 106. Preferably the enclosure would consist of an insulated, leak proof, moisture proof, pressure sealed and rust resistant enclosure to maintain environmental. While sunlight preferably is totally excluded, a general lighting system may be installed for human access usage.

Referring back FIG. 1, general building features of common art or of a conventional nature are not deposed or are depicted in the drawings. For convenience, several doors and/or access and/or passageways may be included. For loading and unloading, a single or a plurality of loading docks(s) is generally preferable, although a single or a plurality of large overhead door(s) may suffice. These doors are of conventional construction and would preferably use insulation and are indicated only diagrammatically. The aquaculture facility 106 preferably includes an area and/or enclosed area for housing the breeding and/or hatchery area, may also include a work area. The aquaculture system 106 may consist of multiple floors and/or levels to increase the density and yields of species available for processing 134 in each aquaculture facility 106.

Referring back FIG. 2, ideally, the aquaculture facility 106 includes a modular bioreactor 130 with a module for production of Phytoplankton and may include a reactor module for production of Zooplankton and may have additional modules for other bacterium growth as needed or required. The aquaculture facility 106 preferably includes an artificial intelligence and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system.

In accordance with the methods of the present invention, aquaculture species may be grown by maintaining them, as described, in the above aquaculture tanks. The water from the aquaculture tanks are circulated through the aeroponics facility 104 and back to the aquaculture facility 106. Preferably, the water in the aquaculture facility 106 is continuously circulated through the filter feeder aquaculture 136 and then through the biofilter 108 and digester unit 112 which preferably is in an isolated area. Similarly, plants in an aeroponics facility 104 may be grown by a method which includes circulating a substantially constant body of water from the aeroponics facility 104 to the aquaculture facility 106, as described.

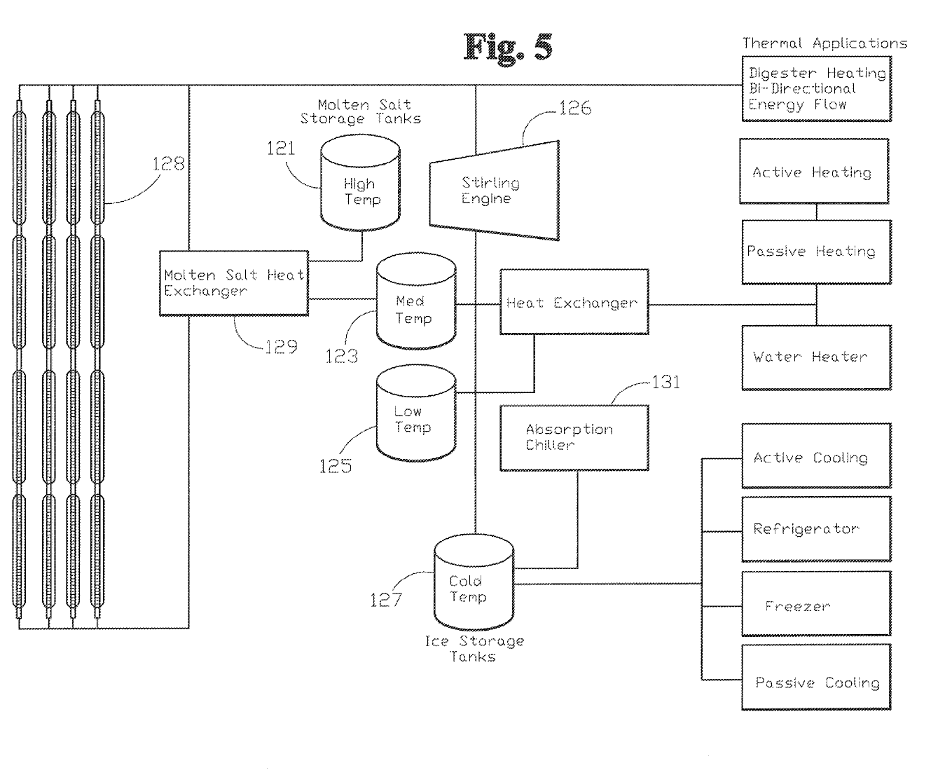

Referring now to FIG. 5, a flowchart illustrating the energy processes in accordance with the present invention is shown. The system 100 includes a primary core generation element that is comprised of the solar thermal component. Potential additional integration includes hybrid wind and/or photovoltaic solar power energy generating devices. Supplemental thermal energy will be achieved from digester 112 provided bios gas burner thermal generation. These devices form the quintessential embodiment, integral for establishment of the distributed energy generation source provisioning for (a) electrical, chemical and grid connection, (b) bidirectional networked data communication, and (c) control for interconnection and interoperability.

The present invention forms a system defined by a set of integrated processes for the production and storage of electrical, chemical and thermal energy. Production and processing of thermal energy is typically for the purpose of thermal energy vessel storage and geothermal storage for later use. Other objects, features, and advantages of the present invention will be readily appreciated from the following descriptions and listed improvements.

The preferred embodiment of the present invention also consists of a core renewable energy storage 122 with a common shared intelligent interactive energy generation system and intelligent machine learning system. The core energy generating device envisioned that shall hereby referred and designated as a Solar Thermal Array Conversion System (STACS) This is effectuated by fully accommodating and promoting the usage of all available usable thermal energy collection be thermally communicated into heat energy storage 122 and/or transference to cold energy storage 127.

Another improvement is using commercial grid scale electrical energy surplus via electrical derived thermal generation for commercial grid scale thermal storage. This enables storage in the hundreds and potentially thousands of kilowatt hours, expandable to megawatts hours of term storage, energy available on demand. Another improvement is with ability to store excess wind and/or photovoltaic solar electrical energy as commercial grid scale thermal energy storage 122, the excess electrical energy which is now stored as thermal energy can be used as an active or as on-demand energy source for energy generation for commercial grid base-load or can be used to meet high peak demand load needs for load stability and voltage stability and localized power quality commercial grid efficiency 706.

Further improvement of the present invention is the complete integration of localized onsite thermal and geothermal energy storage 122 for use as an on-demand energy source for energy generation for thermal storage 122 maintenance heat generation, grid base-load, intermediate base-load peaking support or can be used to meet high peak demand load needs for load stability, frequency matching and voltage stability and grid efficiency.

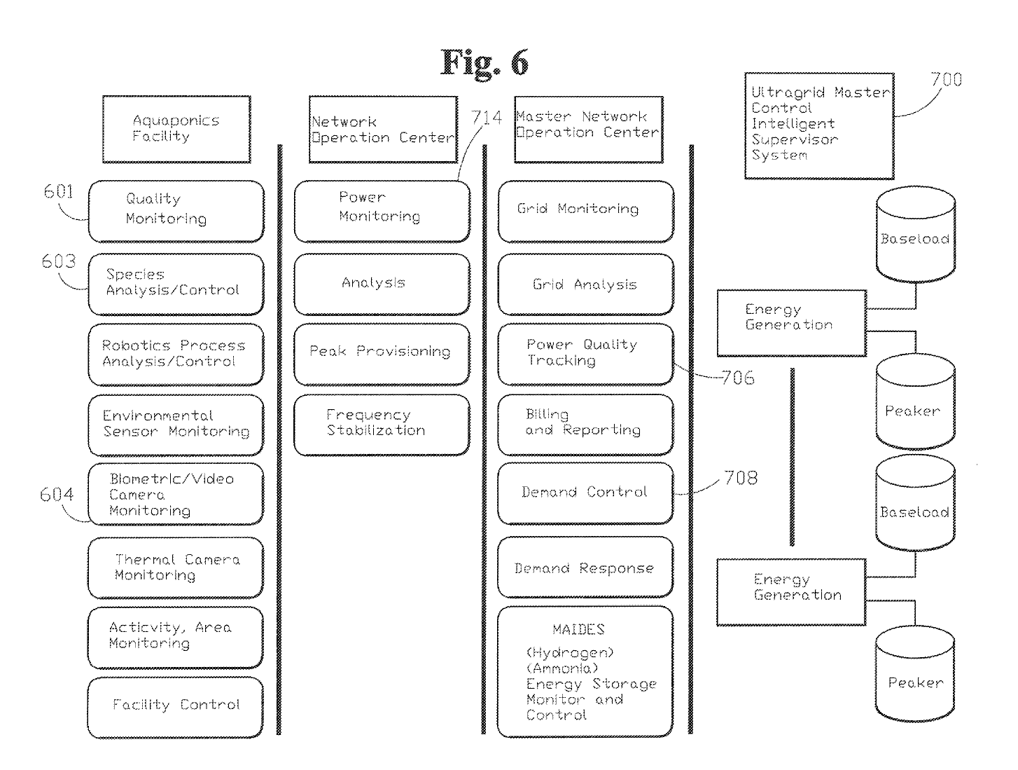

Looking to FIG. 6, depicted is the integration and inclusion of compatibility with the ULTRAGRID™ system 700, which comprises a complete line of consumer and commercial products and services for maximizing energy generation, storage and provisioning for end use. Enhanced efficiencies and energy stability through localized commercial grid provisioning systems are realized through a combined software and hardware solution. Additionally other device power and control systems can be substituted. ULTRAGRID 700 is designed in layers of components potentially consisting of energy generation, energy storage 122, energy provisioning, grid layer, consumer layer an end user component layer. Using layering will allow for simple logic integration, flexible information access, adaptability and expandability, rapid response, quick and easy installation and robust and secure operation. Another improvement is integration and compatibility with external software packages and other device power and control systems can be substituted.

Software for consumers allows for local and remote use to analyze and control personal energy use and enables integration in the ULTRAGRID™ ZHI home control and security system 700. Hardware for consumers comprises of standalone plugin adapters namely the ULTRAGRID ™ ZI 700 allows common household appliances to be plugged in allowing them to become smart appliances. Additionally other currently available control devices can be substituted for compatibility and continuity. ULTRAGRID™ ZAI 700 enabled smart devices such as televisions, refrigerators and like user owned appliances, utilizing a common data interface and network, are capable of monitoring and sharing user usage for machine learning applications.

Additionally the system will monitor STACS thermal storage grid connected sites for grid energy load balancing for nominal load provisioning to enable reserve capacity generation capability for power quality 706 and energy availability enhancement. Further improvement will allow loss of an energy generator's power to be reallocated and provisioned from localized storage to an online and available status, this assist mode from the local system and other active system nodes is initialized in response to ULTRAGRID™ 700 command and control activation to prevent localized grid collapse and power quality fluctuations 706. Another improvement is the inclusion of ULTRAGRID™ 700 compatibility allowing communication from all sites and manages their status from a primary centralized command-and-control integrated network operations center. Said operations center, through the interconnected networked data control systems and subsystems, allow directing energy where and when needed and offering beneficial recycling and reclamation of waste energy and heat. A further improvement is enhanced consumer power quality 706 and grid stabilization during diurnal cycle with its variation and seasonal balancing requirements. This is effectuated by using localized consumer distributed thermal storage during prime time energy usage which occurs naturally during daytime hours. This can be supplemented to maintain optimum availability and reliability via external grid energy to thermal conversation during off peak hours.

Another improvement is the reduction of complete elimination of overlapping and redundant subsystems, reducing part counts and excess energy usage from elimination of duplicated systems and subsystems. The prior art depended primarily on efforts in engineering device efficiency and decreasing manufacturing costs. These methods are helpful but are limited in their scope and effectiveness due to the incremental enhancement typical to this type of development. The prior art relied heavily on modest evolutionary adaptations versus much more in depth radical changes.

The present invention differs from other prior art from inclusion and incorporation of thermal solar, wind, photovoltaic solar with integration to thermal storage and geothermal storage components. The present invention differs from other prior art systems from the above integration through electrical generation, heat for thermal applications, cold for cold thermal applications, while removing redundant components and their processes; thereby reducing energy input requirements. Prior art uses additional energy input to remove the heat to cool the areas within a consumer's enclosed area thereby reducing energy usage efficiency wherein the present invention harnesses the heat energy for beneficial work.

The present invention users the waste heat generated from the electrical generation process for use as the input energy as heat source for ammonia cooling and vapor cooling processes, water purification, desalination and water heating application processes, thus creating additional benefit of using available expended energy versus prior art creating energy loss and inefficiency by its inferior design. The present invention using a common thermal and electrical grid to reduce losses from inefficient and unnecessary conversion and transference, thereby increasing efficiency and promoting reduced energy needed and materials required for cooling applications and processes. The system of the present invention is advantaged by combining the localized systems into an efficient primary commercial grid energy system versus prior art needing and using multiple electrical and thermal distribution systems and transformation connections and conversions. These additional transformation connections and conversions create additional energy loss and efficiency loss with each blind connection.

Preferred embodiment is accomplished by facilitating electrical, thermal as well as chemical interactions and energy conversions through interconnecting a hybrid wind and solar energy generation system. Alternatively geothermal, hydroelectric and other grid energy connected input sources may be substituted.

Primary embodiment efficiencies and cost effectiveness is made possible from its quintessential energy generation capability from the improved and inclusive hybrid energy generation system and paired with its waste heat recovery system using reclaimed energy to actualize and realize the maximum benefits of using all available system resources. maximizing infrastructure utilization to achieve lowest possible levelized cost of energy is achieved by monetizing capital intensive fixed assets while reducing overlap and needless, redundant processes. This produces substantially reduced investment capital requirements, encapsulated by enhancing greater return on invested capital expenditures.

The disclosed embodiments provide a system that also generates electricity and heat energy for the purpose and production of electricity and thermal application use within the system and outside the systems as well. During operation, the system uses the hybrid mix of wind and solar to maximize day and night time electrical and thermal energy generation. Connection is made to an intragrid for internal industrial usage or as an external grid energy supplier. Additionally geothermal and hydroelectric or external sources can be used for electrical energy generation input.

Concentrated thermal solar system is deployed to collect thermal energy to be transferred and then stored into a high temperature thermal storage system 121. During night time and inadequate thermal collection periods, system taps its reserve of heat and cold thermal storage for application usage or electrical energy generation. Alternatively geothermal and other electrical and chemical reaction for thermal generation may be used for thermal energy collection.

In some embodiments, selectively transferring the heat from the high-heat-capacity fluid to the working fluid involves disposing a thermally insulating component between the high-heat-capacity fluid and the working fluid to retain the heat in the high-heat-capacity fluid, and repositioning the thermally insulating component to transfer the heat from the high-heat-capacity fluid to the working fluid through a thermally conductive component.

The high temperature thermal storage system 121 is deployed for the primary purpose of providing on demand thermal energy, this thermal energy is needed for thermal application and thermal to electrical conversion application use. Additionally, the system deploys a cooling system and chiller system 208 to provide proper pressure and cooling for localized cold storage system 127 and for further cold temperature application requirements.

The high temperature thermal storage system 121 is also deployed for the secondary purpose of providing thermal energy needed for thermal exchange using a work fluid to cause a turbine's shaft to rotate to cause rotational work energy, stirling cycle applications 126, and/or gas/working fluid expansion and contraction to cause usable work. The working fluid can also cause gas and/or working fluid expansion and contraction applications to cause force on a piston to cause motion for the purpose of providing usable work. Some embodiments use rotating blades comprising of a least one of the propeller, an impeller, one or more paddles and a drum. Some embodiments use a working fluid that is associated with a low boiling point. Working fluids can then be reclaimed for energy recycling and processed for system reuse.

In some embodiments, the system also uses an insulated vessel or geothermal storage to retain the heat in the low-heat-capacity fluid. In some embodiments, the thermally conductive component is component having high thermal conductivity, such as a metal surface, a manifold, a conductive rod, and a radiator. Finally, the system uses the transferred rotational energy to generate work or torque.

Additionally, in some embodiments, the transferred high-heat-capacity fluid boils the low-heat-capacity working fluid. Rotational energy may then be generated by exposing a compressed gas and/or working fluid in a cylinder to expand the gas and/or working fluid to provide force to a piston which then exerts the movement to a rotation on a crankshaft or of linear movement of a linear generator. Using the rotational energy or linear movement to drive applications or components such as mechanical linkage, swash plate, compressor, pump or electric generator.

Generated rotational and/or linear work energy is utilized by transferring the shaft Rotation and/or linear movement to provide a water pump the energy needed for incoming water to become pressurized to force water through the water purification systems. Examples of the aforementioned water purification systems include desalination, distillation, and reverse osmosis. After purification, the product water can then be stored in tanks and/or elevated water tanks 120, as an additional energy storage method 122 for on demand use.

Next, generated rotational and/or linear work energy can be used to provide rotational and/or linear energy to drive compressors to establish adequate operating pressure. This in turn enables pressure swing absorption to function properly, this process allows separating, isolating and storing gases and/or working fluid as an additional energy storage method 122 for on demand use. Lastly, generated rotational and/or linear work energy can be used to provide rotational and/or linear energy to drive generators to provide electrical energy production. This energy can then be transferred into the internal grid network for system use for additional hydrogen production through powering electrolyzers or made available as a grid energy supplier.

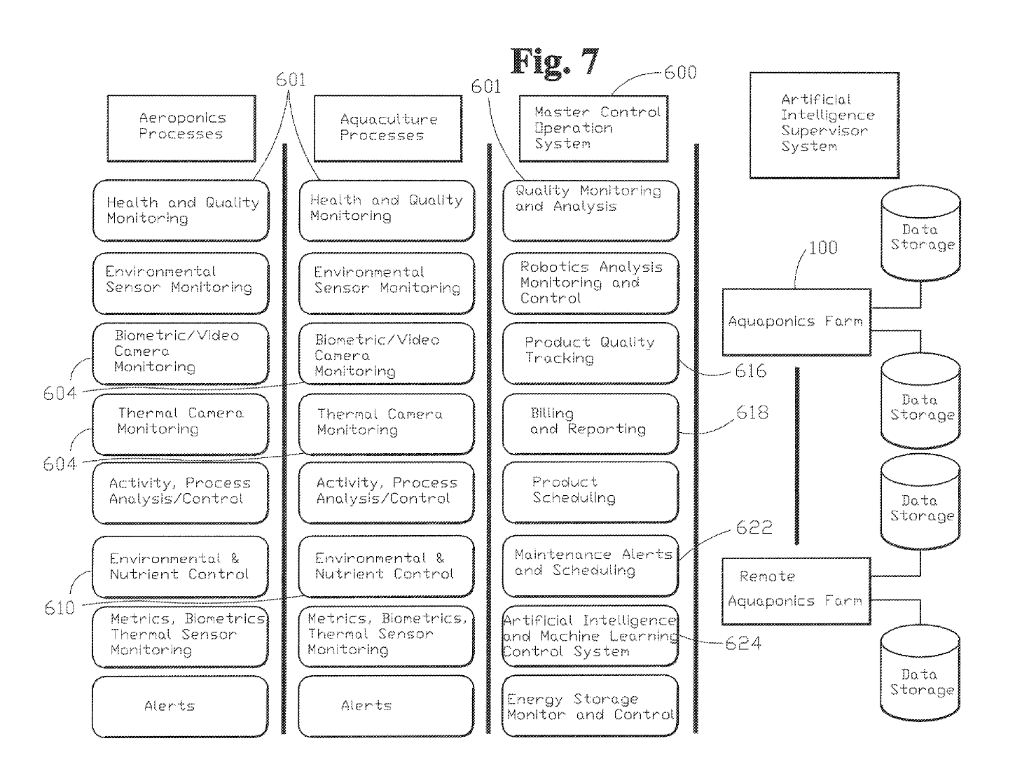

As illustrated in FIG. 7 the master control unit 600 and its sub-process integrate with the system 100 of the present invention. The invention can be generally characterized as a hybrid aquaponics system 100 for use with a plurality of grow out units 204 containing a fluid and/or fluid system, comprising a main control unit 600 for receiving feedback. Further novel methods would include using adaptive biometrics, thermal imaging sensory and additional sensors for detecting product contaminations, product quality assurance tracking 616 of all methods, applications and product. Said products can be quickly and easily identified and provide additional information for the control system wherein at least one pump control unit in electronic communication with the main control unit 600, and at least one gate control unit in electronic communication with the main control unit 600.

In another embodiment, the invention can be generally characterized as a method of using a microalgae bioreactor 130 and organism reactor system 132 for use with a plurality of grow out units 204 containing a fluid and/or fluid system. The method comprising: providing a microalgae bioreactor 130 and organism reactor system 132, the system providing and comprising providing a main control unit 600 for receiving feedback. Further novel methods would include suing adaptive biometrics, thermal imaging sensory and additional sensors for detecting product contaminations, product quality assurance tracking 616 of all methods, applications and product. Said products can be quickly and easily identified and provide additional information for the control system and providing control, providing at least one pump control unit in electronic communication with the main control unit 600, and providing at least one gate control unit in electronic communication with the main control unit powering the system and providing electronic communication with a master control system 600 running said system.

The preferred method of the present invention primary objective is to move beyond industry best practice and advance an energy-efficient aquaponics facility 100 that could be delivered and operated below the cost of a typical large aquaponics 100 or aquaculture facility 106 to set itself apart from past prior art as the most efficient aquaculture infrastructures 106 at the lowest possible costs. The design flow logic was to custom design, from the group up, the data center with its cooling and energy backup with built in energy storage 122, custom build its control system design, tank design interconnection, transfer system and building implementations to enable smooth integrations and transformations into a reliable and sustainable aquaculture system 106. The system of the present invention substantially differs from prior art. The present invention's use of renewable energy, preferably with thermal energy, along with its use and reuse of recovered and recycled waste energy for enhanced uses and purposes is a distinct advantage over prior art. This holds especially true with respect to the absorption cooling 124 integration that aids in providing cold storage 127, when necessary, as well as on demand energy to cool.

Referring back to FIG. 2, the present invention relates to a bioreactor system 130 for the cultivation of aquatic organisms using enriched water, wherein the system comprises separate enclosures for the enrichment of the water and food production on the one hand and the cultivation of the organisms on the other hand. Separated food production has several advantages. It enables four features that are needed to establish an efficient culture. In the first place, it enables control of the concentration of suspended food in the enclosure with the organisms. In the second place, it enables high-density culture. Additional advantage can be realized by this invention from the system 100 operated by a progressive artificial intelligence and machine learning based computer controlled system 624 that uses adaptive biometrics, thermal imaging sensory 103 and additional sensors to detect, analyze and control microalgae production in the bioreactor 130 amalgamated with the microorganism reactor 132 production growth cycles which includes adaptive biometric and thermal imaging analysis 604, monitoring and control for maximizing metabolism efficiency, reducing product loss, reducing colony deaths and deliver high production yields. In addition, a partitioned and/or isolated system allows pretreatment of the produced food before it is supplied to the organisms. Furthermore, the system according to the present invention allows that water from the enclosure containing the organisms may be used as medium for the food production enclosure. In this way, a significant reduction of microalgae and microorganism production loss and includes the reduction of the amount of water that is used may be achieved, and little or no water needs to be discharge. Additionally, water can be collected by climate control means and waste management system, this water may then be reused by the nutrient mix supply system.

The present invention has the following, but is not limited to an, on-site renewable energy generation and energy storage unit 122, as well as a production unit 104, an aquaculture production unit 106, a cold storage facility 262, a dry storage facility 264, a bio-filtration/feeder filter module 136, an aeration module, a bioreactor module 130, a digester module 112, a hatchery module, a germination module 202, culminating to product a high yield yet low-waste, self-sustaining food production system.

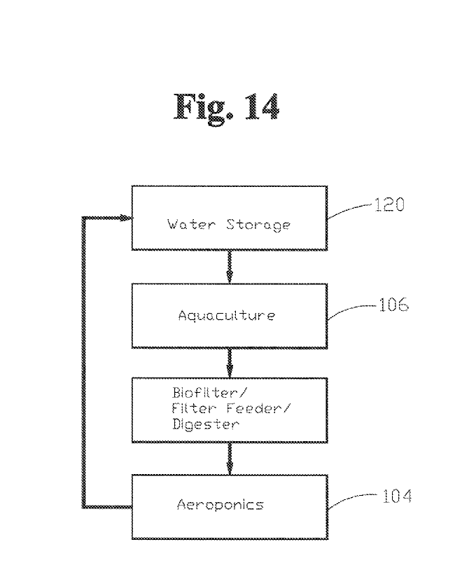

Referring now to FIG. 14, a flowchart of the preferred embodiment of the method for operating the water supply of the system 100 is shown. Aquaculture 106 generally has excretions that can accumulate as pollution in the water from the aquatic species being raised, increasing toxicity. In the aquaponics system 100 of the present invention, waste water from an aquaculture system 106 is then cleansed and purified by channeling the liquid through an aquaculture filter feeder 136 and biofilter component 108 (solid waste is transferred to digester unit 112 for recycling), then ducted to an aeroponics system 104 where the nutrient elements are naturally processed and effectuated by the plants as nutrients. The reclaimed clean water is the recalculated for use in the starting cycle in the aquaculture system 106.

Through its operation, the present invention forms a uniquely sustainable high yield complete ecosystem cycle providing a local flood production facility without prior arts requirements for external energy input form utilities or material input usage inefficiencies causing excessive material input requirements. By way of review and introduction, the present invention concerns an on-site system for the integrated production, processing and storage of ultra-high yield aeroponics 104 and aquaculture products 106. The system consists of a series of discrete and highly scalable production modules and cooperative systems with efficient recycling methods and processes to provide for the sustainable production of renewable energy generated organic consumable and commercial products.

Because the aquaculture facility 106 is enclosed, its environment can be monitored and artificially controlled and maintained, further novel method would include using adaptive biometrics, thermal imaging sensory and additional sensors for quality assurance tracking 616 of all methods, applications and product, can be quickly and easily be identified and providing additional information for the control system and use of infrared and ultraviolet and other light wave frequencies for thermal imaging detection and identification, kept contaminate, disease and pest free environment. In the absence of natural light and daily temperature fluctuations, the diurnal growth variations normal in outdoor aquaculture farms 106 are not present, and the aquaculture species will grow 24 hours a day, 365 days a year. Moreover, because seasonal variations in climate are eliminated, the unit can be operated successfully year round in any geographic area.

The preferred method of the present invention uses adaptive metrics, biometrics and thermal imaging sensory analysis including additional input sensors for analysis, monitoring, and control with integrated robotic automation 400 and maintained symbiotic artificial intelligence controlled system 624 providing a balanced environmental friendly based facility ecosystem.

The preferred method of the present invention advantage over prior art using the above methods allows automations of metrics, biometrics, thermal sensor 103 and analysis to isolate, monitor and track specific plant, animal and organisms species, using a combination metrics, biometrics, thermal sensor 103 and analysis for specific plant, animal and organism species can be monitored, charted and tracked along plant, animal and organism lifespan, unlike prior art usage of tags, radio-frequency identification (RFID) wireless non-contact use of radio-frequency electromagnetic fields identifying and tracking tags attachments, marking stickers and other manual driven mediums, the preferred method of the present invention's use of metrics, biometrics, thermal sensor 103 and analysis allows for quick and easy identification, analyzation, tracking, control and species or targeted isolation by artificial intelligence and machine learning controlled systems 624. The preferred method of the present invention therefore has the advantage versus prior art in its ability to offer a highly defined regimen for autonomy driven customized and individualized plant, animal and organisms, specific species health care plans and accompanying nutrient diet and environmental inputs with automated responses and alterations to feeds, nutrients and nutrient supplements for health monitoring, yield process accounting and quality control 601.

The preferred method of the present invention therefore has the advantage verses prior art by monetizing the additional input for optimization of the artificial intelligence control system 624 promoting robotic production, harvesting and processing efficiency for high yield aquaponics 100 and microorganism production entailing analysis, monitor, tracking and control to optimize and realize greatly enhanced production yields through enhanced plant, animal and organism species improved health, productivity from highly optimized and efficient use of energy and required inputs. The improved method of the preferred invention uses robotics to reduce exposure to the open atmosphere. The preferred method of the present invention will also reduce and potentially eliminate product loss and contaminations using the above monitoring and analysis methods to actively monitor plants, animal and microorganism species for distress, deficiencies and other contaminations with the ability to isolate, quarter and transfer for testing suspected plant, animal or microorganism species. The preferred method of the present invention gains additional advantage versus prior art by using the above methods will allow artificial intelligence and machine learning control system 624 autonomy to quickly access, determine and respond to required high production yield alterations to plant, animal and microorganism species health and optimized growth requirements for luminance levels and frequency, nutrient loading and other required environmental factors. The preferred methods of the present invention advantage over prior art will also reduce and potentially eliminate pests and insect type of contaminations and infestations.

The preferred method of the present invention advantage over prior art and its problems, issues, the preferred method of the present invention will establish levels of biosecurity and facility security not available and not found in prior art from the inclusion of the above monitoring and analysis methods, automation and robotics reduces or eliminates the above issues and others typically found with prior art facilities, practices, processes and applications. The preferred method of the present invention advantage over prior art using the above methods allows automations of biometrics, metrics and thermal analysis to isolate, monitor and track employees and guests to establish security levels of access to the facility and its food chain not available and not found in prior art farming facilities, processes or applications. The preferred method of the present invention will allow using a combination metrics, biometrics and thermal sensor 103 and analysis for any activity within the facility. The preferred method of the present invention will scan for all motions and any thermal source whether it human, animal or pest so it can be monitored, charts and tracked and recorded for historical purposes or for improper entry to alert and set alarms and record activities for actionable response or legal and criminal prosecution.

The preferred method of the present invention advantage over prior art using automation and robotics will allow a nearly closed cycle operation, using stored thermal energy for hot and cold and other temperature inputs will reduce and potentially eliminate contamination from certain types of bacteria, additional benefit from lack of uncontrolled exposure to outside unfiltered air and associated air borne contaminants. The preferred method of the present invention advantage over prior art using automation and robotics will allow a nearly closed cycle operation, prior art used suspension hooks and conveyer belts to transfer and move product between processing zones, the preferred method of the present invention uses renewable energy to provide cooling for ice flow development, using a tray system arrangement and product suspended in a slurry to reduce spoilage and bacteria exposure. The preferred method of the present invention uses one or more of the following methods to preserve animal based products include: (a) the control of temperature using ice, refrigeration or freezing; (b) the control of water activity by drying and freeze-drying; (c) the physical control of microbial loads through microwave heating or ionizing irradiation; (d) the chemical control of microbial loads by adding essential acids; and (e) oxygen deprivation, such as vacuum packing or reduced oxygen content processing areas.

The preferred method of the present invention uses an effective method of preserving the freshness of product is to chill with ice 208 by distributing ice uniformly around the product, preferably in slurry consisting of ice and water. It is a safe and highly benign method of cooling that keeps the product suspended in moisture and in easily stored forms suitable for transport. It has become widely used since the development of absorption and mechanical refrigeration, which makes ice easy and cheap to produce. Ice is produced in various shapes; crushed ice and ice flakes, plates, tubes and blocks are commonly used to cool products.

Particularly effective is when ice is used in a slurry, made from micro crystals such as those made with injection of aeration to initiate the formation of crystals of ice formed and suspended within a solution of water and a freezing point depressant, such as the addition of salt. New methods include pumpable ice technology. Pumpable ice flows like water, and because it is homogeneous, it cools the aquaculture 106 faster than fresh water solid ice methods and eliminates freeze burns. It complies with various protocols such as HACCP and ISO food safety and public health standards, and uses less energy than conventional fresh water solid ice technologies.

Because the unit operates on a substantially constant body of water which is internally recycled, large fresh water supplies are not required. Thus, the unit will be suitable for water-poor and brine heavy geographic areas and can provide an economical and plentiful source of fresh aquaculture species, fruits, herbs, vegetables and flowers not heretofore available in these regions or from off generic outdoor farming and aquatic farming season supply. The operation of the unit extremely cost efficient. The only major expenses are for fingerlings, aquaculture food, water testing supplies, and of course energy, usually in the forms of electricity, heating and cooling. However, even energy cost are minimized because in most climates a renewable energy powered and well-insulted housing will provide a highly energy efficient aquaponics facility 100.

Thermal Energy Storage (TES) 122 can be provisional via thermal energy transfer fluids and mediums generated from solar thermal and/or electrical and or chemical reaction collector systems and/or from thermal conversion is accomplished by action of chilling mechanisms, particularly special, not-compressors based, absorption chillers 131 and other devices configured to absorb, dissipate or transfer thermal energy transference into low temperature thermal energy storage 125. Additionally thermal energy can be generated via transference from a heating and/or cooling element or other derived application processes to initiate thermal conveyance to a medium, additionally as a method for electrical energy to thermal energy storage technique 122.

Thermal Energy On-Demand is made available from Thermal Energy Storage Systems 122 pumping thermal transfer fluids for direct use as a thermal energy production of a service such as providing thermal energy for a space heating, water heater or other thermal intensive applications and operations can be used to cool other units and areas within units, such as water directed to the aquaculture unit 106 or the atmosphere of the aeroponics unit 104, cold storage 127 or fast freeze storage. This process can be conducted via fluid to thermal transfer device such as a Stirling engine 126, and/or steam turbine, and/or thermal intensive applications usage and/or through a secondary thermal transfer liquid for storage and reuse of waste thermal energy.

Commercial Grid Backup Energy Reserve also called commercial grid-scale energy storage refers to the methods used to store energy on a commercial grid scale within a commercial's energy power grid. Energy is stored during times when production from energy generation components exceeds localized energy consumption and the stores are used at times when consumption exceeds available base-load production or establishes a higher baseline energy requirement.

In this way, energy production need not be drastically scaled up and down to meet momentary consumption requirements, production levels are maintained at a more consistently stable level with improved energy quality. This has the advantage that energy storage based power plants and/or thermal energy can be efficiently and easily operated at constant production levels.

In particular, the use of commercial grid-connected intermittent energy sources such as photovoltaic and thermal solar as well as wind turbines can benefit from commercial grid energy thermal storage. Energy derived from solar and wind sources are inherently variable by nature, meaning the amount of electrical energy produced varies with time, day of the week, season, and random environmental factors that occurs in the variability of the weather.

In an electrical power grid and/or thermal intensive systems with energy storage, energy sources that rely on energy generated from wind and solar must have matched commercial grid scale energy storage regeneration to be scaled up and down to match the rise and fall of energy production from intermittent energy sources. Thus, commercial grid energy storage is the one method that the commercial can use to adapt energy production to energy consumption, both of which can vary over time. This is done to increase efficiency and lower the cost of energy production and/or to integrate and facilitate the use of intermittent energy sources.

Thermal energy storage 122 most commonly uses molten salt mixture as a high temperature transfer and storage medium 121 which is used to store heat collected by a solar collection system, biogas generated thermal input or by electrical generated thermal storage injection. Thermal energy storage 122 consisting of commonly available substances and storage mediums, for example water frozen into ice to store energy as a cold temperature storage medium.

Stored energy can be used to generate electricity or provide thermal energy during inadequate solar and/or wind energy generation availability or during extreme weather events. Thermal efficiencies of 99% over one year have been predicted. Thermal Energy Storage System 122 has shown that the electricity into storage to electricity-out (round trip efficiency) in the range of 75 to 93% using enhanced energy recovery systems.

Electricity generated by the onsite power generation unit is used to operate all Electrical devices needed to ensure proper operation of the production and cold storage system 127. Electricity is transferred using common electrical conduits and means of conduction electrical energy. Any excess electrical energy produced by the onsite power generation unit can be sold to the local utilities through a direct utility connection and monitors.

Therefore, borne out of necessity is the creation of mechanism for mitigating variability and/or intermittency associated with the stable quality power production of energy consisting primarily of energy from wind, photovoltaic solar, thermal solar and other renewable energy sources, additionally the absence of adequate solar energy generation for thermal solar energy with the purpose of thermal energy availability.

It is a principal object and advantage of the present invention to maximize renewable energy as opposed to grid connected fossil and nuclear fuel sources for aquaponics systems 100. Renewable energy is a term of art used to describe power derived from environmentally friendly sources of energy including renewable (or regenerative), non-polluting energy sources. (No source can be completely non-polluting, since any energy source requires an input of energy which creates some pollution.) Specific types of renewable energy include wind power, solar power, hydropower, geothermal power, and biomass/biofuel power.

It is another object and advantage of the present invention to use renewable energy sources instead of non-renewable energy sources in a trigeneration system set-up to create the electrical energy, heating and cooling to operate an aquaponics system 100, which can greatly reduce the costs in operating and maintaining such a system. Trigeneration CCHP (also known as combined cooling, heat and power) refers to the combined production and utilization of electricity and heat energy, where the heat energy such as biogas conversion for CO2 generation would normally be wasted, from a common fuel source.

This “waste heat” is typically created as a byproduct during an industrial process. Instead of releasing this heat into the surrounding environment (and essentially treating this heat energy as waste heat), a trigeneration system will harness this heat energy for further thermal storage input and future uses. Such uses would include absorption cooling 124 for refrigeration and cold storage 127. Trigeneration systems allow for the use of a higher percentage of energy obtained from an energy source. This translates into energy conservation, and thus savings to the user of the trigeneration system, since less of the energy needs to be used to obtain the same amount of useful energy from the energy source (as compared to a system that does not harness the waste heat).

The preferred method of the present invention sets forth its primary advantage and novel method over prior art above applications and processes with physically connected preheaters and heating system elements, heat exchangers 129 and regenerators in its reclamation and recycling of waste thermal energy for use, reuse, storage and/or conversion and storage. This energy is used by thermal intensive applications such as with stirling cycle engines 126 which use a portion of the thermal energy for the generation of rotational energy, for use in such applications such as rotation work needed for input into a generator, pump or compressor. Waste heat recycled from this process may be used in a second level of reuse of available waste energy as thermal energy input into secondary lower heat threshold thermal intensive applications such as stirling engine cycle 126 with a reduced temperature differential which would then use a portion of the thermal energy input for the generation of rotational energy for us in such applications such as rotation work needed for input into a generator, pump or compressor.

The present invention has additional advantage over prior art from additional applications and process cycles from remaining thermal energy and from storage to further encourage use and recycling of available energy for additional application and processes energy usage that may be added based on available input temperatures and return on investment cost versus an acceptable benefits to costs ratio, all remaining recyclable thermal energy may then be reclaimed and then communicated to appropriate temperature thermal storage systems, additionally thermal energy may be communicated to absorption cooling 124 to convert heat based thermal energy into cold based thermal energy to maintain a localized energy balance of available stored thermal energy. The preferred method of the present invention sets forth its primary advantage and novel method over prior art provides for normalized thermal energy balance that is essential for realized and optimized system wide use and reuse efficiencies concurrently monetizing all energy inputs for all intended applications and processes.

Single cycle and multiple cycle generation systems using steam turbines or stirling engines 126 as the primary thermal energy cycle and for additional benefit use of recycled thermal waste energy for additional thermal intensive applications such as additional stirling engine stages 126 may be used for additional benefit and enhanced efficiency.

The preferred method of the present invention reduces and potentially eliminates these issues with its energy generation, extremely high volume energy storage system 122 and finally its ability to capture and recovery waste heat for the purpose of communication to energy storage and/or for conversion too cold to cool the system all of which ULTRAGRID™ 700 can provide analysis, monitoring and control of any and all available energy and potential energy needs as depicted in FIG. 6 discussed above.

The preferred method of the present invention has the additional benefit from connection to thermal energy storage 122 for the purpose of preheat of primary thermal energy input which then offers the included ability to communicate recycled and recovered thermal energy for the purpose of thermal energy storage 122 or reuse, this offers the advantage over prior art in it gains the system higher efficiency and reduces energy input requirements with inclusion of renewable energy generation and associated thermal and gas emissions processing and storage. There preferred method of the present invention has the additional advantage over prior art in its ability to reduce reliance on fossil fuels and non-green energy input sources

The preferred method of the present invention advantage over prior art will be appreciated with energy input from localized energy storage 122 that will provide energy thermal input to on demand energy generation provisioning versus prior art that required external grid energy generation input that's source generally was hundreds of miles away all points of failure and efficiencies and losses associated. The preferred method of the present invention advantage over prior art from localized energy generation and enhanced duration of localized energy storage 122 available only from the preferred method of the present invention use of thermal energy storage 122 for generation of energy to facilitate fulfillment of present and future energy needs with on demand and when needed energy provisioning. The preferred method of the present invention advantage versus prior art that required external grid energy generation input that's source generally was hundreds of miles away all points of failure and efficiencies and losses associated with prior art processes and applications versus the preferred method of the present invention use of locally generated and/or stored energy provisioned on a on demand or as needed basis via ULTRAGRID™ 700 that can provide analysis, monitoring and control of any and all available energy and potential energy needs for mission critical reliability with on demand or as needed basis.

The efficiency of a trigeneration system increases when the heating or cooling that is obtained from an energy source is utilized close to where the heating or cooling is created and harnessed. Further, the heat energy can be in the form of hot water or steam when not used for space heating, for example. It is a further object and advantage of the present invention to exploit such renewable energy in a trigeneration facility, where the renewable energy could be utilized to its fullest potential thereby using less energy and passing off the savings to the user of such a facility.

It is another object and advantage of the present invention to provide an aquaponics system 100 that is grid independent and can operate almost anywhere (e.g., an open lot in a city or a field in the country), and can allow food to be grown close to customers, eliminate transportation costs, enhance food safety by growing food in a controlled environment, recycles wastes, and helps conserve resources such as soil, water and wild fish populations.

In accordance with an embodiment of the present invention, aquaponics system 100, and, more a particularly, combined interdependent fish and plant factory that creates sources of renewable energy, is powered by renewable energy, and utilizes waste heat and CO2 generated by the enclosed digestion unit systems. An embodiment of the present invention combines fish farming, aeroponics vegetable cultivation 104, and energy renewable energy production and storage.

In accordance with preferred embodiment of the present invention, a multi-building, multi-level, soil-less, climate controlled, aquaponics system 100 produces aquaculture products 106, aeroponics products 104, heating, cooling and electricity is provided. a combined interdependent aquaculture and horticulture plant factory comprising an aquaculture 106 with a plurality of aquaculture tanks adapted for containing water and aquaculture species therein, and a greenhouse with a plurality of aeroponics tanks adapted for containing plants in grow beds therein, within a multi- building, vertical stacked, multi-level housing unit, is provided.

In accordance with a preferred embodiment of the present invention, the aquaculture portion 106 if the overall structure of the embodiment of the present invention is connected to the hatchery and interconnected to a harvesting and processing building 134. The aquaculture system 106 is preferably adapted for excluding sunlight and maintaining a relatively constant temperature for the aquaculture tanks. The aeroponics tanks, in turn, are preferably housed in an adjacent greenhouse but can form the other portion of the overall structure a combined aquaponics system 100 of an embodiment of the present invention.

In accordance with a preferred embodiment of the present invention, the aquaponics system 100 of an embodiment of the present invention comprises a duct with a plurality of ducts that can connect the aeroponics tanks of the greenhouse with the aquaculture tanks or microalgae bioreactor 130, organism reactor 132 and with the filter feeder system 136. This connection is for the purpose of circulating water through the system with the assistance of at least one pump. The aquaponics system 100 operates on a substantially constant body of water that is continuously circulated or recycled (as described supra) from the aquaculture tanks through at least one filter (e.g., a bio-filter i.e. species and/or processes for converting ammonia to nitrite and nitrite to nitrate) and/or filter feeder system 136 and/or digester units 112 and then finally back to the aeroponics tanks and back again.

Referring back to FIG. 14, the method of operation for the water supply of the system 100 is shown. With the assistance of the at least one pump, aquaculture effluent, such as nitrogenous wastes, are removed from the aquaculture tanks and are provided to the plants in the grown beds from water in the aeroponics tanks. These nitrogenous wastes, as noted supra, act as one of the renewable sources of nutrients for the plants, while the and the filter feeder system 136 and/or plants serve as a filter to recycle the water for the aquaculture 106. The plants effectively maintain the aquaculture water purity in a habitable condition by removing these wastes which would be toxic to the aquaculture 106. In essence, the water is reused, filtered and sterilized while the fish and plants are grown in a controlled environment.

In accordance with an embodiment of the present invention, the aquaculture system 106 is adaptable to growing any number of a wide variety of aquatic species referred to herein simply as fish. The aeroponics system 104 is adapted from growing plant life, and most preferably plants which produce herbs, fruits, vegetables and flowers. In a preferred embodiment of the present invention, no pesticides of any kind are used on the plants. Thus, the plants and fish grown in accordance with the present invention may be able to be certified “organic,” provided that they meet other requirements of such certification.

In accordance with an embodiment of the present invention, Stirling engines 126 are provided for electrical energy to the aquaponics system 100 of an embodiment of the present invention. These Stirling engines 126 can run on any available thermal waste energy, thermal energy storage 122 or thermal generator for example biogas burner 116 and 122. These Stirling engines 126 that utilize the collected or stored thermal energy create renewable “green” energy (electric power), and provide the electric energy to the aquaponics system 100 of an embodiment of the present invention for many purposes. These purposes include running the pumps to circulate the water from the aquaculture tanks to the aeroponics tanks, and powering other devices including any lighting provided in the aquaculture facility 106 as well as other operating units within the factory. This electric power can also be provided to a substation and to a power grid to power other facilities.

In accordance with an embodiment of the present invention, the Stirling engines 126 are connected to waste heat recovery heat exchangers 129 (in a combined heat and power set-up) which harness the waste heat from the Stirling engines 126 and provide this waste heat energy in the form of steam and/or hot water to the absorption cooling 124 or heating to the aquaponics system 100 of an embodiment of the present invention for optimum growth/yield of the fish and plants within the system (e.g., heat the aquaculture tanks and heat and cool the greenhouse).

This waste heat energy can also be provided in the form of steam and/or hot water to other facilities, such as a passive floor and wall heating digester unit 112 pre-heating. This combined heat and power set-up can increase the energy efficiency from about 35% (without the use of a combined heat and power set-up) to about 70-90%. Additionally, CO2 and fertilizer created during the included digester 112 or biogas burner processes 116 is also harnessed and provided to aquaponics 100 of an embodiment of the present invention for purposes such as photosynthesis and optimum plant growth/yield. This CO2 enhances the atmosphere of the greenhouse where the plants capture the carbon generated in this process.

In accordance with an embodiment of the present invention, aquaponics waste created by the aquaponics system 100 of an embodiment of the present invention can be provided to the compost system for the production of CO2 and fertilizer for the said aquaponics system 100. Additionally, a microalgae bioreactor 130 can be provided as part of the aquaponics system 100 of an embodiment of the present invention. The microalgae reactor can utilize the waste heat energy in the form of steam and/or hot water from the waste heat boilers, as well as the CO2 created during the digester activity 112 and biogas burner process 116.

The present invention consists primarily of a renewable energy system based microgrid CCHP for electrical energy, heating, cooling and energy storage 122. The present invention consists of robotic based automation of processes and applications for an integrated hybrid aquaponics system 100 consisting of an aquaculture section 106 and aeroponics section 104. The present invention consists of artificial intelligence and machine learning based automation of processes and applications 624 for an integrated hybrid aquaponics system 100 consisting of an aquaculture section 106 and aeroponics section 104.

Several embodiments of the invention advantageously address the above needs as well as other needs by providing an aeroponics/aquaculture system 104 and 106 that is smart, sustainable, efficient, productive in both crop yield and human, factors, automated, conserves water, minimizes environmental impact, optimizes wildlife habitat, minimizes pollutant generation, such as nitrogenous and organic carbon wastes, and reduces energy consumption, and its related methods.

In one embodiment, the invention can be generally characterized as an aquaponics system 100 for use with a plurality of growth reservoirs containing a fluid, comprising a main control unit for receiving feedback, further novel method would include using adaptive biometrics, thermal imaging sensory and additional sensors for detection product contaminations, product quality assurance tracking 616 of all methods, applications and product, can be quickly and easily be identified and providing additional information for the control system and providing control, at least one pump control unit in electronic communication with the main control unit, and at least one gate control unit in electronic communication with the main control unit.

In an alternative embodiment, the invention can be generally characterized as a method of fabricating an aquaponics system 100 for use with a plurality of growth reservoirs containing a fluid, the method comprising providing a main control unit for receiving feedback, further novel method would include using adaptive biometrics, thermal imaging sensory and additional sensors for detection product contaminations, product quality assurance tracking 616 of all methods, applications and product, can be quickly and easily be identified and providing additional information for the control system and providing control, providing at least one pump control unit in electronic communication with the main control unit, and providing at least one gate control unit in electronic communication with the main control unit. In yet another embodiment, the invention can be generally characterized as a method of using an aquaponics system 100 for use with a plurality of growth reservoirs containing a fluid, the method comprising: providing an aquaponics system 100, the system providing step comprising providing a main control unit for receiving feedback and providing control, providing at least one pump control unit in electronic communication with the main control unit, and providing at least one gate control unit in electronic communication with the main control unit powering the system and running the system.

Energy generation, processing and energy storage 122 with a complimentary shared computerized data system using a common data interface into element subsystem and interconnecting backbone network with an interactive artificial intelligence control and management system 624 providing intelligent energy provisioning based on past usage and intelligent projected energy generational needs. The invention is contemplated for use as a fully integrated distributed renewable energy ecosystem for a flexible interconnected energy system solution, providing energy generation for electrical power generation, thermal energy for thermal storage and thermal intensive consumer usage.

The object of the present invention is to provide ultra-high density with ultra-high yield aeroponics 104, aquaculture 106 and bioreactor biomaterial production facilities powered by efficient combined heating and cooling, electrical energy and CO2 neutral generation, capture and recirculation technology, including using waste bio material streams as a primary source of digester processes 112. Furthermore, the present invention is directed to enhance aeroponics 104 and aquaculture production 106 by the inclusion of CO2 capture from the digester unit 112 facilities as well as the aquaculture facilities 106 and delivery technologies that create a semi-closed loop production facility.

The advantages of using a controlled aquaculture production system 106 are readily apparent. By using the internal waste streams of a combined aeroponics 104 and aquaculture system 106 to complement external waste sources, the present invention provides inputs to a digester unit 112 for processing, useful by-products can be provided and recovered where and when necessary and expensive by-product waste removal can be reduced to minimal levels. It is the principal object of the present invention to link a renewable form of energy and CO2 generation with energy and resource intensive good production facilities. The present invention achieves increased, high yield food production, by interlinking land based fisheries and farms that is more integrated than those systems presently know in the art. The present invention broadens the scope of the foods (both plant and aquaculture 106) that are capable of being produced.

Additionally, the present invention allows for significant increases in production, both in terms of size and harvest cycles, by controlled capture and use of carbon dioxide and other regulated control inputs. Furthermore, by incorporating renewable energy based on onsite generation and storage, including municipal bio-material waste streams, it allows the products to have a more readily acceptable from economic viability of commercial adoption due to lower energy costs due to renewable energy savings, renewable energy subsidies and carbon capture credits. The present invention is a controlled intensive, high yield production system that allows for land-based aquaculture 106 and aeroponics production facilities 104 to be connected to a digester processing unit 112. Through this connection, electricity, CO2 and heat are transferred via the aquaponics production facilities 100.

In turn, aquatic waste is transformed to a nitrogen-based, liquid-form fertilizer for reuse in the aeroponics nutrient system, with usable residual waste used as nutrient rich potting and planting soil for an additional revenue channel. As a by-product of using external and/or municipal and/or aquatic waste, the digester unit system 112 produces excess water. Once filtered, this water can then be reintroduced into the aquaculture facility 106 or into an aeroponics facility 104. An additional by product of the digester process 112 is the production of carbon dioxide (CO2) and methane. The burning of the biogas and capture of CO2 is sequestered during production and transferred, along with electricity, to the aeroponics facilities 104. The aeroponics facilities 104 themselves are sealed to maintain a desired ambient concentration of CO2 and other gases to optimize plant growing conditions.

Multiple different aeroponics facilities 104 can maintain different various required levels of CO2 in the controlled and sealed growing space. Through this increased CO2 atmosphere, the aeroponics system 104 is able to achieve a higher yield than would be available with a standard aeroponics 104 or soil-based system. The system also includes CO2 recapture technology to restore CO2 levels to normal prior to introducing human workers into the sealed and controlled growing areas.

The aeroponics system 104 in turn transfers aeroponics waste that is filtered and provided either as partial feed for fish or fuel for the digester unit system 112 or as raw inputs to the aeroponics system 104. The present invention is also directed to a novel method of producing high yield aeroponics 104 and aquaculture products 106 by using a combined aquaculture 106 and aeroponics system 104 to managing the introduction of gases, particularly CO2, into a sealed and controlled aquaponics facility 100. The gas management method may also include capture and concentration of oxygen from aquaculture respiration for compression and filtration for potential use in producing ozone for enhanced purification of recycled water. The preferred invention relates to aquaculture farming systems 106 and effluent, waste or water treatment systems which are generally referred to as aquaculture systems 106 in this specification.

The present invention uses plastics, metals, and water resistant materials to encourage nearly unrestricted support of the plant to allow for above normal growth in the air/moisture/nutrient environment. Air gaps optimize access of air to roots for healthy roots and assist plant growth. Materials and devices which hold and support the aeroponics grown plants must be devoid of disease or bacteria and pathogens. A distinction of a true aeroponics growth mediums and apparatus is that is provides plant support features that are minimally invasive.

Minimized contact between a plant and support structure allows for extremely high percentage of the plant to have access to air. Aeroponics cultivation 104 requires the root systems to be free of constraints surrounding the stem and root systems. Physical access and contact is minimized so as to not suppress natural growth and root expansion or access to water, nutrients, air exchange and disease-free conditions.

Benefits of oxygen in the narrow region of the root zone also known as rhizosphere is required for healthy roots and stable plant growth. Further advantage of the present invention may be realized through the use of aeroponics 104 which is orchestrated in atmospheric air combined with a spray of nutrients suspended in water micro-droplets, almost any plant can grow to maturity in air with a plentiful supply oxygen, carbon dioxide, water and nutrients which may then be actively analyzed, monitored and controlled via adaptive biometric and thermal imaging analysis 604, monitoring and control through the use of artificial intelligence and machine learning control system 600 for constant optimization of species health and growth.

Present invention favors aeroponics systems 104 over other methods of hydroponics because the increased aeration of nutrient solution while delivering reducing the delivered amount of nutrient solution used and providing more oxygen to plant roots, stimulating growth and helping to prevent pathogen formation and damage from too much moisture such as root rot and other root diseases.

Water droplet size is crucial for sustaining aeroponics growth. Too large a water droplet means less oxygen is available to the root system. Too fine a water droplet, such as those generated by the ultrasonic mister, produce excessive root hair without developing a lateral root system for sustained growth in an aeroponics system 104. Ultrasonic units can also suffer from electrical malfunction. Mineral buildup of valves and clogging of sprayers in the normal use of ultrasonic transducers requires scheduled maintenance for dependable operation and yet the potential for component failure. This is also a shortcoming of metal spray jets and misters. Restricted access to the water causes the plant to los turgidity and wilt. The preferred method of the present invention has the advantage using adaptive biometrics, thermal imaging sensory and additional sensors for detection that if a particular plant does become distressed from lack of water or nutrients, the sensory input can be used to quickly identify the plant and/or plants in question and address the issue quickly and effectively before product loss occurs.

Within the aeroponics growth cycles 104, the deleterious effects of using seed stocks that are infected with atmospheric and soil based pathogens can be minimized due to the separation of the plants and the lack of a shared common growth matrix. Additionally aeroponics 104 can be an ideal growth system in which to grow seed stocks that are nearly pathogen-free and potentially more important in some cases seed stock growth is removed from atmospheric contamination from crops, consisting of GMO genetic modified organisms. The enclosing of the growth chamber, in addition to the isolation of the plants from each other discussed above, helps to both prevent initial contamination from pathogens introduced from the external environment and minimize the spread from one plant to others of any pathogens that may exist.

Lastly another improvement and advantage over prior art is that the preferred invention with its enclosed nature and closed filtered air handling system is the enhanced ability to shut contamination from external GMO's (genetically modified organism) crops while sustaining each seed and plant with its uncontaminated organic and natural qualities and retaining worldwide acceptance.

Aeroponics 104 can limit disease transmission since plant-to-plant transmission is greatly reduced and each spray pulse can be sterile. In the case of soil, aggregate, or other media, disease can spread throughout the growth media, infecting and potentially reinfection of other plants. Generally in typical greenhouses, the solid media require sterilization methods performed after each crop harvest 410 and, in many instances, the expensive media is simply discarded and replaced with fresh, sterile media.

A distinct advantage to reduce additional contaminations of aeroponics technology 104 is that using adaptive biometrics, thermal imaging sensory and additional sensors for detection that if a particular plant does become diseased, it can be quickly be identified and removed from the plant support structure without damaging, disrupting or cross infecting the other nearby plants.

The present invention offers a dust free and disease-free filtered and metered environment that is unique to prior art aeroponics, many plants can grow at higher density and higher yield (plants per square meter) when compared to more traditional forms of cultivation such as typical soil based agriculture or hydroponics with its two main types are solution culture and medium culture. Solution culture does not use a solid medium for the roots, just the nutrient solution.

The three main types of solution cultures are static solution culture, continuous-flow Solution culture and aeroponics 104. The medium culture method has a solid medium for the roots and is named for the type of medium such as gravel or rockwool. There are two main variations for each medium, sub-irrigation and top irrigation. For all techniques, most hydroponic reservoirs are now built of plastic, but other materials have been used including concrete, glass, metal, vegetable solids, and wood. The containers should exclude light to prevent algae growth in the nutrient solution.

Aeroponics products are highly perishable foodstuff which needs proper handling and preservation to have a long shelf life and retain a desirable, visually attractive quality and nutritional value. The central concern of aeroponics products processing is to prevent products from deteriorating which leads to excessive waste removal and product loss. The most obvious method for preserving the quality of aeroponics product is to keep them cool, and crisp until processing and packaging. Other methods used to preserve aeroponics products include: (a) the control of temperature using ice, refrigeration or freezing; (b) the control of water activity by drying and freeze-drying; (c) the physical control of microbial loads through microwave heating or ionizing irradiation; (d) the chemical control of microbial loads by adding essential acids; and (e) oxygen deprivation, such as vacuum packing or reduced oxygen content processing areas.

Usually more than one of these methods is used conjunctively, further novel method of the present invention would include using adaptive biometrics, thermal imaging sensory and additional sensors for detection product contaminations, product quality assurance tracking 616 of all methods, applications and product, can be quickly and easily be identified. When chilled or frozen aeroponics products are transported by pallet, forklift, conveyor belt, road, rail, sea or air, the cold chain must be maintained at all times. This requires insulted containers or transport vehicles and adequate refrigeration. Modern shipping containers can combine refrigeration with a controlled atmosphere. Aeroponics processing 104 is also concerned with proper waste management and with adding value to aeroponics products and potential use for nutritional input for manufacturing of enriched pet foods.

### Air Flow Circulation System

Air flow system consists or at least one ventilation fan, heating and cooling exchanger, dehumidifier and humidifier unit, pressure swing unit and CO2 system using artificial intelligence control and adaptive machine learning 624 for maximum efficiency and promote high yield facility production. First half of paragraph moved to end of details and second half moved to before paragraph 331

An air recirculation system is provided for controlling the air temperature, humidity and gas composition of the air within the growing chamber. The air recirculation system includes fans and/or blowers for circulating and moving air flow through the growing chamber, as shown in and a controlled exhaust and intake means may be provided for bringing fresh air into the growing chamber and exhausting air from the growing chamber as required. The humidity of the air within the growing chamber may be controlled by air heating and cooling heat exchangers 129. Water collected in the humidity control system may be used for growing by addition to the nutrient solution. IF moisture is needed to increase the air humidity, ultrasonic sensors may be used to create very small water particles, increasing humidity. Hydrogen peroxide in a low concentration may be used in the air recirculation system as a disinfectant and to kill bacteria. The preferred method of the present invention uses an air supply system that preferably includes an artificial intelligences and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system.

Because the growing chamber is a sealed unit, pests and diseases are easily controlled and, with added protection, the unit according to the present invention can eliminate chances of contamination. air in the optimized environmental recirculation system will pass through an ozone generator and an ultraviolet light to kill any spores or bacteria in the air, also eliminating odors using monitoring which includes an artificial intelligence and machine learning control system 624 with active interfacing of adaptive biometrics, thermal imaging sensory and additional sensors for detection product contaminations, product quality assurance tracking 616 of all methods, applications and product, can be quickly and easily be identified and analyzed to provide additional information for the control system.

Production of thermal energy is based on the premise that fluctuation of generational inputs is acceptable due to inherent design adaptations that maximize production during high energy generation availability and can scale downward or enter standby mode to match input limitations from lower generational capacity periods.

However, generational output of the renewable energy technologies may fluctuate from inherent variations in environmental changes and effectual actions. Furthermore, such fluctuations may prevent the renewable energy generation technologies from balancing energy generation with energy demand (e.g., grid electrical demands, thermal applications and components).

As a result, the system may incur costs associated with operating and/or shutting down electric generators powered by other forms of energy (e.g., hydrogen, ammonia, thermal, coal, natural gas, hydroelectric power, nuclear power) in response to changes in electric demand and/or fluctuations in the supply of renewable generated power.

To reduce such costs and/or increase the reliability of renewable power, the system of FIG. 1 may store energy from the renewable energy generation and subsequently generate energy in the form of electrical and thermal, hydrogen and ammonia from the stored energy based on electric demand. First, the energy may be stored in a chemical storage system such as hydrogen, ammonia and other stored gases (e.g. Argon, Helium, Neon, etc.).

Second, the energy may be stored as heat in a high-heat-capacity thermal storage system (e.g. molten salt, etc.). Low-heat-capacity working fluid may additionally be placed into an insulated storage vessel to retain the heat in short term stored low-heat-capacity fluid and/or to use external thermal input to maintain usable low-heat-capacity fluid capability.

To generate electricity from the stored energy, a chemical-transfer mechanism, energy generation may selectively transfer chemical from storage to provide on demand energy generation.

Additionally heat-transfer mechanism, energy generation may selectively transfer heat from thermal storage to provide on demand energy generation. Heat energy without conversion can be used to initiate stirling engine 126 thermal energy input. Once heat is transferred, heat may also boil a working fluid (e.g., due to the low boiling point of working fluid), generating and steam and/or vapor that is used to rotate rotor blades of a turbine. Turbine and/or stirling engine 126 usable work energy may then be used to drive an electric generator that supplies electricity to a load, or other uses for example such as providing rotational and/or linear energy for a pump or compressor and/or thermal energy to a thermal intensive application.

such on-demand generation of energy from stored renewable energy may additionally reduce cost associated with the operation of other power stations to offset fluctuations in energy generation from renewable energy. Along the same lines, the use of mechanical elements (e.g., rotation-transmission mechanism and/or linear transmission mechanism and/or specifically could be rotor blades and/or gas and/or working fluid activated pistons), low-heat-capacity fluid and friction to store the energy may provide cost saving over conventional energy storage mechanisms such as batteries and/or pumped-storage hydroelectricity. In other words, the system of FIG. 5 may facilitate the effective, economical, and/or reliable generation of electricity and other thermal intensive applications with renewable energy.

FIG. 5 show heat-transfer mechanism in accordance with an embodiment. As mentioned above, heat-transfer mechanism may enable the selective transfer of heat from low-heat-capacity fluid to working fluid. Heat-transfer mechanism and/or device may include a thermally conductive component such as a thermally insulated pipe and a thermally insulating component. Thermally conductive component may include a metal surface, manifold, conductive rod, radiator, and/or other structure that facilitates heat transfer mechanism. Conversely, thermally insulating component may include a vacuum-insulated panel and/or other Thermally insulating material or structure.

To retain heat in low-heat-capacity fluid, thermally insulating component may be positioned between low-heat-capacity fluid and working fluid, as shown in FIG. 5. (Note that the positions of components and may be interchanged.) Because low-heat-capacity fluid is also enclosed in an insulated vessel (e.g., thermal insulated storage vessel of FIG. 5), energy may be effectively stored in low-heat-capacity fluid as long as thermally insulating component prevents low-heat-capacity fluid from thermally contacting thermally conducting component and/or working fluid.

To transfer heat from low-heat-capacity fluid to working fluid, thermally insulating component may be redirected to enable thermal contact between low-heat-capacity fluid and working fluid through thermally conducting component. Once thermal contact is made between low-heat-capacity fluid and thermally conducting component, heat may be transferred from low-heat-capacity fluid to working fluid.

FIG. 5 shows a flowchart illustrating the process of generating rotational and/or linear energy to provide usable work torque, for example to activate a pump or generator in accordance with an embodiment. In one or more embodiments, one or more of the steps may be omitted, repeated, and/or performed in a different order. Accordingly, the specific arrangement of steps shown in FIG. 5 should not be construed as limiting the scope of the embodiments.

Next, an insulated pressure vessel may be used to retain heat in the low-heat-capacity fluid. The rotating blades and insulated vessel may thus facilitate the storing of energy from the renewable energy in the low-heat-capacity fluid. The stored energy may then be used to generate electricity and thermal energy based on energy demand associated with energy requirements.

To generate electricity from the stored energy, the chemical and/or heat from the associated storage of low-heat-capacity fluid may be selectively transferred from the low-heat-capacity fluid to the working fluid. For example, a thermally insulating component may be disposed between the low-heat-capacity fluid and the working fluid to retain the heat in the low-heat-capacity fluid. During periods of low solar and/or low wind and/or high electrical demand, the thermally insulating component may be repositioned to transfer the heat from the low-heat-capacity fluid to the working fluid through a thermally conductive component such as a metal surface, a manifold, a conductive rod, and/or a radiator.

Finally, the transferred heat in the working fluid is used to generate electricity. More specifically, the working fluid may be associated with a low boiling point, such that the transfer of heat from the low-heat-capacity fluid to the working fluid quickly boils the working fluid. Vapor and/or Steam from the boiled working fluid may then be used to rotate a turbine's rotor blades, and the turbine may be used to drive a rotational device for usable work.

The preferred embodiment for the hybrid energy generation system consists of two core elements, one element consists of the thermal solar energy collection modules with an associated centrally located absorber for thermal collection and the other element is the thermal energy storage system 122 for quintessential heat and cold based storage.

The preferred embodiment for the central thermal solar system is modular design construction, consisting of row of rectangular panels with parabolic shape and a central axis on each row, giving them the ability to track the sun and focus reflected light onto the closest absorber.

The preferred embodiment for the horizontally mounted thermal solar absorber 128 consists of pipe like structure to be mounted parallel above the horizontally mounted solar panel segments and absorb the focused solar energy from the panels below. The absorber will itself also has a rectangular panel with parabolic shape mounted above the absorber to cause reflected solar energy from the below panels that extends past the absorber to be reflected upon the top of the absorber to cause efficiency enhancement with a nearly 360 degree solar contact upon the absorber surface.

The disclosed embodiments provide a method and system for generating thermal energy in the form of thermal heat energy or communicated to a chiller and/or cooling process for cold based thermal storage. A solar power from solar collection system, wind power may be collected by a wind turbine, geothermal power may be collected from a geothermal power plant, hydroelectric power may be collected from a hydroelectric power generation source or grid connected to collect power from available grid energy sources.

The preferred embodiment for the Thermal Energy Storage (TES) system 122 consists primarily of a high temperature storage vessel 121, medium temperature storage vessel 123 and low temperature storage vessel 125. Additional improvement is the addition of a forth thermal storage vessel consisting primarily for hot water storage 120 that doubles as a waste energy thermal storage.

The preferred embodiment uses high temperature stored thermal energy 121 as energy input for an ammonia based cooling process to initiate and provide temperature support energy for low temperature storage vessel energy input 125. The preferred embodiment also uses high temperature stored thermal energy 121 as energy input for a heating process to initiate and provide temperature support energy for space heater, room, area or building heating system. Additionally, the preferred embodiment uses low temperature stored thermal energy 125 as energy input for an active cooling process to initiate and provide temperature support energy for central air conditioning and cooling. The preferred embodiment also uses low temperature stored thermal energy 125 as energy input for an active cooling process to initiate and provide temperature support energy for refrigeration appliances, walk-in refrigerators, wine storage areas, box and water cooling. The preferred embodiment further allows the use of low temperature stored thermal energy 125 as energy input for an active cooling process to initiate and provide temperature support energy for freezer appliances, walk-in freezers, and/or box freezers.

The preferred embodiment consists of a stirling cycle 126 using the available stored high temperature thermal energy 121 to initiate gas and/or working fluid expansion for the generation of rotational and/or linear movement. The preferred embodiment comprising of a stirling cycle 126 uses the available stored low temperature thermal energy 125 to initiate gas and/or working fluid contraction for the generation of rotational and/or linear movement. It uses generated rotational and/or linear movement applied to a generator for the production of electrical energy. It can also use generated rotational and/or linear movement applied to a pump or compressor for the pressurization and communication of liquids, gases and/or working fluid. Furthermore, the preferred embodiment uses recycled thermal waste heat from the stirling cycle 126 as energy input for a heating process to initiate and provide temperate support energy for space heater, room, area or building heating system. The preferred embodiment may also use recycled thermal waste heat from the stirling cycle 126 as energy input for a heating process to initiate and provide temperature support energy for water heating application. Another embodiment with less efficiency and not optimum performance would entail the usage of a steam engine in place of a stirling process engine 126.

### Energy System Artificial Intelligence

Artificial Intelligence Management System (AIMS) 624 integration provides software and hardware based integrated control, data acquisition and processing for grid management, energy generation system, hydrogen generation system, ammonia production system, energy regeneration system, performance tuning, power monitoring 714, frequency matching and control system redundancy. This is combined with machine learning for automated maintenance scheduling 622 for enhance uptime availability. The system additionally offers a secured SCADA integration solution for data interfacing for local and remote visual overview, monitoring and control.

Additionally the system provides active condition monitoring of system components and sensors for health monitoring, identify changes and trends to optimize overall performance, monitor alert levels, update and contact maintenance of pending issues for a proactive maintenance scheduling 622 approach before faults occur.

Commercial Grid management system integration provides intelligent control of energy generation for load matching and projected requirements of the load generation system for higher generated energy utilization. Active monitoring and control of regeneration energy systems for backup and base load provisioning to prevent brownouts from lack of energy generation availability, Smartgrid interfacing and monitoring for energy generation and energy use projections.

Energy generation system integration provides intelligent interfacing of generation systems and load provisioning systems. Interaction of data between systems allows stable grid power control with less power spikes while increasing uptime availability promoting maximum efficiency of energy processing and storage systems.

Energy storage 122 locally integrated bridges communication from energy generation sources to intragrid control for power conversion based on variable input energy to thermal storage systems 122. Energy storage system 122 integration enables maximum energy generator with optimized energy collection. Mission critical response times for the highest efficiency and safety levels. Thermal energy to electrical and thermal energy on demand for thermal intensive applications integration allows timely and responsive energy generation capabilities to respond to heavy baseline load requirements and needs based on smartgrid communications.

ULTRAGRID™ system integration 700 allows fast interaction of energy systems for maximum power availability and flexibility to handle all system needs and energy requirements. This integration extends the compatibility and usability into additional initial end user product design and manufacturing.

The description in the above sections and the following is presented to enable any person skilled in the art to make and use the embodiments, and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present invention is not limited to the embodiments shown, but is to be accorded the widest scope consistent with the principles and features disclosed herein.

Thus, the present invention has been described in an illustrative manner. It is to be understood that the terminology that has been used is intended to be in the nature of words of description rather than of limitation. Consequently features specified in one section may be combined with features specified in other sections, as appropriate.

Many modifications and variations of the present invention are possible in light of the above teachings.

The foregoing descriptions of various embodiments have been presented only for purposes of illustration and description. They are not intended to be exhaustive or to limit the present invention to the forms disclosed.

Accordingly, many modifications and variations of the present invention are possible in light of the above teachings will be apparent to practitioners skilled in the art. Additionally, the above disclosure is not intended to limit the present invention.

An important feature of the present invention is the method of feeding the fish both by individual species as well as potentially within poly-culture settings. For example. tilapias are omnivores that prefer a plant based diet, while hybrid striped bass omnivores that strongly prefer a carnivorous diet. Regarding the present invention, aquaculture 106 is typically fed the following foods as listed below along with its nutritional content:
1) Blue-green algae: this is naturally occurring, essential food source. Blue-green algae deliver omega-3 essential fatty acids to their aquatic consumers. The algae are regularly managed from a waste to metabolic removal interval to ensure the highest nutritive value for our aquaculture species as well as the most efficient metabolic waste removal from the water system. Blue-green algae delivers up to 61% protein to the fish, and since threadfin shad 138 and freshwater sardine 138 are filter feeders, they essentially are eating the blue-green algae every time they breathe.
2) Harvested edible grasses, weeds and plant roots that can be an important source of good nutrition for the aquaculture species.
3) Water Lettuce: this tropical plant is a good food additive choice for aquaculture 106 and offers protein content around 24% mark.
4) Spinach is commonly known to grow quite quickly and contains decent amounts of nutrients while low in protein content. Spinach can be sold on the open markets, thus an ongoing on a cost-basis feeding it to the aquaculture should be done sparingly.
5) Duckweed: this native plant is a tremendous asset to an aquaponics system 100. Duckweeds's protein content can exceed 35% and with the appropriate nutrient base, these plants can double its size every day.
6) Filamentous Algae: when careful management, this native plant can also be very useful. Aquaculture tends to eat it aggressively, and the protein content can range into the 25-35% area.

It is important to note that threadfin shad 138 and freshwater sardine 138 can purposely become a forage fish for more expensive farmed aquaculture such as salmon, trout, crappie, largemouth bass, hybrid striped bass, shrimp and red tail crawfish. Due to the prolific spawning rates of the threadfin shad 138 and freshwater, the present invention can enjoy surpluses of these two fingerlings for fishmeal.

The fishmeal model of the present invention uses a blend of naturally occurring blue green algae which are very high in omega-3 essential fatty acids, as well as the fish species that feed upon those omega-3 algae such as the fresh water threadfin shad 138 also known as Dorosoma petenense or the freshwater sardine (Freshwater Sardinella) 138 also known as Sardinella tawilis, either of which can be used quite effectively as high nutrient content fishmeal.

The fresh water shad 138 contains the highest level of omega-3 essential fatty acid of any fish meal fish in North America. This type of Sardine 138 is the only freshwater variation that is known to exist. The freshwater sardine 138 contains the highest level of omega-3 essential fatty acid of any fish meal fish in rest of the world's fresh water fisheries. Thereby an additional preferred method of this invention uses the included bioreactor 130 to incubate and grown plankton family organisms to provide biomaterial nutrients for food input for the reactor producing microorganisms for filter feeder fish 138 as a novel growth method such as when combined with the preferred method using this filter feeding threadfin shad 138 or the freshwater sardine 138 in amalgamation of adaptive biometrics and thermal imagining sensory analysis, monitoring and active control for increased system efficiency, production and high yields.

Either and/or both of these two fish are fed and grown via bioreactor 130 generated microalgae and organisms reactor production using adaptive biometrics and thermal imagining sensory analysis, monitoring and active control responses providing an active system of proactive and reactive health and growth system functions greatly improving species health and highest yields possible which by using this method gives the present invention a much improved advantage over prior art. The preferred embodiment exhibits an all-natural and completely organic food chain that is kept fully intact and fully realized. This method of fish meal production will provide all the nutrients, true proteins and omega 3 fatty acids that would be commonly found in the natural food chain. Thus, an aquaculture product 106 produced according to the present invention are higher quality products, higher quantity yields and contains higher nutritional value than any farmed aquaculture that is fed and/or raised on corn, soybean and other nitrogen rich crude protein based feed sources.

Preferred method of the present invention results in a premium quality consumer product that offers a natural and organic source of omega-3 essential fatty acids that is highly marketable to consumers, and its front-label placement on consumer packages is permitted under USDA and/or FDA Inbeling guidelines without any special USDA and/or FDA permits or reviews because it is a naturally occurring substance and an organically maintained process.

The present invention maintains its advantage by encapsulating this process which best emulates the natural food cycle while retaining ecological advantages by removing the entire cycle from potential contact or exposure to contamination of toxins and heavy metal commonly found in present day prior art aquaculture products 106 and aquatic species from in the wild, open and enclosed aquaculture settings 106. Additionally the preferred method prevents over fishing and heavy extraction of fish meal burden from the ocean, seas, lakes and waterways for the purpose and/or use in aquaculture 106.

Another advantage of the present invention is the preferred method of interchanging fresh water sardines 138 for the normal Krill and marine based Sardine food chain while retaining the aforementioned nutrient advantages versus prior art typical use of partially or wholly grain fed aquaculture. Salmon, trout, catfish and other aquaculture fed by this present inventions method and processes retain natural high quality food nutrient value to include healthy omega 3 fatty acids using the above enhanced method of fishmeal production.

Another advantage of the present invention is through improved use of isolated genetics enhancement through advancing favorable traits and elimination of unwanted traits of the aquaculture 106, aeroponics 104, algae and micro-organism by multi-generational selective breeding and crossbreeding through species specific adaptive biometric and thermal imaging analysis 604, monitoring and control. Further additional advantage can be achieved through use of selective gene amplification processes to enhance species grown within the aquaponics systems 100 for disease resistance, high reproduction rates, high growth rates, and high yields. The selective use of highly improved genetics can be very important to the viability and the ultimate success of an aquaponics system 100.

Other methods used to preserve fish and fish products include: (a) the control of temperature using ice, refrigeration or freezing; (b) the control of water activity by drying, salting, smoking or freeze-drying; (c) the physical control of microbial loads through microwave heating or ionizing irradiation; (d) the chemical control of microbial loads by adding acids; and (e) oxygen deprivation, such as vacuum packing or reduced oxygen content processing areas. Usually more than one of these methods is used conjunctively, further novel method of the present invention would include using adaptive biometrics, thermal imaging sensory and additional sensors for detection product contaminations, product quality assurance tracking 616 of all methods, applications and product, can be quickly and easily be identified. When chilled or frozen aquaculture or aquaculture products 106 are transported by pallet, forklift, conveyor belt. road, rail, sea or air, the cold chain must be maintained at all times. This requires insulated containers or transport vehicles and adequate refrigeration. Modern shipping containers can combine refrigeration with a controlled atmosphere. Aquaculture processing 134 is also concerned with proper waste management and with adding value to aquaculture products 106 and potential use for nutritional input for manufacturing of enriched pet foods.

By openly accepting public and/or municipal bio material waste, the initial startup and operating costs of importing biomass for a digest 112 are minimized. The digester unit 112 requires biodegradable materials to operate and provided the necessary CO2 generation, the material type of which can readily be found in any municipality and generally considered a nuisance for municipal garage dumps. The digestion unit is broadly designed to be commercially rigid unit that is capable of being situated on-site, scaled to the energy needs of each aquaculture 106 and agriculture production facility and openly accepting municipal waste systems as an input while generating at least CO2, liquid nutrients, nutrient rich solid waste as outputs of by-products such as biogas during its operation.

The preferred method of the present invention sets forth its primary advantage and novel method over prior art above applications and processes with physically connected preheaters and heating system elements, heat exchangers 129 and regenerators in its reclamation and recycling of waste thermal energy for use, reuse, storage and/or conversion and storage. This energy is used by thermal intensive applications such as with stirling cycle engines 126 which use a portion of the thermal energy for the generation of rotational energy, for use in such applications such as rotation work needed for input into a generator, pump or compressor. Waste heat recycled from this process may be used in a second level of reuse of available waste energy as thermal energy input into secondary lower heat threshold thermal intensive applications such as stirling engine cycle 126 with a reduced temperature differential which would then use a portion of the thermal energy input for the generation of rotational energy for us in such applications such as rotation work needed for input into a generator, pump or compressor.

The present invention has additional advantage over prior art from additional applications and process cycles from remaining thermal energy and from storage to further encourage use and recycling of available energy for additional application and processes energy usage that may be added based on available input temperatures and return on investment cost versus an acceptable benefits to costs ratio, all remaining recyclable thermal energy may then be reclaimed and then communicated to appropriate temperature thermal storage systems, additionally thermal energy may be communicated to absorption cooling 124 to convert heat based thermal energy into cold based thermal energy to maintain a localized energy balance of available stored thermal energy. The preferred method of the present invention sets forth its primary advantage and novel method over prior art provides for normalized thermal energy balance that is essential for realized and optimized system wide use and reuse efficiencies concurrently monetizing all energy input for all intended applications and processes.

Prior art of directed-energy applications and processes typically was never fully or partially automated due to its inherent design and deployment flaws. The preferred method of the present invention uses metrics, biometrics and thermal imaging technologies of analysis, monitoring and control of the directed-energy process using amalgamated with artificial intelligence 624 and automation including robotics to reduce or eliminate injuries and enhanced uptime, productivity and enhanced volume production.

Prior Art generally used energy input in the form of grid energy supplied or mostly provided by grid with its inherent cost and price escalation. Embodiments of the invention will employ renewable energy as the primary electrical and thermal energy input for the purpose of electrical energy generation, thermal applications and energy storage 122. The preferred method of the present invention communicates thermal energy from thermal storage for the purpose of providing thermal energy for preheating, heating and recycling thermal energy from the energy processes.

Embodiments of the invention will introduce and extend artificial intelligence 624 interfaced component layers, layers will include but not limited to building, robotics, applications and device's automation system, utilizing hardware and software based monitoring, analysis and control system for enhanced performance, efficiencies, power quality analysis 706, energy cost tracking, energy demand control 708, energy efficiency automation. Additional layers include inventory monitoring, accounting, analysis 603, reporting 618 and control.

The central energy embodiment encompasses an intelligent interface interconnecting monitor, analysis and control elements to improve reliability, manage process flows, enabling increased commercial yields, cost reduction and reduced loss of production and service availability. Maximizing infrastructure utilization to achieve lowest possible levelized cost of energy is achieved by monetizing capital intensive fixed assets while reducing overlap and needless redundant processes.

This monitoring and analysis can be through sensors for local and remote purposes and may include video and thermal based sensors 103 input for uses such as adaptive biometrics and thermal imaging for monitoring, analysis and control. This may be combined into a species by species incorporating usage of metrics, biometric and thermal imaging sensors 604, monitoring, analysis and control regime and may include other environmental input and control as well as involvement into the full grow cycles including germination, planting and/or placement, grow out and harvest 410. Similar process would be used in aquaponics 100 with inclusion in cycles such as hatchery, fingerlings, grow out and harvesting 410.

The current invention produces substantially reduced investment capital requirements, encapsulated by capturing enhanced value on capital expenditures with greatly increased return on investments. Embodiments when paired with its energy storage 122 and waste heat recovery system using reclaimed energy, system is able to actualize and realize the maximum benefits and utilization of all available system resources.

Prior Art smartgrid designs and integrations primarily use smart meters on consumer connections to monitor usage. Improving upon previous art of smartgrid implementation of the current invention is effectuated via monitoring usage, identifying the energy usage sources through device data transmitting, manual consumer input and from its common electrical signal fingerprint, storing profile data sets, responding with appropriate energy assumptions from extracted usage profiles, analysis of time of day usage for enhanced energy load response for power quality 706 and energy availability to enhance grid stability.

The electronic monitoring, identification, energy generation, base-load energy load response and energy provisioning to satisfy grid stability from supply compensation for end use requirements and control element of the present invention in the current application shall henceforth be known and designated from the above as elements for the features and functionality as system to be known as “ULTRAGRID™”700.

An enhanced approach to commercial grid energy storage 122 is inclusion of Ultragrid 700. The current power grid is designed and developed unable to allow generation sources to respond to on-demand to consumer needs, while an Ultragrid 700 based smart grid can be designed so that usage varies on-demand with production availability from intermittent power sources such as wind and solar and stabilized by matched stored energy release for commercial grid generation for both electrical and/or thermal intensive systems. End-user loads can be proactively projected and timed for a concerted startup during peak usage periods or the cost of energy can dynamically vary between peak and nonpeak periods to encourage turning off non-essential high energy loads or control application startup to not occur simultaneously.

The present invention with its elements for the features and functionality as system to be known as Modular Advanced Intelligent Commercial Energy System (MAICES) forms a foundation and basis for distributed electrical, chemical and thermal energy, localized storage reserves preserving electrical, chemical, thermal energy and supply security. The present invention provides storage reserves of electrical, chemical, thermal energy availability during natural and manmade catastrophic accidents to energy and fuel supplies.

This invention is directed towards providing for a hybrid aquaponics system 100 which will solve or at least minimize some of the problems with conventional methods and systems. The preferred invention uses a tank based system of aquaculture 106 that are grown out in tanks and are fed nutrient complete diets. Fresh water is continuously pumped to pass through the tank systems to remove waste and to maintain suitable nontoxic growing conditions. Yields based on the area of tank system, can be up to 9,500 to 12,000 tons per hectare, however, this is based on water requirements for example (265 gallons per minute, per ton of a certain fish species).

The present invention relates to an aquaponics system 100. This invention has particular application to farming systems for combined breeding, grow out 204 and harvesting 206 of aquatic species and growth of vegetables, and for illustrative purposes the invention will be described hereinafter with reference to this application. However, it can be easily appreciated that this invention may find use in alternate applications, such as breeding, grow out 204 and harvesting 206 of crustaceans or other specialized aquatic species and/or growth of any other suitable plant species.

The present invention provides an aquaponics system 100 including: a tank for housing at least one aquatic animal species; a plant growing apparatus for housing one or more plant species growing in an aqueous environment; and biofilter module 108 for receiving a waste stream including waste and water from each of the tank and the aeroponics system plant growing apparatus 104, the biofilter module 108 including: a large solids removal means; and a biological waste digestion unit for digesting solids to produce plant nutrients; wherein said biological waste digestion unit includes a biological species that at least partially digests waste from said aquatic species to plant nutrients; whereby in use, said plant nutrients are transferred to the aeroponics system 104 and plant growing apparatus with at least some of the potable water is returned to the tank.

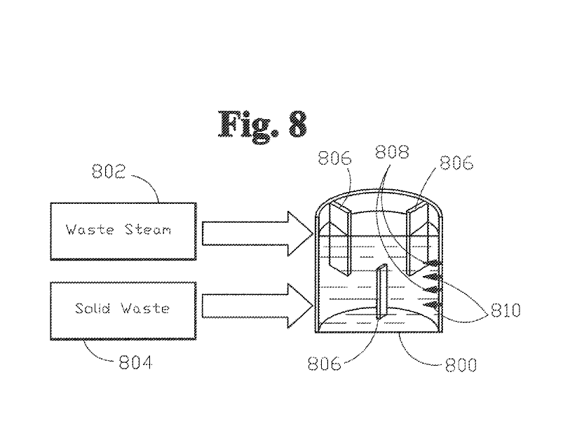

Referring now to FIG. 8, a diagram of the nitrification entity 800 in accordance with the preferred embodiment of the system 100 is shown. The preferred embodiment includes a nitrification means for treating waste water streams 802 and/or solid waste 804. This nitrification means may include any nitrifying entity 800 capable of nitrifying ammonia, for example it may include known methods of prior art consisting of appropriate chemical, a zeolite filter or any nitrifying microorganism. In a preferred embodiment the nitrification means may include one or more species of nitrifying bacteria, for example Nitrosomonas and nitrobacterium. Preferably the nitrification means may also include a high surface area medium, for example bio-balls. The nitrification means may include a tank for housing said nitrification entity 800, wherein said tank is separate fro the plant growing apparatus. The nitrification tank may include one or more baffles 806 to aid in directional flow of water within the tank as well as air jets 808 and water jets 810 as well.

A primary purpose of the invention is to advance the art of aeroponics 104 in amalgamation with adaptive biometrics, thermal imaging sensory and artificial intelligence 624 using controls and adaption of environmental settings to include a high level of input control of photo-sensitive biochemical activity in plants, particularly photomorphogenesis and photosynthesis, this should not to be construed as a limitation of other potential benefits. The preferred method of the present invention is the ability to use artificial intelligence 624 for monitoring, analysis and control through programmable and controlled emissions of phototropically active portions of the light spectrum though both amplitude and time domain modulation in conjunction of nutrient loading and moisture control which shall be then synchronized and harmonized with related metabolic and growth cycle processes to stimulate and control the intended botanical species.

The invention comprises an apparatus and method for plant metabolism manipulation using the spectral output of light sources such as LEDs or other available prior art. The use of an artificially enhanced digitally controlled source of light (such as an LED) rather than high intensity discharge lamps (HID) offers further economic advantages with its low power consumption, low heat generation during operation, and vastly improved life spans common in the LED lighting industry. Further advantages of the present invention through the special properties of LED such as specific spectrum wavelength colors and amplitude and more specifically through the ability of near infinite control of the various exposure time to specific amplitude levels of energy at those specific spectrum wavelengths.

The light sources can be configured in amalgamation of desired wavelengths to suit specific plant photosynthesis and other phototrophic metabolic functions needs during propagation 406, vegetation and the fruiting/flowering stage independently of each individually targeted species. Alternatively the light source emitters can be configured to inhibit plant growth of unwanted plants as well as other industrial applications such as curing paint or adhesives. Also, the emitters can be oriented in any direction and in close proximity to the plants without damaging the leaves with excessive waste heat contact, or requiring more cooling energy input into the aeroponics growth area 408. The light source of this invention is operated by a progressive artificial intelligence and machine learning based computer controlled system 624 that uses adaptive biometrics and thermal imaging sensory to detect, analyze and control plant growth cycles, maximize metabolism efficiency, reducing product loss and deliver higher yields. As such, it represents a very significant step forward in the state of the art aeroponics 104 and the use of artificial light sources.

Prior art artificial light sources are not capable of accurately simulating the type of light in frequency and amplitude a plant would receive at dawn (pre-glow) or at dusk (after-glow) as the sun rises and sets. The preferred embodiment of the previous invention offers the ability to simulate this type of light source control has a positive effect on plant growth and improves the ability to manipulate a specific plant species metabolism to maximize plant growth and nutrient and water input requirements through adaptive biometric and thermal imaging analysis 604, monitoring and control, this has additional advantages of computer controlled spectrum emissions use of adaptive artificial intelligence 624 control of our invention which then includes the ability to force flowering, manipulate inter-nodal distances, eliminate vegetative regression, and initiate and derive root propagation of each species independently.

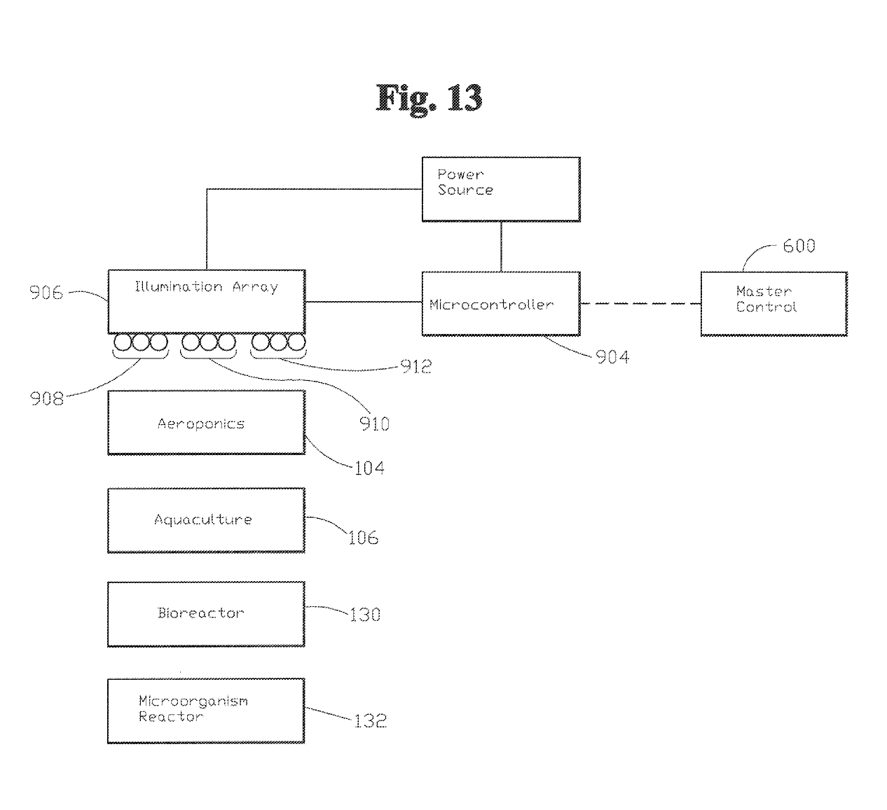

Referring now to FIG. 13, the system 100 preferably includes a control system 600 for controlling the illumination system 906, heating system, nutrient mix supply system, and, where provided, the climate and environmental control system 610—control of the illumination system 906 is done through a connected microcontroller 904 as depicted. To ensure optimum growing conditions within the chamber, the microcontroller 904 may include a computer and/or a programmable logic controller and/or networked device interface connected to said control system 600. Preferably the illumination means comprises a plurality of HDI and/or LEDs and/or other past art illumination devices. The preferred embodiment of the present invention includes at least one first plurality 908, second plurality 910, and third plurality 912 light sources.

An array or multiple arrays of energy efficient HDI and/or LEDs lighting are configured in proximity to plant life to emit light energy in a variety of photosynthetic promoting frequencies and power outputs. Specific light frequencies are selected after analyzing the photosynthetic properties of the selected plant of interest. The HDI and/or LEDs lighting are locally monitored, analyzed and controlled or remotely monitored, analyzed and controlled via adaptive biometrics, thermal imaging sensory and additional sensors with an environmental control system 610 and artificial intelligence machine learning system 624 that can be remotely monitored, analyzed and controlled through a handheld device using a GUI adapted for that purpose. A variety of HDI and/or LEDs lights emitting a variety of photo synthetically useful light wavelengths are arrayed together while independently controlled.

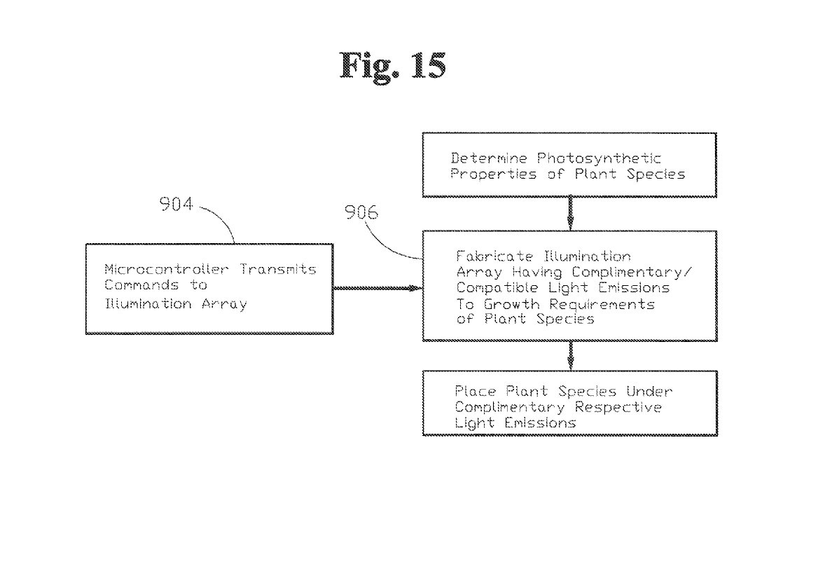

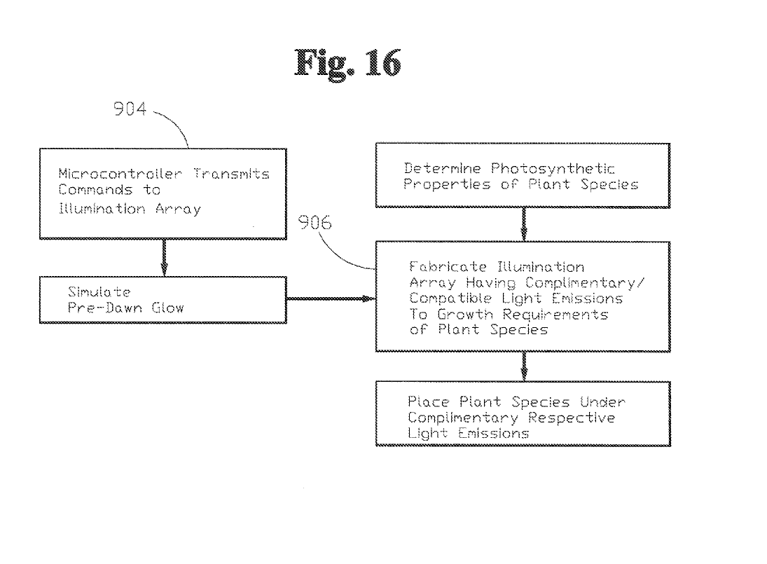

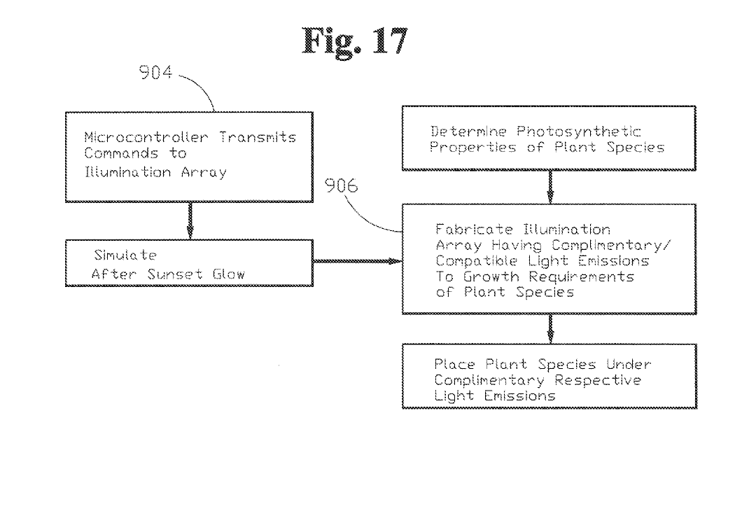

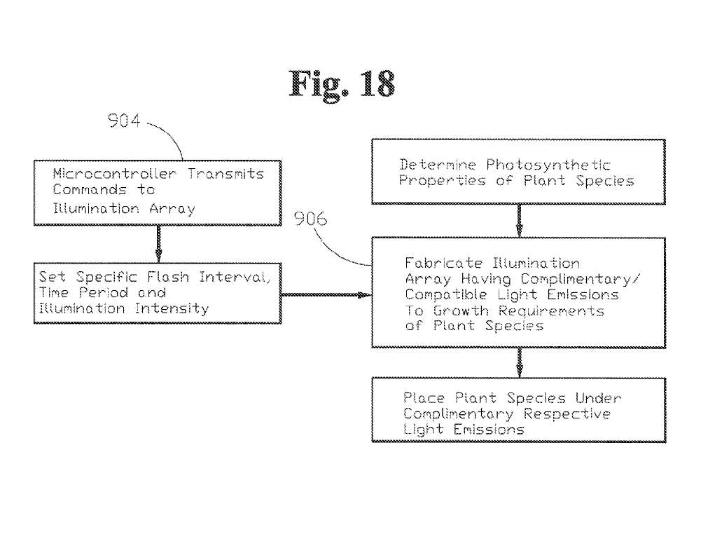

Referring now to FIGS. 15-18, flowcharts depicting methods for manipulation of plant metabolism using spectral output are shown. The method allows the system 100 of the present invention to determine compatible light emissions for a target plant species. The system 100 may then utilize an illumination array 906 having one or more plurality of light sources 908 that compliments said targeted plant species. The array 906 can be controlled by a microcontroller 904 to emit pre-dawn and after-sunset glows or predefined harvest cycles. The lighting array 906 can also be used to inhibit plant growth by adjusting light availability for time of day and through adjustment of light wavelength frequencies via the microcontroller 904. Additionally, the microcontroller 904 of the preferred embodiment integrates with a receives commands from said control unit 600 for the system 100.

An additional aspect the present invention provides an aquaponics system 100 a method for symbiotic rearing of one or more aquatic species and one or more plant species including:

a) providing:
i) a tank which houses one or more aquatic species;
ii) an aeroponics plant growing apparatus 104 which houses one or more plant species growing in an aqueous environment; and
iii) a biofilter module 108 for receiving a waste stream including solid waste and water from the tank, the biofilter module 108 comprising a solids removal means and a biological waste digestion unit for digesting solids from the solids removal means to produce plant nutrients, which biological waste digestion unit comprises a biological species that at least partially digests solid waste from said solids removal means to plant nutrients;

b) transferring solid waste from said tank to said biofilter module 108; and

c) transferring said plant nutrients from said biofilter module 108 to said plant growing apparatus.

An additional aspect of the present invention also provides an aquaponics system 100 a method for symbiotic rearing of one or more aquatic species and one or more plant species. Preferably the method includes:

a) providing:
i) a tank for housing at least one aquatic species;
ii) an aeroponics plant growing apparatus 104 for housing one or more plant species growing in an aqueous environment; and
iii) a biofilter module 108 for receiving a waste stream including waste and water from each of the tank and the aeroponics plant growing apparatus 104, the biofilter module 108 including; large or heavy solids removal means; and a biological waste digestion unit for digesting solids to product plant nutrients; wherein said biological waste digestion unit includes a biological species that at least partially digests waste from said aquatic species to plant nutrients;

b) housing said aquatic animal species in said tank and housing said plant species in said aeroponics plant growing apparatus 104;

c) transferring water and waste from said tank to said biofilter module 108;

d) transferring plant nutrients and a portion of the water exiting said biofilter module 108 to said aeroponics plant growing apparatus 104; and

e) returning at least a portion of said water to storage.

The design aspects of the current invention may allow the provision of at least a partially closed circuit aquaponics system 100. In the preferred embodiment the invention provides a closed circuit system. A closed circuit aquaponics system 100 is one in which the entire environmental cycle of the wastes produced by the biological species in the system are recycled through the system with very little to no expulsion of waste (including aquatic species excrement and plant matter). A partially closed circuit aquaponics system 100 is one in which expulsion of waste is minimal, with the majority of waste being recycled and reused to enhance efficiency through the entire system.

The use of a biological waste digestion unit independent of the plant growing apparatus allows control of input oxygen into digesters 112 to enhance performance while using waste gases to supply of CO2 back into the aeroponics system 104 for enhanced plant growth while both water and degraded waste to be recycled within the system without the need to continuously expel water and effluent from the system after it has passed through components of the system, whilst requiring reduced or even no plant media. This is obviously advantageous from a conservation perspective and may facilitate the potential use of aquaponics system 100 in environments where they may not normally be suitable, for example in urban settings.

Also the use of an aquaponics system 100 in which minimal input of water is required, for example only such water that is required to replace evaporation from the system needs to be added to the system, is obviously advantageous in times of water shortage. Furthermore, the system may be more productive because higher levels of nutrients are retained within the system that can be used for increased plant growth. Regular input of food for the aquatic species may be required and similarly occasional cleaning (including expulsion) and replenishment of other inputs such as water may be required. Embodiments are envisaged where the primary food source for the aquatic species is also integrated into the system.

The tank containing the aquatic species may be any appropriate shape. In a preferred embodiment the tank may be designed to allow reversible unidirectional circular flow throughout the tank, for example the tank may include a baffle through the tank. Additionally the tank may stackable to increase density per acre and may include air or water jets to propel water in a particular direction. the preferred embodiment the aquatic species may be an appropriate species, for example any species of fish, crustaceans, shellfish or mollusks.

The solids removal means may be any appropriate means for separating particulate matter from water or particles of a predetermined minimum size (typically large particles such as particles of 50 microns or more) from smaller particles and water (the latter being termed a large solids removal means). In a preferred embodiment the solids removal means includes a filter 109, such as a drum filter. The filter 109 may be appropriately sized depending on the aquatic species housed in said tank. In a preferred embodiment the filter 109 allows delivery of the solids stream to the biological waste digestion unit with minimal water content. In another embodiment, the solids removal means includes a swirl separator, which is a conical chamber which passively settles the heavier solids in the waste stream. The overflow to the swirl separator may, for example, be directed to another solids removal means, such as a filter as described above, for the removal of solid matter that has not been captured by the swirl separator.

It is also possible for the solids removal means to include more than one system for separation/removal of solids (or alternatively expressed, the system may comprise more than one solids removal means). For example, the solid removal means can include both a swirl separator and a filter 109, such as a drum filter. In a particular embodiment, the different systems are supplied in parallel from the tank e.g. a swirl separator is supplied with a waste stream from the bottom, or a lower portion, or the tank, typically gravity fed. A filter 109 is supplied with a waste stream from a stand pipe, or similar, at the surface of the water in the tank.

The system may also comprise a foam fractionator or ‘protein skimmer’ connected to the output of a nitrification system 800. Waste water upon leaving the nitrification system 800 may be processed via a foam fractionator or ‘protein skimmer’ whereby fine yet suspended particles and dissolved proteins are able to be removed from the water as surface foam. This waste stream that is in the form of foam is then delivered to the next phase of handling which is the biological waste digestion unit.

The solid waste, such as a rich sludge, collected by the one or more components of the solid removal means is transferred to a biological waste digestion unit. In the case of filters, such as drum filters, the collected solid waste is typically periodically back-washed by water in the system, for example using water delivered onto the filter mesh or screen by pressurized liquid stream jets. In the case of a swirl separator, the solid waste can be transferred out to drain the separator, allowing solids to drop into the biological waste digestion unit by gravity and/or with the assistance of some additional system water. Other suitable solids removal means and methods for transferring collected solids to the biological waste digestion unit will be readily apparent to a person skilled in the art.

In a preferred embodiment the biological species in the biological waste digestion unit is a species of worm. In a preferred embodiment the biological waste digestion unit is a worm unit. Other suitable biological species include insect larvae, as are described in more detail below. The role of the biological species in the biological waste digestion unit, such as compost worms, the simplistic terms, is to convert solid wastes from the aquaculture tank into a form more suitable (e.g. worm castings) for reintroduction into the system. Uneaten aquaculture feed and feces, if not thoroughly processed, are a potential source of disease. Once passed through the worm's gut however the castings can safely be reintroduced as a liquid plant food that also supports a colony of microorganisms carrying out other important functions such as buffering, nutrient cycling and disease repression.

The plant growing apparatus may be any suitable apparatus that allows the growth of plants in an aquatic environment. For example the apparatus may contain hollow tubes through which water an nutrients exiting the biofilter module 108 may be passed, with upper opening for entry of plant roots. The plant growing apparatus my be a stacked apparatus including multiple layers of plant troughs. For example, the apparatus may take the form of a multiple level A-frame or ladder-type structure.

With water exiting the plant growing apparatus will generally exhibit some solid matter including plant debris and growth media (potentially only if used), this water is typically passed through, or transferred to, a filter 109 to separate the solid matter from the water prior to the water being directed to the tank. In preferred embodiment water exiting the plant growing apparatus is directed back to the solid removal means, such as to a filter/drum filter component 109 of the solid removal means.

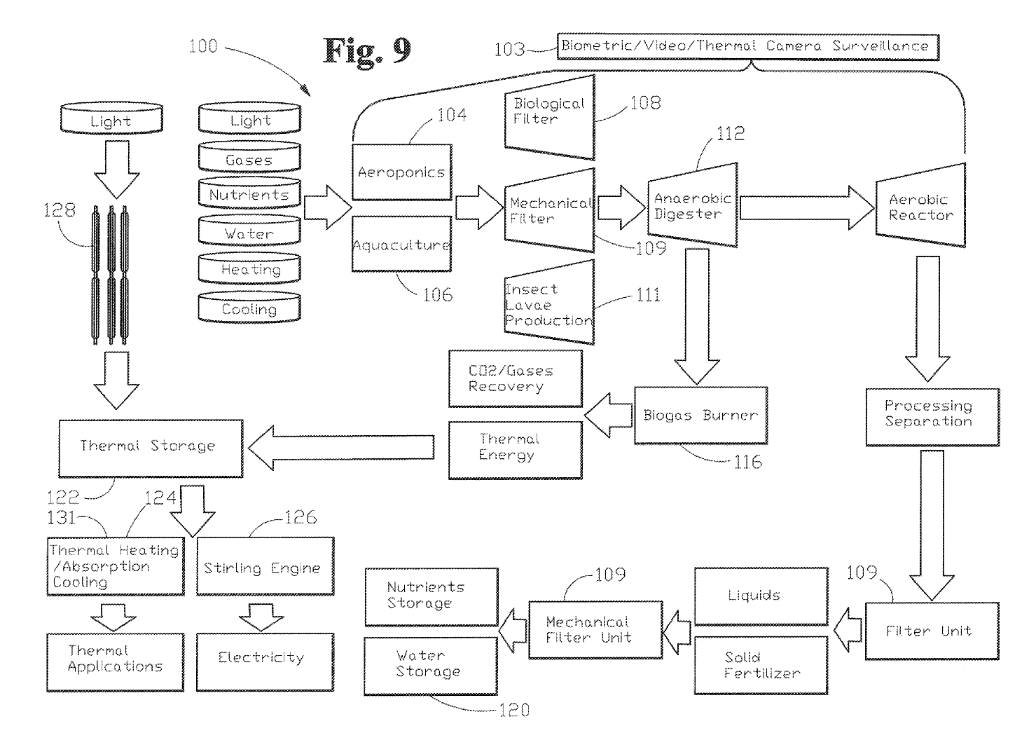

As depicted in FIG. 9, the system 100 may optionally include an insect larvae production module 111. Said module would include a reversibly sealable container for housing organic waste and suitable insect species, an insect larvae outlet pipe to direct larvae from said container and optionally an insect larvae collection means. The aforementioned alternative embodiment would allow increased efficiencies of the filter units 1009 by utilizing the insect population to break down waste collected in said filter units 109.

The size of the components of the current system will be generally co-dependent, i.e. the size of the tank will directly affect the number of aquatic organisms which may be maintained, which will in turn directly affect the amount of nutrients produced via the biological waste digestion unit, which in turn directly will affect the amount of plants that may be grown. In a preferred embodiment the plant growing apparatus may be able to grow sufficient plants per square foot to take up the available nutrients from the aquatic species maintained in the tank in the same surface area as the tank and biological waste digestion unit combined.

In one embodiment the current system is designed to be used in urban farming environments where space is a premium. To facilitate such an embodiment the current system has the benefit of being able to be scaled appropriately depending on the requirements. For example in an urban setting the components of the system would be stacked where appropriate, e.g. the aeroponics 104 or aquaculture growing apparatus 106 may be a suitable for vertical stacking of growing apparatus by a horizontal orientated. Additionally, the individual components of the system may be vertically stacked horizontal orientation in an appropriate stacked order. Other systems and/or functions may be vertically stacked below the system

In embodiments where the system may be vertically stacked the system has potential use in a number of settings in which it would not previously been suitable for an aquaponics system 100 to be established. For example, the stacking may allow the use of the system in urban settings and densely populated areas where horizontal space is restricted. Additionally the partially closed nature of the system, as described above, may also facilitate its use in such settings as there is no issue with transfer or disposal of waste or water.

Some closed or partially closed circuit aquaponics systems do exist, however such systems typically house any waste converting components within the media held in the plant beds. These beds typically contain worms and/or other waste-converting organisms, which turn solid waste into ‘plant nutrients’ which can then be taken up by plants growing in the clay beads and/or clay balls and/or gravel beds medium. In such systems typically all water and solid wastes are passed through the plant media beds. Limitations such as this and other prior art deficiencies have limitations to which is the aims of the present invention. Cleaning and maintenance of the system is difficult as the plant media need to be removed from the replaced into the plant beds regularly as waste builds up. This is quite tedious and labor intensive. Furthermore, the raw waste from the tank is held in the media through which the full flow of water continually passes. The system of the current invention is designed such that the solid wastes are quickly isolated such that only a small percentage of water passes through untreated waste. The current system is thus able to maintain a much greater aquaculture density without fear of biological problems or collapse. Consequently, the higher fish density results, in more concentrated nutrients within the system and increased potential for plant growth.

It should be understood that the preferred embodiment of the present invention primarily comprises five interdependent biological systems, the aquaculture in the aquaculture tanks, the filter feeder aquaculture, the bacterium in the bioreactor 130, the microorganisms in the biofilter 108, and the plants in the aeroponics facility 104. The relationships between these biological systems are dynamic, and a proper balance should be maintained between these systems for optimum functioning of each. When all five of the biological systems are operating efficiently, the combined aquaculture facility 106 and aeroponics facility 104 will offer a great many advantages and few if any drawbacks.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g. attached, adhered, joined) are to be construed broadly and may include intermediate members between a connection of elements and relative movement between elements. As such, joinder references do not necessarily infer that two elements are directly connected and in fixed relation to each other. Moreover, network connection references are to be construed broadly and may include intermediate members or devices between network connections of elements. As such, network connection references do not necessarily infer that two elements are in direct communication with each other. In some instances, in methodologies directly or indirectly set forth herein, various steps and operations are described in one possible order of operation, but those skilled in the art will recognize that steps and operations may be rearranged, replaced or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements and/or substantial equivalents.

What is claimed is:

1. An aquaponics system comprising at least one aquaculture unit, at least one aeroponics unit, and at least one subunit utilizing at least one of a common nutrient and waste stream; wherein said at least one subunit is selected from the group consisting of:
a. At least one microalgae bioreactor unit;
b. At least one microorganism reactor unit;
c. At least one filter feeder unit;
d. At least one filter unit;
e. At least one digester unit; and
f. Combinations thereof.

2. The aquaponics system of claim 1 further comprising at least one of a camera unit and thermal camera unit as well as at least one control and wherein said at least one control unit comprises a computer having a first processor and a first machine readable medium storing instructions providing at least one of artificial intelligence, machine learning system, computer interfaced adaptive metrics, biometrics and thermal analysis system in addition to facility control instructions connected through a network to execute commands automatically or through manual intervention by a user.

3. The aquaponics system of claim 1 wherein said at least one digester unit comprises at least one anaerobic digester and aerobic digester.

4. The aquaponics system of claim 1 further comprising a renewable power unit wherein said renewable power unit comprises at least one thermal solar panels. Stirling engine, and thermal storage facilities.

5. The aquaponics system of claim 4 further comprising:
a. At least one atmospherically sealed processing unit;
b. At least one atmospherically sealed packaging unit:
e. At least one atmospherically sealed dry storage unit; and
d. At least one atmospherically sealed cold storage unit.

6. Use aquaponics system of claim 5 wherein refrigeration for said cold storage unit and heating and cooling for said aquaponics system is provided by said thermal storage facilities and an absorption cooling system with hot and cold thermal storage attached therein.

7. The aquaponics system of claim 1 where said system provides:
a. At least one aquaculture tank containing at least one aquatic animal species;
b. At least one aeroponics unit containing at least one plant species;
c. At least one microalgae bioreactor containing at least one microalgae species;
d. At least one organism reactor containing at least one microorganism; and
e. At least one filter unit for receiving a waste stream comprising solid waste and waste water from said aquaculture tank.

8. The aquaponics system of claim 7 wherein said at least one filter unit further comprises at least one solid waste removal means.

9. The aquaponics system of claim 1 wherein said at least one aeroponics unit comprises:
a. At least one germination area;
b. At least one grow area;
c. At least one water and nutrient storage area; and
d. At least one area for horticulture finishing;

10. The aquaponics system of claim 1 wherein said at least one aquaculture unit comprises:
a. At least one area for pisciculture;
b. At least one area for grow out;
c. At least one pre-processing area;
d. At least one processing area;
e. At least one preparation area;
f. At least one chilling area; and
g. At least one storage area.

11. The aquaponics system of claim 1 wherein said filter unit comprises:
a. At least one mechanical filter;
b. At least one biological filter;
c. At least one anaerobic digester; and
d. At least one aerobic digester.

12. The aquaponics system of claim 2 wherein said at least one control unit provides control of at least one of illumination, heating, cooling, humidity, nutrient mix, gas composition and nutrient supply.

13. The aquaponics system of claim 12 wherein water collected by at least one of heating, cooling and humidity control means and at least one said digester is used by said nutrient supply.

14. The aquaponics system of claim 2 wherein said at least one control unit analyzes, identifies, monitors, tracks and records at least one plant species, animal species and microorganism through said at least one of a biometric camera unit and thermal camera unit.

15. The aquaponics system of claim 14 wherein said automation provided by at least one control unit provides improved genetics of aquatic species, plants microorganisms within said aquaponics system.

16. The aquaponics system of claim 14 wherein said at least one control unit provides automated, customized and individualized feeds, nutrients, and supplements to said at least one plant species, animal species and microorganism and reduce or eliminate unwanted insects and vermin in said aquaponics system.

17. The aquaponics system of claim 2 wherein said at least one control unit and said at least one of a biometric camera unit and thermal camera unit provide identification, monitoring, tracking, and record keeping of individuals within said aquaponics system for security purposes.

18. The aquaponics system of claim 2 wherein said at least one control unit communicates with and controls at least one robotic apparatus.

19. The aquaponics system of claim 18 wherein said at least one robotic apparatus provides said at least one control unit with automation, harvesting and processing capabilities to increase the efficiency of said aquaponics system.

20. The aquaponics system of claim 12 wherein said at least one robotic apparatus provides:
a. Transfer of seeds to said at least one germination unit;
b. Transfer of seeds to said at least one grow out unit;
c. Unmanned harvesting and pre-processing of said harvest; and
d. Transfer of said pre-processed harvest to a packaging and storage unit.

21. The aquaponics system of claim 1 wherein said system is a partially closed circuit.

22. The aquaponics system of claim 8 wherein said system is a closed circuit.

23. The aquaponics system comprising:
a. At least one atmospherically sealed aeroponics unit;
b. At least one atmospherically sealed aquaculture unit;
c. At least one atmospherically sealed microalgae bioreactor unit;
d. At least one atmospherically sealed microorganism reactor unit;
e. At least one of a biometric camera unit and thermal camera unit; and
f. At least one control unit wherein said at least one control unit comprises a computer having a first processor and a first machine readable medium storing instructions providing at least one of artificial intelligence, machine learning system, computer interfaced adaptive metrics, biometrics and thermal analysis system in addition to facility control instructions connected through a network to execute commands automatically from said artificial intelligence or through manual intervention by a user.

24. The aquaponics system of claim 23 further comprising:
a. At least one atmospherically sealed filter feeder unit;
b. At least one atmospherically sealed filter unit;
c. At least one atmospherically sealed digester unit;

25. The aquaponics system of claim 24 wherein said at least one digester unit comprises at least one anaerobic digester and aerobic digester.

26. The aquaponics system of claim 23 further comprising a renewable power unit wherein said renewable power unit comprises at least one thermal solar panels, Stirling engine, and thermal storage facilities.

27. The aquaponics system of claim 26 further comprising:
a. At least one atmospherically sealed processing unit;
b. At least one atmospherically sealed packaging unit;
c. At least one atmospherically sealed dry storage unit; and
d. At least one atmospherically sealed cold storage unit.

28. The aquaponics system of claim 27 wherein refrigeration for said cold storage unit and heating and cooling for said aquaponics system is provided by said thermal storage facilities and an absorption cooling system with hot and cold thermal storage attached therein.

29. The aquaponics system of claim 24 where said system provides:
a. At least one aquaculture tank containing at least one aquatic animal species;
b. At least one aeroponics unit containing at least one plant species;
c. At least one microalgae bioreactor containing at least one microalgae species;
d. At least one organism reactor containing at least one microorganism; and
e. At least one filter unit for receiving a waste stream comprising solid waste and waste water from said aquaculture tank.

30. The aquaponics system of claim 29 wherein said at least one filter unit further comprises at least one solid waste removal means.

31. The aquaponics system of claim 23 wherein said at least one aeroponics unit comprises:
a. At least one germination area;
b. At least one grow area;
c. At least one water and nutrient storage area; and
d. At least one area for horticulture finishing;

32. The aquaponics system of claim 23 wherein said at least one aquaculture unit comprises:
a. At least one area for pisciculture;
b. At least one area for grow out;
c. At least one pre-processing area;
d. At least one processing area;
e. At least one preparation area;
f. At least one chilling area; and
g. At least one storage area.

33. The aquaponics system of claim 24 wherein said filter unit comprises:
a. At least one mechanical filter;
b. At least one biological filter;
c. At least one anaerobic digester; and
d. At least one aerobic digester.

34. The aquaponics system of claim 23 wherein said at least one control unit provides control of at least one of illumination, heating, cooling, humidity, nutrient mix, gas composition and nutrient supply.

35. The aquaponics system of claim 35 wherein water collected by at least one of heating, cooling and humidity control means and at least one said digester is used by said nutrient supply.

36. The aquaponics system of claim 23 wherein said at least one control unit analyzes, identifies, monitors, tracks and records at least one plant species, animal species and microorganism through said at least one of a biometric camera unit and thermal camera unit.

37. The aquaponics system of claim 36 wherein said automation provided by at least one control unit provides improved genetics of aquatic species, plants microorganisms within said aquaponics system.

38. The aquaponics system of claim 36 wherein said at least one control unit provides automated, customized and individualized feeds, nutrients, and supplements to said at least one plant species, animal species and microorganism and reduce or eliminate unwanted insects and vermin in said aquaponics system.

39. The aquaponics system of claim 23 wherein said at least one control unit and said at least one of a biometric camera unit and thermal camera unit provide identification, monitoring, tracking, and record keeping of individuals within said aquaponics system for security purposes.

40. The aquaponics system of claim 23 wherein said at least one control unit communicates with and controls at least one robotic apparatus.

41. The aquaponics system of claim 40 wherein said at least one robotic apparatus provides said at least one control unit with automation, harvesting and processing capabilities to increase the efficiency of said aquaponics system.

42. The aquaponics system of claim 40 wherein said at least one robotic apparatus provides:
a. Transfer of seeds to said at least one germination unit;
b. Transfer of seeds to said at least one grow out unit;
c. Unmanned harvesting and pre-processing of said harvest; and
d. Transfer of said pre-processed harvest to a packaging and storage unit.

43. The aquaponics system of claim 23 wherein said system is a partially closed circuit.

44. The aquaponics system of claim 23 wherein said system is a closed circuit.

45. A method for operating an aquaponics system water supply comprising:
a. Providing water to at least one aquaculture unit from at least one water storage unit;
b. Providing water from at least one aquaculture unit to at least one aeroponics unit;
c. Said water passing through at least one of a biofilter, filter feeder, and digester before reaching said aeroponics unit;
d. Collecting atmospheric water from plant transpiration in said at least one aeroponics unit; and
e. Returning said collected water to said at least one water storage unit.

46. An aquaponics system comprising:
a. At least one atmospherically sealed aeroponics unit containing at least one plant species;
b. At least one atmospherically sealed aquaculture unit comprising:
i. At least one aquaculture tank containing at least one aquatic animal species;
c. At least one atmospherically sealed microalgae bioreactor containing at least one microalgae species;
d. At least one atmospherically sealed microorganism reactor containing at lest one microorganism;
e. At least one atmospherically sealed filter feeder unit;
f. At least one atmospherically sealed filter unit for receiving a waste stream comprising solid waste and waste water from at least one of said at least one aquaculture tank and at least one said aeroponics unit;
g. At least one atmospherically sealed digester unit;
h. At least one biometrics camera unit;
i. At least one thermal camera unit;
j. At least one atmospherically sealed processing unit;
k. At least one atmospherically sealed packaging unit;
l. At least one atmospherically sealed dry storage unit;
m. At least one atmospherically sealed cold storage unit; and
n. At least one control unit wherein said at least one control unit comprises a computer having a first processor and first machine readable medium storing instructions providing at lest one of artificial intelligence, machine learning system, computer interfaced adaptive metrics, biometrics and thermal analysis system in addition to facility control instructions connected through a network to execute commands automatically from said artificial intelligence or through manual intervention by a user.

47. The aquaponics system of claim 46 where said at least one digester unit comprises at least one anaerobic digester and aerobic digester.

48. The aquaponics system of claim 46 further comprising a renewable power unit wherein said renewable power unit comprises at least one thermal solar panels, Stirling engine, and thermal storage facilities.

49. The aquaponics system of claim 48 wherein refrigeration for said cold storage unit and heating and cooling for said aquaponics system is provided by said thermal storage facilities and an absorption cooling system with hot and cold thermal storage attached therein.

50. The aquaponics system of claim 46 wherein:
a. said at least one aeroponics unit comprises:
i. At least one germination area;
ii. At least one grow area;
iii. At least one water and nutrient storage area; and
iv. At least one area for horticulture finishing;
b. said at least one aquaculture unit comprises:
i. At least one area for pisciculture;
ii. At least one area for grow out;
iii. At least one pre-processing area;
iv. At least one processing area;
v. At least one preparation area;
vi. At least one chilling area; and
vii. At least one storage area.
said filter unit comprises:
i. At least one mechanical filter;
ii. At least one biological filter;
iii. At least one anaerobic digester; and
iv. At least one aerobic digester.

51. The aquaponics system of claim 46 wherein said at least one control unit provides control of at least one of illumination, heating, cooling, humidity, nutrient mix, gas composition and nutrient supply.

52. The aquaponics system of claim 51 wherein water collected by at least one of heating, cooling and humidity control means and at least one said digester is used by said nutrient supply.

53. The aquaponics system of claim 46 wherein said at least one control unit analyzes, identifies, monitors, tracks and records at least one plant species, animal species and microorganism through said at least one of a biometric camera unit and said at least one thermal camera unit.

54. The aquaponics system of claim 53 wherein said at least one control unit, at least one biometric camera unit and at least one thermal camera unit provide identification, monitoring, tracking and record keeping of at least one plant species, animal species and microorganism.

55. The aquaponics system of claim 53 wherein said at least one control unit provides automated, customized and individualized feeds, nutrients, and supplements to said at least one plant species, animal species and microorganism and reduce or eliminate unwanted insects and vermin in said aquaponics system.

56. The aquaponics system of claim 46 wherein said at least one control unit, at least one biometric camera unit and at least one thermal camera unit provide identification, monitoring, tracking, and record keeping of individuals within said aquaponics system for security purposes.

57. The aquaponics system of claim 46 wherein said at least one control unit communicates with and controls at least one robotic apparatus.

58. The aquaponics system of claim 57 wherein said at least one robotic apparatus provides said at least one control unit with automation, harvesting and processing capabilities to increase the efficiency of said aquaponics system.

59. The aquaponics system of claim 57 wherein said at least one robotic apparatus provides:
a. Transfer of seeds to said at least one germination unit;
b. Transfer of seeds to said at least one grow out unit;
c. Unmanned harvesting and pre-processing of said harvest; and
d. Transfer of said pre-processed harvest to a packaging and storage unit.

60. The aquaponics system of claim 46 wherein said system is a partially closed circuit.

61. The aquaponics system of claim 46 wherein said system is a closed circuit.

62. The aquaponics system of claim 46 wherein minimal to no direct contact is provided between aquatic species waste and said at least one plant species.

63. The aquaponics system of claim 46 wherein minimal amounts of plant growing media is utilized.

64. The aquaponics system of claim 46 wherein no plant growth media is utilized.

65. The aquaponics system of claim 46 wherein said digester unit comprises biological species of at least one worm.

66. The aquaponics system of claim 46 wherein said filter unit comprises means for treating water through nitrification; wherein said nitrification comprises at least one nitrification entity capable of nitrifying ammonia.

67. The aquaponics system of claim 66 wherein said nitrification means comprises a tank for housing said nitrification entity; said nitrification tank being separated from a plant growing apparatus.

68. The aquaponics system of claim 67 wherein said nitrification entity is selected from the group consisting of:
a. Chemicals;
b. Zeolite filters;
c. Nitrifying microorganisms; and
d. Combinations thereof.

69. The aquaponics system of claim 66 wherein said nitrification tank comprises one or more of a baffle, air jets, or water jets and provides reversible unidirectional flow through said nitrification tank.

70. The aquaponics system of claim 67 wherein said plant growing apparatus is a stacked apparatus comprising at least one of a multiple level A frame structure and a ladder style structure.

71. The aquaponics system of claim 46 wherein said system further comprises an insect larvae production module.

72. The aquaponics system of claim 71 wherein said insect larvae production module comprises a reversibly sealable container for housing organic waste and an insect larvae outlet pipe.

73. The aquaponics system of claim 71 wherein said automation provided by said at least one control unit provides improved genetics of aquatic species, plant species, insects, and worms having improved disease resistance, reproduction rates and growth rates within said aquaponics system.

74. The aquaponics system of claim 46 wherein said at least one bioreactor and said at least one microorganism reactor providing cultivation of microalgae and aquatic organisms utilizing nutrient enriched water, wherein said reactors comprise separate enclosures for nutrient enrichment and cultivation of organisms, said bioreactor comprising:
a. At least one first enclosure wherein nutrient enrichment of a water source is effected; and
b. at least one third enclosure containing at least one organism and connected to at least one of said first enclosure.

75. The aquaponics system of claim 74 wherein said at least one first enclosure is connected with said at least one third enclosure through at least one second enclosure, wherein said at least one second enclosure accepts enriched water from said at least one first enclosure to age said enriched water before transferring aged enriched water to said at least one third enclosure.

76. The aquaponics system of claim 74 wherein said at least one first enclosure and said at least one third enclosure are connected through an external water mass, wherein said external water mass is at least one of fresh water and salt water.

77. The aquaponics system of claim 74 wherein said at least one first and third enclosures utilized stirred filters to reduce the size of formed aggregates within said at least one first and third enclosures.

78. The aquaponics system of claim 46 wherein said system further comprises at least one energy re-capture method.

79. The aquaponics system of claim 78 wherein said system provides energy storage through thermal energy capture utilizing heat exchangers, absorption cooling, and a Stirling engine.

80. The aquaponics system of claim 79 wherein said energy storage includes storage of at least one high temperature, medium temperature, low temperate, and cold temperature storage.

81. The aquaponics system of claim 80 wherein said energy-re-capture utilizes at least one of wind energy, solar energy, digester biogas burning energy, and compost generated energy.

82. The aquaponics system of claim 46 wherein at least one of shad and freshwater sardine is grown to be utilized as fish meal for said at least one aquatic animal species.

83. An apparatus for metabolism manipulation of species utilizing at least one light source spectrum output comprising:
a. At least one illumination array;
b. At least one remotely programmable microcontroller, wherein said microcontroller comprises a first processor and a first machine readable medium containing instructions for controlling said at least one illumination array;
c. At least one power source connected to said at least one illumination array and at least one remotely programmable microcontroller; and
d. An interface through said at least one remotely programmable microcontroller Providing for automatic and manual control of said apparatus.

84. The apparatus of claim 83 wherein said species are at least one of a single-cell animal and a multi-cell animal.

85. The apparatus of claim 83 wherein said at least one illumination array comprises at least one of:
a. A first plurality of light sources having a first light spectrum emission;
b. A second plurality of light sources having a second light spectrum emission;
c. A third plurality of light sources having a third light spectrum emission; and
d. Wherein said microcontroller controls said first, second, and third plurality of light sources.

86. The apparatus of claim 85 wherein said first, second, and third light spectrum emission are individually compatible with the photosynthetic growth characteristics of at least one plant species.

87. The apparatus of claim 83 wherein said microcontroller provides commands for each light source within each of said first, second, and third plurality of light sources.

88. The apparatus of claim 83 wherein said microcontroller provides remote access to said first, second, and third plurality of light sources through a computing device comprising a second processor and a second machine readable medium containing instructions for communication with said microcontroller locally or through at least one network.

89. A method for plant metabolism manipulation using spectral output comprising:
a. Determining the photosynthetic properties of a targeted plant species;
b. Fabrication of an illumination array of light sources comprising combinations of desired pluralities of light emissions that are compatible with photosynthetic growth properties of at least one plant species;
c. Placing the targeted at least one plant species in a desirable proximity to said illumination array of light sources; and
d. Operatively connecting a programmable microprocessor to the illumination array of light sources wherein said programmable microprocessor transmits commands to a desired plurality of illumination array light sources to output specific light emissions at least one of a desired time, period, and illumination intensity.

90. The method of claim 89 further including the step of simulating a predawn glow using said illumination array of light sources.

91. The method of claim 89 further including the step of simulating an after sunset glow using said illumination array of light sources.

92. The method of claim 89 further including the step of flashing specific pluralities of illumination array light sources for at least one of a flash interval, desired time, period, and illumination intensity.
